(19) United States
(12) Patent Application Publication
(10) Pub. No.: US 2003/0103939 A1
(43) Pub. Date: Jun. 5, 2003

(54) Pseudotyped adeno-associated viruses and uses thereof

(76) Inventors: John F. Engelhardt, Iowa City, IA (US); Ziying Yan, Iowa City, IA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/194,421

(22) Filed: Jul. 12, 2002

Related U.S. Application Data
(60) Provisional application No. 60/305,204, filed on Jul. 13, 2001.

(51) Int. Cl. A61K 48/00; C12N 7/00; C12N 15/861

(52) U.S. Cl. 424/93.2; 514/44; 435/456; 435/235.1; 435/320.1

(57) ABSTRACT

Pseudotyped rAAV and methods of using pseudotyped rAAV are provided.

### STATEMENT OF GOVERNMENT RIGHTS

This invention was made at least in part with a grant from the Government of the United States of America (grant numbers HL58340 and P30 DK54759 from the National Institutes of Health). The Government may have certain rights in the invention.

### CROSS-REFERENCE TO RELATED APPLICATIONS

This application claims the benefit of the filing date of U.S. application Serial No. 60/305,204, filed Jul. 13, 2001, under 35 U.S.C. §119(e), the disclosure of which is incorporated by reference herein in its entirety.

### BACKGROUND OF THE INVENTION

AAV is currently considered an ideal vehicle for human gene therapy, as it is a small, defective, nonpathogenic, single-stranded DNA virus with the ability to infect non-dividing cells and to establish long-term, latent infection in vivo in a wide variety of organs without immunogenicity (Flotte et al., 1995). For example, promising results were recently obtained from clinical trials with type-2 recombinant AAV (rAAV-2) based gene therapy for hemophilia B (Kay et al., 2000). Moreover, among the non-viral and viral vectors used in muscle gene transfer, rAAV-2 vectors are especially attractive because they can support persistent transgene expression in muscle. Muscle based gene therapy protocols have been widely investigated for inherited muscle diseases such as muscular dystrophies as well as a platform to produce secreted therapeutic proteins. However, further improvements in viral titer may be needed to completely correct functional defects in patients (Ray et al., 2000).

Various strategies have been under development to enhance the potency of rAAV-2 vectors for in vivo use. Hagstrom et al. (2000) have demonstrated higher levels of factor IX production from rAAV-2 vectors by modifying the transgene expression cassette (Hagstrom et al., 2000). In addition, Duan et al. (2000a) observed greater than 200-fold enhancement in rAAV mediated transgene expression in muscle when a second super-enhancer rAAV vector was co-administered. Further, a wide panel of small chemical compounds has been examined to supplement the viral genome directed approaches mentioned above to identify additional means of improving rAAV-2 mediated gene transfer. For instance, dephosphorylation of the single stranded D sequence binding protein has been correlated with the activation of rAAV-2 transduction and, in this context, a series of tyrosine kinase inhibitors has been developed to increase rAAV-2 transduction by enhancing gene conversion (Qing et al., 1998).

Additionally, in an effort to overcome barriers to intracellular trafficking of rAAV-2, a dramatic increase in rAAV-2 transduction was observed in polarized airway cells when proteasome inhibitors were co-administered with the virus (Duan et al., 2000b). Modulation of the ubiquitin-proteosome system may have resulted in significant enhancement of rAAV-2-mediated transgene expression and a concurrent augmentation in nuclear trafficking of virus. Thus, ubiquitination of the AAV-2 capsid proteins might play a role as a barrier to rAAV-2 transduction by rerouting intracellular trafficking to a non-expressible compartment or by promoting viral degradation of incoming virions. Evidence that intracellular trafficking in fibroblasts may be a barrier to AAV-2 transduction has also been described (Hansen et al., 2000; and Hansen et al., 2001).

Further, circularization and/or concatamerization of AAV-2 genomes can overcome the inherent 4.7 kb packaging limitation of rAAV (Duan et al, 1998; Duan et al., 2000a; Nakai et al., 2000; Sun et al., 2000; and Yan et al., 2000). These approaches allow the delivery of large transgenes or a transgene and regulatory element(s) using heterodimerization and trans-splicing of independent AAV-2 vectors.

Recently, the preparation of recombinant viral stocks from additional AAV serotypes was made possible via the cloning of those serotypes (Bantel-Schaal et al., 1999; Chlorini et al., 1999; Chlorini et al., 1997; Muramatsu et al., 1996; Rutledge et al., 1998; and Xiao et al., 1998). Cloning and sequencing of six primate isolates of AAV serotypes indicated that they share similar genomic organization. AAV DNA replication, provirus integration and packaging of progeny AAV DNA into virus particles require a minimal sequence having two large, open reading frames flanked by an inverted terminal repeat (ITR) at each end. The left open reading frame (ORF) encodes 4 non-structural Rep proteins. These proteins are not only the regulators of AAV transcription, but are also involved in AAV replication, virus assembly, and even play a role in site-specific integration of the viral genome into the host chromosome during latent infection. The sequence of the Rep ORFs of AAV-2, AAV-3, AAV-4 and AAV-6 are approximately 85% identical, but AAV-5 has only 54.5% homology with the other AAV serotypes.

The right half of the AAV genome encodes three viral capsid proteins referred to as VP1, VP2 and VP3, and is less conserved than the Rep ORF. Although AAV-2, AAV-3 and AAV-6 share about 80% homology in the amino acid sequences of the capsid proteins, alignment of the capsid ORFs of all the six serotypes results in a reduction of the overall amino acid identity to less than 45% (Bantel-Schaal et al., 1999). The most divergent regions appear to occur at the exterior surface of the mature virion (Bantel-Schaal et al., 1999; and Chlorini et al., 1999). This diversity in the capsid protein sequences is the basis for differences in the serological characteristics and altered tissue tropism among the six AAV serotypes.

In particular, sequence comparisons indicate that the AAV-5 capsid proteins are significantly different from those of the other serotypes. For example, detailed sequence comparisons of the AAV-2 and AAV-5 capsids indicate less than 45% homology, with the most divergent regions on the exterior surface of the virion. AAV-5 likely utilizes a different receptor and/or co-receptor for entering cells. Indeed, distinct transduction profiles between AAV-2 and AAV-5 have been demonstrated in several different cell types, including polarized airway epithelia, muscle and neuronal cells in vivo (Davidson et al., 2000; Zabner et al., 2000; and Hildinger et al., 2001). Moreover, a recent study in NOD/SCID mice has also suggested that AAV-5 might be a better vector for muscle than AAV-2 (Chao et al., 2000). However, none of these studies identified the intracellular step(s) in viral transduction which accounted for the differences.

Thus, what is needed is a method to increase the efficacy of rAAV-mediated gene delivery.

### SUMMARY OF THE INVENTION

The invention provides a method to alter, e.g., enhance, transduction of a eukaryotic cell by pseudotyped recombinant AAV (rAAV) and a method to identify agents that alter transduction by pseudotyped rAAV. A pseudotyped rAAV is an infectious virus comprising any combination of an AAV capsid protein and a rAAV genome. Pseudotyped rAAV are useful to alter the tissue or cell specificity of rAAV, and may be employed alone or in conjunction with non-pseudotyped rAAV to transfer one or more genes to a cell, e.g., a mammalian cell. For example, pseudotyped rAAV may be employed subsequent to administration with non-pseudotyped rAAV in a mammal which has developed an immune response to the non-pseudotyped rAAV. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 ITRs, each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric, i.e., an ITR which has ITR sequences from more than one AAV serotype. In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

Thus, the invention provides a method to identify an agent that alters pseudotyped rAAV transduction of a eukaryotic cell, e.g., a mammalian cell such as a mammalian lung, epithelial, e.g., nasal epithelial, neural, muscle or liver cell, or a population of eukaryotic cells. The method comprises contacting the cell or population of cells with one or more agents and the pseudotyped virus. Then it is determined whether virus transduction is altered, e.g., by detecting expression of a marker gene, selectable gene or a therapeutic gene product. Preferred cells include those of mammals, birds, fish, and reptiles, especially domesticated mammals and birds such as humans, non-human primates, cattle, sheep, pigs, horses, dogs, cats, mice, rats, rabbits, chickens, and turkeys. Preferred agents are those which enhance virus transduction, e.g., by enhancing viral endocytosis, decreasing viral nucleic acid or protein degradation in endosomes or in proteosomes, enhancing endosomal processing and/or enhancing viral transport to the nucleus. Thus, agents which enhance virus transduction are particularly useful in gene therapy which employs rAAV to introduce and/or express a therapeutic peptide or polypeptide. Further, the cells to be transduced may be contacted with the one or more agents prior to viral infection, concurrently with viral infection, subsequent to viral infection, or any combination thereof.

As described hereinbelow, rAAV-2 genomes were packaged into AAV-5 capsids in the presence of complementing AAV-2 Rep proteins, yielding infectious particles. For a direct comparison of the infection pathways for rAAV vectors of different serotypes, rAAV was also prepared having rAAV-2 genomes packaged into AAV-2 capsids. Then the efficiency of gene delivery to mouse muscle cells for rAAV-2 and rAAV-2cap5 (AAV-2 genomes pseudo-packaged into AAV-5 capsids) was compared. Despite similar levels of transduction by these two vectors in undifferentiated myoblasts, pseudotyped rAAV-2cap5 demonstrated dramatically enhanced transduction in differentiated myocytes in vitro (>500-fold) and in skeletal muscle in vivo (>200-fold) as compared to rAAV-2. Serotype specific differences in transduction efficiency did not directly correlate with viral binding to muscle cells but rather appeared to involve endocytic or intracellular barriers to infection. Furthermore, the pseudotyped virus also demonstrated significantly improved transduction efficiency in a mouse model of Duchenne's muscular dystrophy.

As also described hereinbelow, the transduction efficiency of a recombinant AAV-2 construct with an RSV LTR promoter driving a luciferase reporter that was packaged into both AAV-2 and AAV-5 capsid particles was compared in a number of cell lines and in lung in vivo. Co-administration of the viruses with proteosome inhibitors in vitro not only increased the transduction efficiency of AAV-2, it also augmented AAV-5 mediated gene transfer although often to a slightly lower extent. Increased transgene expression in the presence of proteasome inhibitor was independent of viral genome degradation since no significant difference of the amount of internalized viral DNA was detected 24 hours after infection. Western blot assays of immunoprecipitated viral proteins from infected HeLa cell lysates and in vitro reconstitution experiments revealed evidence for ubiquitin conjugation of both AAV-2 and AAV-5 capsids. These studies suggest that the previously reported barrier involving the ubiquitin/proteasome pathway for rAAV-2 is also active for rAAV-5 capsid entry pathways. In vivo co-administration of a pseudotyped rAAV and the proteosome inhibitor Z-LLL induced whole lung luciferase expression 17.2- and 2.1-fold at 14 and 42 days post-infection, respectively.

Agents to enhance the transduction of cells, e.g., human cells, by rAAV include endosomal protease or proteosome inhibitors including but not limited to cysteine protease inhibitors such as a peptide cysteine protease inhibitor, e.g., LLnL, or an analog thereof. Therefore, the invention further provides a method in which a eukaryotic cell is contacted with virus and an agent comprising a compound of formula (I): R₁-A-(B)ₙ-C, wherein R₁ is an N-terminal amino acid blocking group; each A and B is independently an amino acid; C is an amino acid wherein the terminal carboxy group has been replaced by a formyl (CHO) group; and n is 0, 1, 2, or 3; or a pharmaceutically acceptable salt thereof. In one embodiment, R₁ is (C₁-C₁₀)alkanoyl. In another embodiment, R₁ is acetyl or benzyloxycarbonyl. In yet another embodiment, R₁ is (C₁-C₁₀)alkanoyl or benzyloxycarbonyl; A and B are each isoleucine; C is nor-leucine or nor-valine, wherein the terminal carboxy group has been replaced by a CHO group; and N is 1. In a further embodiment, C is alanine, arginine, glycine, isoleucine, leucine, valine, nor-leucine or nor-valine, wherein the terminal carboxy group has been replaced by a CHO group, e.g., in one embodiment C is nor-leucine or nor-valine and the terminal carboxy group is replaced by a CHO group. In yet a further embodiment, A and B are each independently alanine, arginine, glycine, isoleucine, leucine, valine, nor-leucine or nor-valine, e.g., in one embodiment A and B are each isoleucine.

Another agent of the invention is a compound of formula (II):

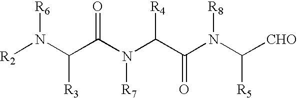

wherein

R₂ is an N-terminal amino acid blocking group;

R₃, R₄, and R₅ are each independently hydrogen, (C₁-C₁₀)alkyl, aryl or aryl(C₁-C₁₀)alkyl; and

R₆, R₇, and R₈ are each independently hydrogen, (C₁-C₁₀)alkyl, aryl or aryl(C₁-C₁₀)alkyl; or a pharmaceutically acceptable salt thereof.

R₂ may be (C₁-C₁₀)alkanoyl, e.g., acetyl or benzyloxycarbonyl; R₃ may be hydrogen or (C₁-C₁₀)alkyl, e.g., 2-methylpropyl. R₅ may be hydrogen or (C₁-C₁₀)alkyl, e.g., butyl or propyl. In one embodiment, R₂ is acetyl or benzyloxycarbonyl; R₃ and R₄ are each 2-methylpropyl; R₅ is butyl or propyl; and R₆, R₇, and R₈ are each independently hydrogen. In one embodiment, R₁ is H, halogen, (C₁-C₁₀)alkyl, (C₁-C₁₀)alkenyl, (C₁-C₁₀)alkynyl, (C₁-C₁₀)alkoxy, (C₁-C₁₀)alkanoyl, (═O), (═S), OH, SR, CN, NO₂, trifluoromethyl or (C₁-C₁₀)alkoxy, wherein any alkyl, alkenyl, alkynyl, alkoxy or alkanoyl may optionally be substituted with one or more halogen, OH, SH, CN, NO₂, trifluoromethyl, NRR or SR, wherein each R is independently H or (C₁-C₁₀)alkyl; R₂ is (═O) or (═S); R₃ is H, (C₁-C₁₀)alkyl, (C₁-C₁₀)alkenyl, (C₁-C₁₀)alkynyl, (C₁-C₁₀)alkoxy or (C₃-C₈)cycloalkyl, wherein any alkyl, alkenyl, alkynyl, alkoxy or cycloalkyl may optionally be substituted with one or more halogen, OH, CN, NO₂, trifluoromethyl, SR, or NRR, wherein each R is independently H or (C₁-C₁₀)alkyl; R₄ is H, (C₁-C₁₀)alkyl, (C₁-C₁₀)alkenyl, (C₁-C₁₀)alkynyl, (C₁-C₁₀)alkoxy or (C₃-C₈)cycloalkyl, wherein any alkyl, alkenyl, alkynyl, alkoxy or cycloalkyl may optionally be substituted with one or more halogen, OH, CN, NO₂, trifluoromethyl, SR, or NRR, wherein each R is independently H or (C₁-C₁₀)alkyl; R₅ is H, halogen, (C₁-C₁₀)alkyl, (C₁-C₁₀)alkenyl, (C₁-C₁₀)alkynyl, (C₁-C₁₀)alkoxy, (C₁-C₁₀)alkanoyl, (═O), (═S), OH, SR, CN, NO₂ or trifluoromethyl, wherein any alkyl, alkenyl, alkynyl, alkoxy or alkanoyl may optionally be substituted with one or more halogen, OH, SH, CN, NO₂, trifluoromethyl, NRR or SR, wherein each R is independently H or (C₁-C₁₀)alkyl; and X is O, S or NR wherein R is H or (C₁-C₁₀)alkyl, or a pharmaceutically acceptable salt thereof.

Other agents useful in the methods of the invention include a compound of formula (III):

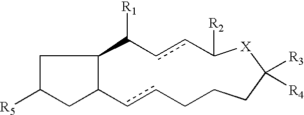

wherein,

R₁ is H, halogen, (C₁-C₁₀)alkyl, (C₁-C₁₀)alkenyl, (C₁-C₁₀)alkynyl, (C₁-C₁₀)alkoxy, (C₁-C₁₀)alkanoyl, (═O), (═S), OH, SR, CN, NO₂, trifluoromethyl or (C₁-C₁₀)alkoxy, wherein any alkyl, alkenyl, alkynyl, alkoxy or alkanoyl may optionally be substituted with one or more halogen, OH, SH, CN, NO₂, trifluoromethyl, NRR or SR, wherein each R is independently H or (C₁-C₁₀)alkyl; R₂ is (═O) or (═S); R₃ is H, (C₁-C₁₀)alkyl, (C₁-C₁₀)alkenyl, (C₁-C₁₀)alkynyl, (C₁-C₁₀)alkoxy or (C₃-C₈)cycloalkyl, wherein any alkyl, alkenyl, alkynyl, alkoxy or cycloalkyl may optionally be substituted with one or more halogen, OH, CN, NO₂, trifluoromethyl, SR, or NRR, wherein each R is independently H or (C₁-C₁₀)alkyl; R₄ is H, (C₁-C₁₀)alkyl, (C₁-C₁₀)alkenyl, (C₁-C₁₀)alkynyl, (C₁-C₁₀)alkoxy or (C₃-C₈)cycloalkyl, wherein any alkyl, alkenyl, alkynyl, alkoxy or cycloalkyl may optionally be substituted with one or more halogen, OH, CN, NO₂, trifluoromethyl, SR, or NRR, wherein each R is independently H or (C₁-C₁₀)alkyl; R₅ is H, halogen, (C₁-C₁₀)alkyl, (C₁-C₁₀)alkenyl, (C₁-C₁₀)alkynyl, (C₁-C₁₀)alkoxy, (C₁-C₁₀)alkanoyl, (═O), (═S), OH, SR, CN, NO₂ or trifluoromethyl, wherein any alkyl, alkenyl, alkynyl, alkoxy or alkanoyl may optionally be substituted with one or more halogen, OH, SH, CN, NO₂, trifluoromethyl, NRR or SR, wherein each R is independently H or (C₁-C₁₀)alkyl; and X is O, S or NR wherein R is H or (C₁-C₁₀)alkyl, or a pharmaceutically acceptable salt thereof.

Preferably, R₁ is OH. It is also preferred that R₂ is (═O); R₃ is H or (C₁-C₁₀)alkyl, and more preferably R₃ is methyl. Other preferred embodiments include R₄ is H or (C₁-C₁₀)alkyl, and more preferably, R₄ is H; R₅ is halogen, CN, NO₂, trifluoromethyl or OH, and more preferably, R₅ is OH. A compound of formula (III) includes X is O or S, preferably O; wherein both ----- are a single bond, wherein one ----- is a double bond, or wherein both ----- are a double bond. In a more preferred embodiment, R₁ is OH, R₂ is (═O), R₃ is methyl, R₄ is H, R₅ is OH, X is O, and both ----- are a double bond.

Yet another agent useful in the methods of the invention is a compound of formula (III):

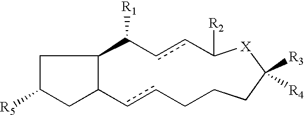

wherein R₁ is halogen, CN, NO₂, trifluoromethyl or OH. Preferably, R₁ is OH. It is also preferred that R₂ is (—O); R₃ is H or (C₁-C₁₀)alkyl, and more preferably R₃ is methyl. Other preferred embodiments include R₄ is H or (C₁-C₁₀)alkyl, and more preferably, R₄ is H; R₅ is halogen, CN, NO₂, trifluoromethyl or OH, and more preferably, R₅ is OH. A compound of formula (III) includes X is O or S, preferably O; wherein both ----- are a single bond, wherein one ----- is a double bond, or wherein both ----- are a double bond. In a more preferred embodiment, R₁ is OH, R₂ is (═O), R₃ is methyl, R₄ is H, R₅ is OH, X is O, and both ----- are a double bond.

Another agent useful in the methods of the invention includes an agent that inhibits the activation of ubiquitin, the transfer of ubiquitin to the ubiquitin carrier protein, ubiquitin ligase, or a combination thereof. Preferred ubiquitin ligase inhibitors include a compound of formula (IV):

R—A—A₁—R₁

wherein R is hydrogen, an amino acid, or a peptide, wherein the N-terminus amino acid can optionally be protected at the amino group with acetyl, acyl, trifluoroacetyl, or benzyloxycarbonyl;

A is an amino acid or a direct bond;

A₁ is an amino acid; and

R₁ is hydroxy or an amino acid, wherein the C-terminus amino acid can optionally be protected at the carboxy group with (C₁-C₆)alkyl, phenyl, benzyl ester or amide (e.g., C(═O)NR₂, wherein each R is independently hydrogen or (C₁-C₆)alkyl);

or a pharmaceutically acceptable salt thereof.

A specific value for R is hydrogen.

A specific value for A is an amino acid. Another specific value for A is Ile, Leu or His. Another specific value for A is Leu or His.

A specific value for A₁ is Ala or Gly. Another specific value for A₁ is Ala.

A specific value for R₁ is hydroxy.

Specifically, the peptide can be a dipeptide (i.e., can comprise 2 amino acids).

Specifically, the peptide can be H-Leu-Ala-OH, H-His-Ala-OH, H-Leu-Gly-OH, H-His-Gly-OH, H-Ile-Ala-OH, or H-Ile-Gly-OH. More specifically, the peptide can be H-Leu-Ala-OH or H-His-Ala-OH.

Further, the activity of agents that inhibit processing, e.g., endosomal processing, of virus may be enhanced by the addition of agents, such as EDTA or EGTA, which may alter molecules in pathways associated with endosomal processing, e.g., agents such as calcium chelators or modulators of intracellular calcium levels. Thus, a combination of agents including inhibitors of endosomal processing and an agent that enhances the activity of the inhibitor(s) may be employed in the methods of the invention.

The invention also provides a method to alter rAAV transduction of a eukaryotic cell or a population of cells. The method comprises contacting the cell or population of cells with one or more rAAV, e.g., a pseudotyped rAAV, and at least one agent in an amount effective to alter virus transduction. The agent may be contacted with the cell concurrently with virus, prior to contacting the cell with virus or after contacting the cell with virus. The agent(s) and/or virus may each be administered once, or in repeated dosing, so as to achieve the desired effect, i.e., to enhance rAAV transduction. Since AAV has been shown to have a broad host range (for pulmonary expression) and persists in muscle, rAAV may be employed to express a gene in any animal, and particularly in mammals, birds, fish, and reptiles, especially domesticated mammals and birds such as cattle, sheep, pigs, horses, dogs, cats, chickens, and turkeys. Both human and veterinary uses are particularly preferred. The gene being expressed can be either a DNA segment encoding a polypeptide, with whatever control elements (e.g., promoters, operators) are desired, or a non-coding DNA segment, the transcription of which produces all or part of some RNA-containing molecule (such as a transcription control element, +RNA, or anti-sense molecule). In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

In particular, the pseudotyped rAAV of the invention and optionally one or more agents of the invention may be employed in methods to alter, e.g., increase, transduction efficiency and/or transgene expression, methods to detect or determine transgene expression efficiency, methods to screen for promoter strength and/or RNA stability, as well as in therapeutic or prophylactic therapies including therapies for blood disorders (e.g., sickle cell anemia, thalassemias, hemophilias, and Fanconi anemias), neurological disorders, such as Alzheimer's disease and Parkinson's disease, and muscle disorders involving skeletal, cardiac or smooth muscle, as well as diseases of the lung, e.g., cystic fibrosis and asthma. In particular, pseudotyped rAAV may be employed to deliver therapeutic genes including but not limited to the 13-globin gene, the gamma-globin gene, the Factor VIII gene, the Factor 1×gene, the cystic fibrosis transmembrane conductance receptor (CFTR) gene, the erythropoietin (epo) gene, the Fanconi anemia complementation group, a gene encoding a ribozyme, an antisense gene, a low density lipoprotein (LDL) gene, a tyrosine hydroxylase gene (Parkinson's disease), a glucocerebrosidase gene (Gaucher's disease), an arylsulfatase A gene (metachromatic leukodystrophies), a dystrophin gene, a dysferlin gene, an ATP binding cassette transporter gene, or genes encoding other polypeptides or proteins. Also within the scope of the invention is the inclusion of more than one gene or open reading frame in a vector of the invention, i.e., a plurality of genes may be present in an individual vector.

Further, co-infection with two or more different rAAV may, through intermolecular recombination, yield a concatamer having one or more copies of any particular rAAV. The implications of intermolecular recombination of rAAV genomes to form a single molecule, e.g., an episome, which may be a concatamer comprising at least two different rAAV genomes, is particularly relevant for gene therapy with rAAV as large regulatory elements and genes beyond the packaging capacity of rAAV can be brought together by co-infecting cells or tissue of an organism with two independent rAAV vectors. For example, enhancers and/or promoters may be introduced into one vector while DNA comprising an open reading frame, e.g., a gene of interest, with or without a minimal promoter, is introduced into a second vector. Thus, after co-infection with the two vectors, the transgene cassette size is increased beyond that for a single AAV vector alone and the DNA comprising the opening reading frame is linked to the enhancer and/or promoter. In another embodiment of the invention, vectors encoding two independent regions of a gene are brought together to form an intact splicing unit. In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid. For example, in an embodiment where rAAVs are employed to transduce muscle, the capsid of the rAAV is encoded by the cap gene of serotype AAV-1 or AAV-5 and rep protein and ITRs of the rAAV are from serotype AAV-2 or AAV-1, respectively.

Thus, the present invention is useful to overcome the current size limitation for transgenes within rAAV vectors, and allows for the incorporation of a larger transcriptional regulatory region, e.g., a stronger heterologous promoter or an endogenous CFTR promoter, e.g., the CFTR endogenous promoter, or one or more enhancer sequences.

In a further embodiment of the invention, a vector comprising an origin of replication and a DNA encoding a protein that binds to the origin and promotes replication and/or maintenance of DNA which is linked to the origin, and another vector comprising a gene of interest, are brought together after co-infection to form an episome, preferably an autonomously replicating episome, comprising the gene.

In one embodiment, the origin of replication and DNA encoding the protein are from EBV, e.g., OriP and EBNA-1. In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

Therefore, a plurality of DNA segments, each in an individual rAAV vector, may be delivered to a cell, so as to result in a single DNA molecule having a plurality of the DNA segments from more than one rAAV. In one embodiment of the invention, one rAAV may comprise a first recombinant DNA molecule comprising linked: i ) a first DNA segment comprising a 5′-ITR of AAV; ii) a second DNA segment which does not comprise AAV sequences; and iii) a third DNA segment comprising a 3′-ITR of AAV. A second recombinant AAV comprises a second recombinant DNA molecule comprising linked: i) a first DNA segment comprising a 5′-ITR of AAV; ii) a second DNA segment which does not comprise AAV sequences and which second DNA segment is different than the second DNA segment of the first recombinant DNA molecule; and iii) a third DNA segment comprising a 3′-ITR of AAV. At least one of the rAAV is a pseudotyped rAAV.

Thus, in one embodiment of the invention, one rAAV vector comprises a first DNA segment comprising a 5′ ITR linked to a second DNA segment comprising a promoter operably linked to the 5′ end of an open reading frame (but not the entire open reading frame) and a 5′ splice site linked to a third DNA segment comprising a 3′ ITR. The second rAAV vector comprises a first DNA segment comprising a 5′ ITR linked to a second DNA segment comprising a 3′ splice site and the 3′ end (the remainder) of the open reading frame, i.e., the second DNA segment of the second vector together with the second DNA segment of the first vector encodes a functional peptide or polypeptide, linked to a third DNA segment comprising a 3′ ITR. A “functional” peptide or polypeptide is one which has substantially the same activity as a reference peptide or polypeptide, for example, a wild-type (full-length) polypeptide. Preferably, the second DNA segments together comprise DNA encoding, for example, CFTR, factor VIII, dystrophin, or erythropoietin. The second DNA segments may be obtained or derived from cDNA, genomic DNA or a combination thereof. For example, the second DNA segment of the first vector may comprise one or more, but not all of the exons of a gene comprising more than one exon and the second DNA segment of the second vector may comprise at least one exon of the gene that is not present in the first vector. The second DNA segment of the first vector may comprise the endogenous promoter of the respective gene, e.g., the epo promoter. In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

In another embodiment, one rAAV vector comprises a first DNA segment comprising a 5′ ITR linked to a second DNA segment comprising a promoter and/or enhancer linked to a third DNA segment comprising a 3′ ITR. A second rAAV vector comprises a first DNA segment comprising a 5′ ITR linked to a second DNA segment comprising at least a portion of an open reading frame optionally linked to a promoter (a different promoter than in the first vector or a second copy of the promoter in the first vector) linked to a third DNA segment comprising a 3′ ITR. For example, the second DNA segment of the first recombinant DNA molecule comprises at least one heterologous enhancer and/or at least one heterologous promoter, i.e., the enhancer and/or promoter sequences are not derived from AAV sequences. Preferably, the second DNA segment of the second recombinant DNA molecule comprises a portion of an open reading frame which encodes a functional protein. Thus, co-infection of a cell with at least one pseudotyped rAAV, e.g., a transgene containing vector, and a second vector comprising at least one, preferably at least two or more, enhancer sequences, can result in an enhancement of transgene expression from a minimal promoter. Furthermore, an enhancement can also be achieved by cis-activation of ITRs in transgene-containing vectors without a promoter. Thus, large regulatory elements including tissue-specific enhancers can be introduced into cells by a separate rAAV vector to regulate the expression of a second transgene-containing AAV vector in cis following intracellular concatamerization. In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

In yet a further embodiment of the invention, the second DNA segment of the first recombinant DNA molecule comprises an origin of replication functional in a host cell, e.g., a viral origin of replication such as OriP. Preferably, the origin is functional in a human cell. Also preferably, the second DNA segment of the first recombinant DNA molecule further comprises DNA encoding a protein that binds to the origin of replication, e.g., EBNA-1. The second DNA segment in the second recombinant DNA molecule comprises at least a portion of an open reading frame, and preferably a promoter operably linked to the open reading frame.

In yet another embodiment of the invention, the second DNA segment of the first recombinant DNA molecule comprises a cis-acting integration sequence(s) for a recombinase and also encodes a recombinase or integrase that is specific for the integration sequence(s), e.g., Cre/lox system of bacteriophage P1 (U.S. Pat. No. 5,658,772), the FLP/FRT system of yeast, the Gin recombinase of phage Mu, the Pin recombinase of E. coli, the R/RS system of the pSR1 plasmid, a retrotransposase or the integrase from a lentivirus or retrovirus. The second DNA segment in the second recombinant DNA molecule comprises at least a portion of an open reading frame, and preferably a promoter operably linked to the open reading frame. The formation of a concatamer comprising the first and the second recombinant DNA molecules, and the expression of the recombinase or integrase, will enhance the integration of the concatamer, or a portion thereof, into the host genome. Also, rAAV vectors comprising cis-acting integration sequences and the corresponding recombinase or integrase are useful to drive directional recombination, which, as dicussed above, may be particularly useful when employing two or more rAAV vectors. In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

Thus, the vectors of the invention are useful in a method of delivering and/or expressing one or more genes in a host cell, to prepare host cells having the vector(s), and in the preparation of a composition comprising rAAV(s). A host cell may be contacted with each rAAV individually, e.g., sequentially, with or without an agent of the invention. To deliver the gene(s) to the host cell, a recombinant adenovirus helper virus may be employed.

Thus, the invention also provides a method to express a polypeptide in a host cell. The host cell is preferably a mammalian host cell, e.g., a murine, canine, feral or human cell, and may be a lung, neuron or muscle cell. The method comprises contacting the host cell with at least two rAAV vectors, at least one of which is a pseudotyped rAAV. The host cell is preferably contacted with the vectors concurrently, although it is envisioned that the host cell may be contacted with each vector at a different time relative to the contact with the other vector(s). One or more agents of the invention may also be employed in the method and may be contacted with the cell prior to, concurrent with, or subsequent to contact of the cell with the vector(s). In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

Also provided is a method to detect expression of a transgene in a cell. The method comprises contacting a host cell with a pseudotyped rAAV of the invention which comprises a transgene comprising a non-AAV promoter linked to an open reading frame, e.g., a marker gene or an open reading frame having one or more genetic modifications relative to a corresponding wild-type open reading frame.

The expression of the transgene is then detected or determined, e.g., relative to a host cell contacted with a rAAV comprising a transgene linked to a different promoter or a transgene with the same promoter but linked to a wild-type open reading frame. Optionally, the cell may be contacted with one or more agents of the invention.

The invention also provides a cell contacted with a rAAV and an agent which alters virus transduction. In one embodiment, the cell is contacted with rAAV comprising AAV-5 capsid and an agent which alters virus transduction. In another embodiment, the cell is contacted with rAAV which is pseudotyped and an agent which alters virus transduction. In one embodiment, the capsid of the rAAV is encoded by the cap gene of serotype AAV-5 and rep protein and ITRs of the rAAV are from serotype AAV-2. In other embodiments, the capsid of the rAAV is encoded by the cap gene of one of serotypes 1-6 of AAV and rep protein and ITRs of the rAAV are from a serotype of AAV that is heterologous to the serotype of the capsid.

### BRIEF DESCRIPTION OF THE FIGURES

FIG. 1. Production of rAAV-2 and rAAV-2cap5 virus. The principles underlying the pseudotyping of rAAV-2 genomes into AAV-5 particles is schematically illustrated in Panel A. In the presence of AAV-2 Rep proteins and helper adenovirus, sequences flanked by the rAAV-2 ITRs are excised from the proviral plasmid (pcisAVV-2) and replicated. Depending on the serotype of capsid proteins provided by a second trans plasmid, the rAAV-2 genome can be packaged in either native AAV-2 or AAV-5 pseudotyped particles. Panel B shows the various helper plasmids that were tested for packaging rAAV-2 DNA into AAV-5 particles. AAV-2 Rep proteins are necessary for pseudo-packaging rAAV-2 genome into AAV-5 particles, and were provided by the helper plasmid, pAV2-Rep. This plasmid was derived from pAAV-2/Ad, the routine helper plasmid for rAAV-2 production, by deleting the AAV-2 capsid coding region. pAV5-Trans was generated by replacing the AAV-2 genome with the full length AAV-5 Rep and Cap coding sequence. It can be used as the helper for generation authentic rAAV-5 vectors or for pseudotyping AAV-2 in an AAV-5 capsid. The AAV-5 capsid expression plasmid, p40Av5Cap(1614), encodes the original p40 promoter for Cap gene transcription. pCMVAv5Cap(1924) is similar, except that the hCMV promoter/enhancer replaces the p40 promoter. pCMVAv5Cap(2196) is derived from pCMVAv5Cap(1924) with the splicing signal deleted so that the CMV promoter is immediately upstream of the VP 1 start code. The effect of the different AAV-5 helper plasmids on virus production is given in Panel C, virus yields of the rAAV-2 and rAAV-2cap5 virus are the mean (+/−SEM) of three independent preparations.

FIG. 2. Myoblast differentiation increases transduction with rAAV-2cap5 but not rAAV-2 virus. Infection of undifferentiated (Panels A, B, C and D) and differentiated (Panels E, F, G and H)C2C12 cells was evaluated for EGFP transgene expression following infection with 3000 DNA particles/cell of either rAAV-2 (Panels A, B, E, and F) or rAAV-2cap5 virus (Panels C, D, G and H) for 24 hours. EGFP expression was evaluated 72 hours after infection by fluorescent microscopy. Nomarski and fluorescent photomicrographs are presented to the left and right of each panel respectively. Quantitative analysis of the percentage of EGFP expressing cells is given in Panel I. Values represent the mean (+/−SEM) for greater than 15 quantitated 10×fields from three independent experiments.

FIG. 3. Quantitative analysis of RSV-luciferase expression from rAAV-2 and rAAV-2capS virus in differentiated and undifferentiated C2C 12 cells. Undifferentiated and differentiated C2C 12 cells were infected with either rAAV-2 or rAAV-2cap5 virus for 24 hours at an moi of 3000 DNA particles/cell (Panel A). Mock-infected cells were used as a negative control for background enzyme activity. The luciferase activity was determined at 24, 48 and 72 hours after infection. The ratio of relative luciferase expression (rAAV-2cap5/rAAV-2) for the two vector types is shown in Panel B. Values in Panels A and B represent the mean (+/−SEM) for three independent data points.

FIG. 4. Examination of viral binding in C2C12 cells. Viral binding was assessed following 4° C. infection of C2C12 cells by Southern blot analysis of viral DNA (Panel A). C2C12 cells were pre-cooled at 4° C. for 10 minutes. After washing with serum-free DMEM, rAAV-2 (lanes 5, 6, 11 and 12) or rAAV-2cap5 (lanes 2, 3, 8 and 9) viruses (carrying the AAV-2 CMV-EGFP cassette) were applied to the cells at an moi of 2000 particles/cell for 60 minutes at 4° C. Mock infected cells were included as negative controls (lanes 1, 4, 7 and 10). At the end of incubation, cells were either washed with PBS alone (lanes 1, 3, 4, 6, 7, 9, 10, and 12) or treated with 0.5% trypsin (lanes 2, 5, 8 and 11) before washing. Hirt DNA was then prepared and analyzed by Southern blot with a transgene (EGFP) specific ³²P-labeled probe. Viral binding from three independent experiments was quantified by densitometry in Panel B (mean+/−SEM). Lane numbers in panel B correspond to the labeling in panel A. Mock: mock-infected cells. Pseudo: rAAV-2cap5 virus. AAV-2: native rAAV-2 virus.

FIG. 5. Proteasome inhibitors differentially affect rAAV-2 and rAAV-2cap5 transduction in differentiated C2C12 cells. To analyze the effect of proteasome inhibitors on the intracellular processing of different AAV serotype, fully differentiated C2C 12 cells were infected with either rAAV-2 or rAAV-2cap5 luciferase vectors at an moi of 600 DNA particles/cell for 4 hours. Tripeptide proteasome inhibitors (40 μM LLnL or 4 μM Z-LLL) were also added to the media during the infection period. Luciferase expression was quantified at 24 hours post-infection. The data represents the mean (+/−SEM) for three independent samples for each experimental condition.

FIG. 6. The AAV-5 receptor is upregulated following differentiation of C2C12 cells. To correlate increased transduction of rAAV-2cap5 in differentiated C2C12 cells with AAV-5 receptors, cell surface alpha-2,3-linked sialic acid expression was determined using a MAL II lectin binding assay. MAL II lectin binding was visualized in undifferentiated (Panels A and B) and differentiated (Panels C and D) C2C12 cells using indirect avidin-FITC fluorescent microscopy (Panels B and D). Panels A and C represent Nomarski photomicrographs of panels B and D, respectively. Increased AAV-5 receptor expression in fully differentiated cells is clearly demonstrated in panel D.

FIG. 7. Factors affecting rAAV binding in C2C12 cells. The effects of heparin competition or sialidase (NA III) treatment on rAAV-2 and rAAV-2cap5 virus infection in C2C12 cells were evaluated (Panel A). rAAV-2 or rAAV-2cap5 infections (moi of 1000 DNA particles/cell) of undifferentiated (lanes 1-6) or differentiated (lanes 7-12) C2C12 cells were evaluated following no treatment (lanes 3, 6, 9, and 12), sialidase treatment (lanes 1, 4, 7, and 10), or heparin (20 μg/ml final concentration) competition (lanes 2, 5, 8, and 11). Hirt DNA was harvested after incubation at 4° C. for 60 minutes and evaluated by Southern blotting against a ³²P-labeled EGFP probe. Panel B depicts results from densitometric quantification of DNA signals from three independent experiments. Values are represented as the percent of inhibition (mean+/−SEM, N=3) in binding following sialidase treatment or heparin competition as compared to untreated controls. Pseudo: rAAV-2cap5 virus.

FIG. 8. Kinetic analysis of rAAV viral genome persistence in differentiated C2C12 cells. To better understand rAAV transduction in myotubes, differentiated C2C12 cells were infected with either rAAV-2cap5 (lanes 1, 2, and 3) or rAAV-2 (lanes 4, 5, and 6) at an moi of 1000 DNA particles/cell. Hirt DNA was harvested at 90 minutes (lanes 1 and 4), 24 hours (lanes 2 and 4) and 48 hours (lanes 3 and 6) post-infection. The left panel depicts a Southern blot hybridized with a ³²P labeled EGFP probe. The right panel depicts the corresponding ethidium bromide stained gel. The lane labels in both panel are identical with the exception of the DNA ladder. Pseudo: rAAV-2cap5 virus.

FIG. 9. A kinetic comparison of EGFP expression in normal and dystrophic muscles. The anterior tibialis muscles of 6-month-old normal or mdx mice were infected with 2×10¹⁰ particles of the indicated viruses. EGFP expression was determined at different time points by fluorescent microscopy. Panels A to H show photographs of whole mount tissue from the freshly excised muscles 1 week and 1 month after infection. Representative photographs from triplicate experiments are shown. Photomicrographs A, B, E and F were taken at an 8 second exposure time. Photomicrographs C, D, G and H were at a 1 second exposure time. EGFP expression 6 months after infection of mdx tibialis muscles 10 was evaluated in paraformaldehyde-fixed, cryopreserved tissue sections (15 μm) following Evan's blue perfusion to demarcate damaged myofibers (I-N).

Photomicrographs in I-K (rAAV-2 infection) were taken from the right leg and in L-N (rAAV-2cap5 infection) were taken from the left leg of the same mouse.

Photomicrographs in panels I and L were 15 seconds exposures and in J, K, M, and N were 2 second exposures. FITC photomicrographs are represented in panels I, J, L and M. Panels J and M (FITC channel) are identical to fields shown in panels K and N (Evans blue, Rhodomin Channel), respectively.

FIG. 10. Quantitative examination of luciferase activity following rAAV-2cap5 or rAAV-2 infection of tibialis muscles. rAAV luciferase expression vectors were used to evaluate transgene expression in normal and mdx anterior tibialis muscles at 1 week and 1 month post-infection with 2×10¹⁰ particles of rAAV-2 (AV2) or rAAV-2cap5 (AV2/5). The data represent the mean (+/−SEM) relative luciferase activity per mg tissue for 3 independent muscle samples from each experimental group.

FIG. 11. Evaluations of the native and pseudotyped rAAV-2 vectors.

Both the native rAAV-2 virus and the AAV-5 pseudotyped virus (rAAV-2cap5) contained the same luciferase reporter derived from the proviral plasmid pcisAV2RSVluc. The titers of both viral stocks used for the study were adjusted to equivalent physical particles/ml. Titration of these two recombinant viral stocks by slot blotting against plasmid DNA standards is shown in Panel A. Panel B illustrates differences in the transduction efficiencies following infection with either native rAAV-2 and pseudotyped rAAV-2cap5 virus in a series of cell types (HeLa cells, primary fetal fibroblasts, IB3 cells, 293 cells, and undifferentiated or differentiated C2C12 muscle cells. Experiments were performed by infecting cells with 5×10⁸ total particles in twelve well plates. The luciferase activity was determined at 24 hours post-infection. Data represents the mean (+/−SEM) for four independent experiments. Panel C compares the time course of transgene expression and viral genome persistence in HeLa cells following infection with rAAV-2 or rAAV-2cap5. 1×10⁹ particles of rAAV-2 (open triangle) or rAAV-2cap5 (filled circle) were used for infection of 6 well plates and luciferase activity was assayed at 24 hours, 48 hours and 72 hours post-infection. Data represented mean (+/−SEM) for three independent experiments (left panel). Low molecular weight Hirt DNA was also harvested from infected HeLa cells at 24 hours (lanes 1 and 4), 48 hours (lanes 2 and 5) and 72 hours (lanes 3 and 6) time points and separated on a 1% agarose gel for Southern blotting with a P³²-labeled luciferase probe (right panel). Lanes 1 to 3 are from rAAV-2 infected cells while lanes 4-6 are from rAAV-2cap5 infected cells.

FIG. 12. Effect of proteosome inhibitors on rAAV-2 and rAAV-2cap5 transduction. HeLa cells were infected with rAAV-2 or rAAV-2cap5 luciferase expressing viruses at an MOI of 250 particles/cell in the presence of different dosages of the proteosome inhibitors LLnL or ZLL (Panel A). HeLa cells were infected with different doses of rAAV-2 or rAAV-2cap5 in the presence of 40 μM LLnL (Panel B). In all panels, luciferase activity was measured at 24 hours post-infection and the data represented the mean (+/−SEM) for four independent experiments

FIG. 13. Ubiquitination of AAV-2 and AAV-5 capsid proteins. Panel A demonstrates Western blot analysis for ubiquitinated AAV-2 and AAV-5 capsid proteins in HeLa cells. HeLa cells were infected with rAAV-2 or rAAV-2cap5 luciferase expressing virus with or without the presence of 40 μM LLnL. Four hours after infection, cells were trypsinized, washed twice with PBS, then lysed in 1 ml RIPA buffer. Virus from HeLa cell lysates was immunoprecipitated with B1 antibody and subject to Western blotting against anti-ubiquitin monoclonal antibody. Lane 1: rAAV-2 infection without LLnL; lane 2: rAAV-2 infection with LLnL; Lane 3: mock-infected cells without LLnL, lane 4: mock-infected cells with LLnL; lane 5: rAAV-2cap5 infection without LLnL; lane 6: rAAV-2cap5 infection with LLnL. Panel B presents Southern blot analysis of low molecular weight Hirt DNA from HeLa cells infected with rAAV-2 (lanes 1 and 2) or rAAV-2cap5 (lanes 3 and 4) in the presence (lanes 1 and 3) or absence (lanes 2 and 4) 40 μM LLnL. In vitro ubiquitin conjugation to rAAV-2 or rAAV-2cap5 viral particles was performed in Panel C. 3×10⁸ particles of rAAV-2 or rAAV-2cap5 were incubated with Fraction 11 (lanes 1-7) or Fraction I and II (lanes 8-14) enzymes at 37° C. for 30 minutes or 2 hours, and then resolved on a 10% SDS-PAGE. Increased migratory size of ubiquitinated AAV capsid proteins were visualized by Western blotting with anti-AAV capsid mouse monoclonal antibody B 1 and ECL detection. The conditions for each conjugation reaction are marked below the gel.

FIG. 14. In vitro ubiquitin conjugation to rAAV-2 or rAAV-2cap5 viral particles. 3×10⁸ particles of rAAV-2 (lanes 3-6) or rAAV-2cap5 (lanes 11-14) were incubated with Fraction II or Fraction I and II enzymes at 37° C. for 2 hours, and then resolved on a 10% SDS-PAGE. Increased migratory size of ubiquitinated AAV capsid proteins was visualized by Western blotting with anti-AAV capsid mouse monoclonal antibody B1 and ECL detection. The conjugation efficiency was increased when the virus was pre-treated by heating in a boiling water bath for 10 minutes. The conditions for each conjugation reaction are marked below the gel.

FIG. 15. Luciferase activity in mouse lung 2 weeks (A) or 6 weeks (B) after infection (nasal aspiration) with AV2.RSVlucCap5 (6×10¹⁰ particles) and co-administration of Z-LLL (200 μM). For each group, n=12.

FIG. 16. Luciferase activity in mouse lung 2 weeks, 6 weeks or 3 months after infection with AV2.RSVlucCap5 and co-administration of Z-LLL (200 μM) (see FIG. 15 for details).

### DETAILED DESCRIPTION OF THE INVENTION

Definitions

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest) and/or a selectable or detectable marker.

“AAV” is adeno-associated virus, and may be used to refer to the virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its binding properties, e.g., there are six serotypes of primate AAVs, AAV-1-AAV-6, and the term encompasses pseudotypes with the same binding properties. Thus, for example, AAV-5 serotypes include AAV with the binding properties of AAV-5, e.g., a pseudotyped AAV comprising AAV-5 capsid and a rAAV genome which is not derived or obtained from AAV-5 or which genome is chimeric. The abbreviation “rAAV” refers to recombinant adeno-associated virus, also referred to as a recombinant AAV vector (or “rAAV vector”).

“Transduction” or “transducing” as used herein, are terms referring to a process for the introduction of an exogenous polynucleotide, e.g., a transgene in rAAV vector, into a host cell leading to expression of the polynucleotide, e.g., the transgene in the cell. The process includes 1) binding of the virus to the cell membrane, 2) endocytosis, 3) escape from endosomes and trafficking to the nucleus, 4) uncoating of the virus particles; 5) synthesis of the second DNA strand to form expressible double-stranded forms, including circular and linear intermediates of a monomer or a concatamer; and 6) integration into the host genome, the alteration of any of which, or a combination thereof, e.g., by an agent of the invention, results in altered expression or persistence of the introduced polynucleotide in the host cell or a population of cells. Altered expression or persistence of a polynucleotide introduced via rAAV can be determined by methods well known to the art including, but not limited to, protein expression, and DNA and RNA hybridization. The agents of the invention preferably enhance or increase viral endocytosis (Sanlioglu et al., 2001), escape from endosomes and trafficking to nucleus, and/or uncoating of the viral particles in the nucleus, so as to alter expression of the introduced polynucleotide, e.g., a transgene in a rAAV vector, in vitro or in vivo. Methods used for the introduction of the exogenous polynucleotide include well-known techniques such as transfection, lipofection, viral infection, transformation, and electroporation, as well as non-viral gene delivery techniques. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

A “detectable marker gene” is a gene that allows cells carrying the gene to be specifically detected (e.g., distinguished from cells which do not carry the marker gene). A large variety of such marker genes are known in the art.

A “selectable marker gene” is a gene that allows cells carrying the gene to be specifically selected for or against, in the presence of a corresponding selective agent. By way of illustration, an antibiotic resistance gene can be used as a positive selectable marker gene that allows a host cell to be positively selected for in the presence of the corresponding antibiotic. A variety of positive and negative selectable markers are known in the art, some of which are described below.

An “RAAV vector” as used herein refers to an AAV vector comprising a polynucleotide sequence not of AAV origin (i.e., a polynucleotide heterologous to AAV), typically a sequence of interest for the genetic transformation of a cell. In preferred vector constructs of this invention, the heterologous polynucleotide is flanked by at least one, preferably two AAV inverted terminal repeat sequences (ITRs). The term rAAV vector encompasses both rAAV vector particles and rAAV vector plasmids.

An “AAV virus” or “AAV viral particle” refers to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”. An AAV “capsid protein” includes a capsid protein of a wild-type AAV, as well as modified forms of an AAV capsid protein which are structurally and or functionally capable of packaging a rAAV genome and bind to at least one specific cellular receptor which may be different than a receptor employed by wild type AAV. A modified AAV capsid protein includes a chimeric AAV capsid protein such as one having amino acid sequences from two or more serotypes of AAV, e.g., a capsid protein formed from a portion of the capsid protein from AAV-5 fused or linked to a portion of the capsid protein from AAV-2, and a AAV capsid protein having a tag or other detectable non-AAV capsid peptide or protein fused or linked to the AAV capsid protein, e.g., a portion of an antibody molecule which binds the transferrin receptor may be recombinantly fused to the AAV-2 capsid protein.

A “helper virus” for AAV refers to a virus that allows AAV (e.g., wild-type AAV) to be replicated and packaged by a mammalian cell. A variety of such helper viruses for AAV are known in the art, including adenoviruses, herpesviruses and poxyiruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

An “infectious” virus or viral particle is one that comprises a polynucleotide component which it is capable of delivering into a cell for which the viral species is trophic. The term does not necessarily imply any replication capacity of the virus.

A “replication-competent” virus (e.g., a replication-competent AAV, sometimes abbreviated as “RCA”) refers to a phenotypically wild-type virus that is infectious, and is also capable of being replicated in an infected cell (i.e., in the presence of a helper virus or helper virus functions). In the case of AAV, replication competence generally requires the presence of functional AAV packaging genes. Preferred rAAV vectors as described herein are replication-incompetent in mammalian cells (especially in human cells) by virtue of the lack of one or more AAV packaging genes. Preferably, such rAAV vectors lack any AAV packaging gene sequences in order to minimize the possibility that RCA are generated by recombination between AAV packaging genes and an incoming rAAV vector. Preferred rAAV vector preparations as described herein are those which contain few if any RCA (preferably less than about 1 RCA per 10² rAAV particles, more preferably less than about 1 RCA per 10⁴ rAAV particles, still more preferably less than about 1 RCA per 10⁸ rAAV particles, even more preferably less than about 1 RCA per 10¹² rAAV particles, most preferably no RCA).

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A “transcriptional regulatory sequence” or “TRS,” as used herein, refers to a genomic region that controls the transcription of a gene or coding sequence to which it is operably linked. Transcriptional regulatory sequences of use in the present invention generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from that of the rest of the entity to which it is compared. For example, a polynucleotide introduced by genetic engineering techniques into a different cell type is a heterologous polynucleotide (and, when expressed, can encode a heterologous polypeptide). Similarly, a TRS or promoter that is removed from its native coding sequence and operably linked to a different coding sequence is a heterologous TRS or promoter.

A “replicon” refers to a polynucleotide comprising an origin or replication which allows for replication of the polynucleotide in an appropriate host cell. Examples of replicons include episomes (including plasmids), as well as chromosomes (such as the nuclear or mitochondrial chromosomes). “Stable integration” of a polynucleotide into a cell means that the polynucleotide has been integrated into a replicon that tends to be stably maintained in the cell. Although episomes such as plasmids can sometimes be maintained for many generations, genetic material carried episomally is generally more susceptible to loss than chromosomally integrated material. However, maintenance of a polynucleotide can often be effected by incorporating a selectable marker into or adjacent to a polynucleotide, and then maintaining cells carrying the polynucleotide under selective pressure. In some cases, sequences cannot be effectively maintained stably unless they have become integrated into a chromosome; and, therefore, selection for retention of a sequence comprising a selectable marker can result in the selection of cells in which the marker has become stably integrated into a chromosome. Antibiotic resistance genes can be conveniently employed in that regard, as is well known in the art. Typically, stably-integrated polynucleotides would be expected to be maintained on average for at least about twenty generations, preferably at least about one hundred generations, still more preferably they would be maintained permanently. The chromatin structure of eukaryotic chromosomes can influence the level of expression of an integrated polynucleotide. Having the genes carried on episomes can be particularly useful where it is desired to have multiple stably- maintained copies of a particular gene. The selection of stable cell lines having properties that are particularly desirable in the context of the present invention are described and illustrated below.

“Packaging” as used herein refers to a series of subcellular events that results in the assembly and encapsidation of a viral vector, particularly an AAV vector. Thus, when a suitable vector is introduced into a packaging cell line under appropriate conditions, it can be assembled into a viral particle. Functions associated with packaging of viral vectors, particularly AAV vectors, are described herein and in the art.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art; and illustrative uses of such sequences within the context of the present invention are provided below.

“Host cells,” “cell lines,” “cell cultures,” “packaging cell line” and other such terms denote higher eukaryotic cells, preferably mammalian cells, most preferably human cells, useful in the present invention. These cells can be used as recipients for recombinant vectors, viruses or other transfer polynucleotides, and include the progeny of the original cell that was transduced. It is understood that the progeny of a single cell may not necessarily be completely identical (in morphology or in genomic complement) to the original parent cell.

A “therapeutic gene,” “target polynucleotide,” “transgene,” “gene of interest” and the like generally refer to a gene or genes to be transferred using a vector. Typically, in the context of the present invention, such genes are located within the rAAV vector (which vector is flanked by inverted terminal repeat (ITR) regions and thus can be replicated and encapsidated into rAAV particles). Target polynucleotides can be used in this invention to generate rAAV vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; and (vi) polynucleotides for cancer therapy, such as E1A tumor suppressor genes or p53 tumor suppressor genes for the treatment of various cancers. To effect expression of the transgene in a recipient host cell, it is preferably operably linked to a promoter, either its own or a heterologous promoter. A large number of suitable promoters are known in the art, the choice of which depends on the desired level of expression of the target polynucleotide; whether one wants constitutive expression, inducible expression, cell-specific or tissue-specific expression, etc. The rAAV vector may also contain a selectable marker.

A “gene” refers to a polynucleotide containing at least one open reading frame that is capable of encoding a particular protein after being transcribed and translated.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Genetic alteration” refers to a process wherein a genetic element is introduced into a cell other than by mitosis or meiosis. The element may be heterologous to the cell, or it may be an additional copy or improved version of an element already present in the cell. Genetic alteration may be effected, for example, by transfecting a cell with a recombinant plasmid or other polynucleotide through any process known in the art, such as electroporation, calcium phosphate precipitation, or contacting with a polynucleotide-liposome complex. Genetic alteration may also be effected, for example, by transduction or infection with a DNA or RNA virus or viral vector. Preferably, the genetic element is introduced into a chromosome or mini-chromosome in the cell; but any alteration that changes the phenotype and/or genotype of the cell and its progeny is included in this term.

A cell is said to be “stably” altered, transduced or transformed with a genetic sequence if the sequence is available to perform its function during extended culture of the cell in vitro. In preferred examples, such a cell is “inheritably” altered in that a genetic alteration is introduced which is also inheritable by progeny of the altered cell.

The terms “polypeptide” and “protein” are used interchangeably herein to refer to polymers of amino acids of any length. The terms also encompass an amino acid polymer that has been modified; for example, disulfide bond formation, glycosylation, acetylation, phosphonylation, lipidation, or conjugation with a labeling component. Polypeptides such as “CFTR” and the like, when discussed in the context of gene therapy and compositions therefor, refer to the respective intact polypeptide, or any fragment or genetically engineered derivative thereof, that retains the desired biochemical function of the intact protein. Similarly, references to CFTR, and other such genes for use in gene therapy (typically referred to as “transgenes” to be delivered to a recipient cell), include polynucleotides encoding the intact polypeptide or any fragment or genetically engineered derivative possessing the desired biochemical function.

An “isolated” plasmid, virus, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment can be measured on an absolute basis, such as weight per volume of solution, or it can be measured in relation to a second, potentially interfering substance present in the source mixture. Increasing enrichments of the embodiments of this invention are increasingly more preferred. Thus, for example, a 2-fold enrichment is preferred, 10-fold enrichment is more preferred, 100-fold enrichment is more preferred, 1000-fold enrichment is even more preferred.

A preparation of AAV is said to be “substantially free” of helper virus if the ratio of infectious AAV particles to infectious helper virus particles is at least about 10²:1; preferably at least about 10⁴:1, more preferably at least about 10⁶:1; still more preferably at least about 10⁸:1. Preparations are also preferably free of equivalent amounts of helper virus proteins (i.e., proteins as would be present as a result of such a level of helper virus if the helper virus particle impurities noted above were present in disrupted form). Viral and/or cellular protein contamination can generally be observed as the presence of Coomassie staining bands on SDS gels (e.g., the appearance of bands other than those corresponding to the AAV capsid proteins VP1, VP2 and VP3).

“Efficiency” when used in describing viral production, replication or packaging refers to useful properties of the method: in particular, the growth rate and the number of virus particles produced per cell. “High efficiency” production indicates production of at least 100 viral particles per cell; preferably at least about 10,000 and more preferably at least about 100,000 particles per cell, over the course of the culture period specified.

An “individual” or “subject” treated in accordance with this invention refers to vertebrates, particularly members of a mammalian species, and includes but is not limited to domestic animals, sports animals, and primates, including humans.

“Treatment” of an individual or a cell is any type of intervention in an attempt to alter the natural course of the individual or cell at the time the treatment is initiated, e.g., eliciting a prophylactic, curative or other beneficial effect in the individual. For example, treatment of an individual may be undertaken to decrease or limit the pathology caused by any pathological condition, including (but not limited to) an inherited or induced genetic deficiency, infection by a viral, bacterial, or parasitic organism, a neoplastic or aplastic condition, or an immune system dysfunction such as autoimmunity or immunosuppression. Treatment includes (but is not limited to) administration of a composition, such as a pharmaceutical composition, and administration of compatible cells that have been treated with a composition. Treatment may be performed either prophylactically or therapeutically; that is, either prior or subsequent to the initiation of a pathologic event or contact with an etiologic agent.

The practice of the present invention will employ, unless otherwise indicated, conventional techniques of molecular biology, virology, microbiology, recombinant DNA, and immunology, which are within the skill of the art. Such techniques are explained fully in the literature. See, e.g., Sambrook, Fritsch, and Maniatis, Molecular Cloning: A Laboratory Manual, Second Edition (1989); Oligonucleotide Synthesis (M. J. Gait Ed., 1984); Animal Cell Culture (R. I. Freshney, Ed., 1987); the series Methods in Enzymology (Academic Press, Inc.); Gene Transfer Vectors for Mammalian Cells (J. M. Miller and M. P. Calos eds. 1987); Handbook of Experimental Immunology, (D. M. Weir and C. C. Blackwell, Eds.); Current Protocols in Molecular Biology (F. M. Ausubel, R. Brent, R. E. Kingston, D. D. Moore, J. G. Siedman, J. A. Smith, and K. Struhl, eds., 1987); Current Protocols in Immunology (J. E. Coligan, A. M. Kruisbeek, D. H. Margulies, E. M. Shevach and W. Strober, eds., 1991); Current Protocols in Protein Science (John E. Coligan et al., eds., Wiley and Sons, 1995); and Protein Purification: Principles and Practice (Robert K. Scopes, Springer-Verlag, 1994).

I. rAAV vectors

Recombinant AAV vectors are potentially powerful tools for human gene therapy, particularly for diseases such as cystic fibrosis and sickle cell anemia. A major advantage of rAAV vectors over other approaches to gene therapy is that they generally do not require ongoing replication of the target cell in order to become stably integrated into the host cell.

rAAV vectors and/or viruses may also contain one or more detectable markers. A variety of such markers are known, including, by way of illustration, the bacterial beta-galactosidase (lacZ) gene; the human placental alkaline phosphatase (AP) gene and genes encoding various cellular surface markers which have been used as reporter molecules both in vitro and in vivo. The rAAV vectors and/or viruses may also contain one or more selectable markers.

Recombinant AAV vectors and/or viruses can also comprise polynucleotides that do not encode proteins, including, e.g., polynucleotides encoding for antisense mRNA (the complement of mRNA) which can be used to block the translation of normal mRNA by forming a duplex with it, and polynucleotides that encode ribozymes (RNA catalysts).

II. Selection and Preparation of AAV Vector

Adeno-associated viruses of any serotype are suitable to prepare rAAV, since the various serotypes are functionally and structurally related, even at the genetic level (see, e.g., Blacklow, pp. 165-174 of Parvoviruses and Human Disease, J. R. Pattison, ed. (1988); and Rose, Comprehensive Virology, 3, 1, 1974). All AAV serotypes apparently exhibit similar replication properties mediated by homologous rep genes; and all generally bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control. Among the various AAV serotypes, AAV2 is most commonly employed.

An AAV vector of the invention typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and are preferred when it is desired that the therapeutic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or preferably in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, preferably (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present invention.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters are preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. An especially preferred sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or 1CP4; as well as vaccinia or poxyirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), preferably linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Given the relative encapsidation size limits of various AAV genomes, insertion of a large heterologous polynucleotide into the genome necessitates removal of a portion of the AAV sequence. Removal of one or more AAV genes is in any case desirable, to reduce the likelihood of generating replication-competent AAV (“RCA”). Accordingly, encoding or promoter sequences for rep, cap, or both, are preferably removed, since the functions provided by these genes can be provided in trans.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are preferably not flanked by AAV ITRs and preferably do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this invention in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids, AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., WO 95/13392); Burstein et al. (WO 98/23018); and Johnson et al. (U.S. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this invention.

III. Generating rAAV

To generate recombinant AAV particles useful for such purposes as gene therapy, the packaging cell line is preferably supplied with a recombinant AAV vector comprising AAV inverted terminal repeat (ITR) regions surrounding one or more polynucleotides of interest (or “target” polynucleotides).

The target polynucleotide is generally operably linked to a promoter, either its own or a heterologous promoter. A large number of suitable promoters are known in the art, the choice of which depends on the desired level of expression of the target polynucleotide (i.e., whether one wants constitutive expression, inducible expression, cell-specific or tissue-specific expression, etc.).

Preferably, the rAAV vector also contains a positive selectable marker in order to allow for selection of cells that have been infected by the rAAV vector. Negative selectable markers can also be included; as a means of selecting against those same cells should that become necessary or desirable. In a preferred embodiment, one can make use of the “bifunctional selectable fusion genes” described by S. D. Lupton; see, e.g., PCT/US91/08442 and PCT/US94/05601. Briefly, those constructs involve direct translational fusions between a dominant positive selectable marker and a negative selectable marker. Preferred positive selectable markers are derived from genes selected from the group consisting of hph, neo, and gpt, and preferred negative selectable markers are derived from genes selected from the group consisting of cytosine deaminase, HSV-I TK, VZV TK, HPRT, APRT and gpt. Especially preferred markers are bifunctional selectable fusion genes wherein the positive selectable marker is derived from hph or neo, and the negative selectable marker is derived from cytosine deaminase or a TK gene.

Useful target polynucleotides can be employed in rAAV vectors for a number of different applications. Such polynucleotides include, but are not limited to: (i) polynucleotides encoding proteins useful in other forms of gene therapy to relieve deficiencies caused by missing, defective or sub-optimal levels of a structural protein or enzyme; (ii) polynucleotides that are transcribed into anti-sense molecules; (iii) polynucleotides that are transcribed into decoys that bind transcription or translation factors; (iv) polynucleotides that encode cellular modulators such as cytokines; (v) polynucleotides that can make recipient cells susceptible to specific drugs, such as the herpes virus thymidine kinase gene; and (vi) polynucleotides for cancer therapy, such as the wild-type p53 tumor suppressor cDNA for replacement of the missing or damaged p53 gene associated with some lung and breast cancers, or the EIA tumor suppressor gene which is capable of inhibiting tumorigenesis and/or metastasis of a variety of different cancers including breast and ovarian cancers.

Since the therapeutic specificity of the resulting recombinant AAV particle is determined by the particular vector or pro-vector introduced, the same basic packaging cell line can be modified for any of these applications. For example, a vector comprising a specific target polynucleotide can be introduced into the packaging cell for production of the AAV vector by any of several possible methods; including, for example, electroporation or transfection of a plasmid comprising an rAAV pro-vector, or infection with an rAAV or helper virus comprising an rAAV vector or pro-vector.

Helper virus can be introduced before, during or after introduction of the rAAV vector. For example, the plasmid can be co-infected into the culture along with the helper virus; and the cells can then be cultured for a sufficient period, typically 2-5 days, in conditions suitable for replication and packaging as known in the art (see references above and examples below). Lysates are prepared, and the recombinant AAV vector particles are purified by techniques known in the art.

In a preferred embodiment, also illustrated in the Examples below, a recombinant AAV vector is itself stably integrated into a mammalian cell to be used for packaging. Such rAAV “producer cells” can then be grown and stored until ready for use. To induce production of rAAV particles from such producer cells, the user need only infect the cells with helper virus and culture the cells under conditions suitable for replication and packaging of AAV (as described below).

Alternatively, one or more of the AAV split-packaging genes or the rAAV vector can be introduced as part of a recombinant helper virus. For example, the E1, E3 and/or the E4 genes of adenovirus can be replaced with one or more split-packaging genes or an rAAV vector. Techniques for facilitating cloning into adenovirus vectors, e.g., into the E1 and/or E3 regions, are known in the art (see, e.g., Bett, A. J. et al., Proc. Natl. Acad. Sci. USA, 91, 8802-8806 (1994)). Thus, a helper virus such as a recombinant adenovirus, can be used to provide helper virus functions as well as AAV packaging genes and/or an rAAV pro-vector, since (as is known in the art) a number of genes in such a helper virus (e.g., the E3 gene of adenovirus) can be replaced without eliminating helper virus activity. Additional genes can be inserted into such a helper virus by providing any necessary helper virus functions in trans. For example, human 293 cells contain adenoviral genes that can complement adenoviral E1 mutants. Thus, heterologous genes can also be cloned into an adenovirus in which the E1 genes have been deleted, for use in cells that can effectively provide such adenoviral functions in trans. Alternatively, the use of a helper virus can be eliminated by providing all necessary helper virus functions in the packaging cell.

IV. Introduction of Genetic Material Into Cells

As is described in the art, and illustrated both herein and in the references cited above, genetic material can be introduced into cells (such as mammalian “producer” cells for the production of AAV) using any of a variety of means to transform or transduce such cells. By way of illustration, such techniques include, for example, transfection with bacterial plasmids, infection with viral vectors, electroporation, calcium phosphate precipitation, and introduction using any of a variety of lipid-based compositions (a process often referred to as “lipofection”). Methods and compositions for performing these techniques have been described in the art and are widely available.

Selection of suitably altered cells may be conducted by any technique in the art. For example, the polynucleotide sequences used to alter the cell may be introduced simultaneously with or operably linked to one or more detectable or selectable markers as is known in the art. By way of illustration, one can employ a drug-resistance gene as a selectable marker. Drug-resistant cells can then be picked and grown, and then tested for expression of the desired sequence, i.e., a packaging gene product, or a product of the heterologous polynucleotide, as appropriate. Testing for acquisition, localization and/or maintenance of an introduced polynucleotide can be performed using DNA hybridization-based techniques (such as Southern blotting and other procedures as is known in the art). Testing for expression can be readily performed by Northern analysis of RNA extracted from the genetically altered cells, or by indirect immunofluorescence for the corresponding gene product. Testing and confirmation of packaging capabilities and efficiencies can be obtained by introducing to the cell the remaining functional components of AAV and a helper virus, to test for production of AAV particles. Where a cell is inheritably altered with a plurality of polynucleotide constructs, it is generally more convenient (though not essential) to introduce them to the cell separately, and validate each step seriatim. References describing such techniques include those cited herein.

V. Selection and Preparation of Helper Virus

As discussed above, AAV is a parvovirus that is defective for self-replication, and must generally rely on a helper virus to supply certain replicative functions. A number of such helper viruses have been identified, including adenoviruses, herpes viruses (including but not limited to HSV1, cytomegalovirus and HHV-6), and pox viruses (particularly vaccinia). Any such virus may be used with this invention.

Frequently, the helper virus is an adenovirus of a type and subgroup that can infect the intended host cell. Human adenovirus of subgroup C, particularly serotypes 1, 2, 4, 6, and 7, are commonly used. Serotype 5 is generally preferred.

The features and growth patterns of adenovirus are known in the art. The reader may refer, for example, to Horowitz, “Adenoviridae and their replication,” pp. 771-816 in Fundamental Virology, Fields et al., eds. The packaged adenovirus genome is a linear DNA molecule, linked through adenovirus ITRs at the left- and right-hand termini through a terminal protein complex to form a circle. Control and encoding regions for early, intermediate, and late components overlap within the genome. Early region genes are implicated in replication of the adenovirus genome, and are grouped depending on their location into the E1, E2, E3, and E4 regions.

Although not essential, in principle it is desirable that the helper virus strain be defective for replication in the subject ultimately to receive the genetic therapy. Thus, any residual helper virus present in an rAAV preparation will be replication-incompetent. Adenoviruses from which the E1A or both the E1A and the E3 region have been removed are not infectious for most human cells. They can be replicated in a permissive cell line (e.g., the human 293 cell line) which is capable of complementing the missing activity. Regions of adenovirus that appear to be associated with helper function, as well as regions that do not, have been identified and described in the art (see, e.g., P. Colosi et al., WO97/17458, and references cited therein).

VI. Uses of rAAV for Gene Therapy

AAV vectors can be used for administration to an individual for purposes of gene therapy. Suitable diseases for gene therapy include but are not limited to those induced by viral, bacterial, or parasitic infections, various malignancies and hyperproliferative conditions, autoimmune conditions, and congenital deficiencies.

Gene therapy can be conducted to enhance the level of expression of a particular protein either within or secreted by the cell. Vectors of this invention may be used to genetically alter cells either for gene marking, replacement of a missing or defective gene, or insertion of a therapeutic gene. Alternatively, a polynucleotide may be provided to the cell that decreases the level of expression. This may be used for the suppression of an undesirable phenotype, such as the product of a gene amplified or overexpressed during the course of a malignancy, or a gene introduced or overexpressed during the course of a microbial infection. Expression levels may be decreased by supplying a therapeutic polynucleotide comprising a sequence capable, for example, of forming a stable hybrid with either the target gene or RNA transcript (antisense therapy), capable of acting as a ribozyme to cleave the relevant mRNA or capable of acting as a decoy for a product of the target gene.

The introduction of rAAV vectors by the methods of the present invention may involve use of any number of delivery techniques (both surgical and non-surgical) which are available and well known in the art. Such delivery techniques, for example, include vascular catheterization, cannulization, injection, inhalation, inunction, topical, oral, percutaneous, intra-arterial, intravenous, and/or intraperitoneal administrations. Vectors can also be introduced by way of bioprostheses, including, by way of illustration, vascular grafts (PTFE and dacron), heart valves, intravascular stents, intravascular paving as well as other non-vascular prostheses. General techniques regarding delivery, frequency, composition and dosage ranges of vector solutions are within the skill of the art.

In particular, for delivery of a vector of the invention to a tissue, any physical or biological method that will introduce the vector to a host animal can be employed. Vector means both a bare recombinant vector and vector DNA packaged into viral coat proteins, as is well known for AAV administration. Simply dissolving an AAV vector in phosphate buffered saline has been demonstrated to be sufficient to provide a vehicle useful for muscle tissue expression, and there are no known restrictions on the carriers or other components that can be coadministered with the vector (although compositions that degrade DNA should be avoided in the normal manner with vectors). Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The vectors can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of the AAV vector as a free acid (DNA contains acidic phosphate groups) or a pharmacologically acceptable salt can be prepared in water suitably mixed with a surfactant such as hydroxypropylcellulose. A dispersion of AAV viral particles can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the AAV vector in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration), otherwise similar to the above parenteral solutions, are prepared in containers suitable for incorporation into a transdermal patch, and can include known carriers, such as pharmaceutical grade dimethylsulfoxide (DMSO).

Of particular interest is the correction of the genetic defect of cystic fibrosis, by supplying a properly functioning cystic fibrosis transmembrane conductance regulator (CFTR) to the airway epithelium. Thus, rAAV vectors encoding native CFTR protein, and mutants and fragments thereof, are all preferred embodiments of this invention.

Compositions of this invention may be used in vivo as well as ex vivo. In vivo gene therapy comprises administering the vectors of this invention directly to a subject. Pharmaceutical compositions can be supplied as liquid solutions or suspensions, as emulsions, or as solid forms suitable for dissolution or suspension in liquid prior to use. For administration into the respiratory tract, a preferred mode of administration is by aerosol, using a composition that provides either a solid or liquid aerosol when used with an appropriate aerosolubilizer device. Another preferred mode of administration into the respiratory tract is using a flexible fiberoptic bronchoscope to instill the vectors. Typically, the viral vectors are in a pharmaceutically suitable pyrogen-free buffer such as Ringer's balanced salt solution (pH 7.4). Although not required, pharmaceutical compositions may optionally be supplied in unit dosage form suitable for administration of a precise amount.

An effective amount of virus is administered, depending on the objectives of treatment. An effective amount may be given in single or divided doses. Where a low percentage of transduction can cure a genetic deficiency, then the objective of treatment is generally to meet or exceed this level of transduction. In some instances, this level of transduction can be achieved by transduction of only about 1 to 5% of the target cells, but is more typically 20% of the cells of the desired tissue type, usually at least about 50%, preferably at least about 80%, more preferably at least about 95%, and even more preferably at least about 99% of the cells of the desired tissue type. As a guide, the number of vector particles present in a single dose given by bronchoscopy will generally be at least about 1×10⁸, and is more typically 5×10⁸, 1×10¹⁰, and on some occasions 1×10¹¹ particles, including both DNAse-resistant and DNAse-susceptible particles. In terms of DNAse-resistant particles, the dose will generally be between 1×10⁶ and 1×10¹⁴ particles, more generally between about 1×10⁸ and 1×10¹² particles. The treatment can be repeated as often as every two or three weeks, as required, although treatment once in 180 days may be sufficient.

To confirm the presence of the desired DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence of a polypeptide expressed from a gene present in the vector, e.g., by immunological means (immunoprecipitations, immunoaffinity columns, ELISAs and Western blots) or by any other assay useful to identify the presence and/or expression of a particular nucleic acid molecule failing within the scope of the invention.

To detect and quantitate RNA produced from introduced DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the DNA segment in question, they do not provide information as to whether the DNA segment is being expressed. Expression may be evaluated by specifically identifying the polypeptide products of the introduced DNA sequences or evaluating the phenotypic changes brought about by the expression of the introduced DNA segment in the host cell.

Thus, the effectiveness of the genetic alteration can be monitored by several criteria. Samples removed by biopsy or surgical excision may be analyzed by in situ hybridization, PCR amplification using vector-specific probes, RNAse protection, immunohistology, or immunofluorescent cell counting. When the vector is administered by bronchoscopy, lung function tests may be performed, and bronchial lavage may be assessed for the presence of inflammatory cytokines. The treated subject may also be monitored for clinical features, and to determine whether the cells express the function intended to be conveyed by the therapeutic polynucleotide.

The decision of whether to use in vivo or ex vivo therapy, and the selection of a particular composition, dose, and route of administration will depend on a number of different factors, including but not limited to features of the condition and the subject being treated. The assessment of such features and the design of an appropriate therapeutic regimen is ultimately the responsibility of the prescribing physician.

The foregoing description provides, inter alia, methods for generating high titer preparations of recombinant AAV vectors that are substantially free of helper virus (e.g., adenovirus) and cellular proteins. It is understood that variations may be applied to these methods by those of skill in this art without departing from the spirit of this invention.

VII. Agents Useful in the Practice of the Invention

Agents useful in the practice of the invention include agents which alter rAAV transduction efficiency. Preferred agents are those which enhance or increase rAAV transduction. Such agents include agents which enhance viral endocytosis, e.g., brefeldin A, endosomal processing and/or trafficking to the nucleus, e.g., cysteine protease inhibitors. Preferably, the inhibitors are endosomal, e.g., lysosomal, cysteine protease inhibitors. More preferably, the agents of the invention are reversible cysteine protease inhibitors. Cysteine protease inhibitors within the scope of the invention include the cystatins, e.g., cystatin B or cystatin C, antipain, leupeptin, E-64, E-64c, E-64d, K02 (Wacher et al., J. Pharma. Sci., 87, 1322 (1998)), LLnL, Z-LLL, CBZ-Val-Phe-H, cysteine protease inhibitors such as those disclosed in U.S. Pat. Nos. 5,607,831, 5,374,623, 5,639,732, 5,658,906, 5,714,484, 5,560,937, 5,374,623, 5,607,831, 5,723,580, 5,744,339, 5,827,877, 5,852,007, and 5,776,718, JP 10077276, JP 8198870, JP 8081431, JP 7126294, JP 4202170, WO 96/21006 and WO 96/40737.

Preferred cysteine protease inhibitors are peptides or analogs thereof. Preferred peptide cysteine protease inhibitors within the scope of the invention comprise 2 to 20, more preferably 3 to 10, and even more preferably 3 to 8, amino acid residues. “Amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, a-methyl-alanine, para-benzoylphenylalanine, phenylglycine, propargylglycine, sarcosine, nor-leucine, nor-valine, and tert-butylglycine). Peptide analogs are molecules which comprise at least one amino acid in D form and/or an unnatural amino acid, or other moiety which is not a natural amino acid.

Preferred peptide cysteine protease inhibitors include a compound of formula (I): R₁-A-(B)ₙ-C wherein R₁ is an N-terminal amino acid blocking group; each A and B is independently an amino acid; C is an amino acid wherein the terminal carboxy group has been replaced by a CHO group; and n is 0, 1, 2, or 3; or a pharmaceutically acceptable salt thereof. In one preferred embodiment, R₁ is (C₁-C₁₀)alkanoyl, acetyl or benzyloxycarbonyl. In another preferred embodiment, each A and B is independently alanine, arginine, glycine, isoleucine, leucine, valine, nor-leucine or nor-valine, and more preferably each A and B is isoleucine. In yet another preferred embodiment, C is alanine, arginine, glycine, isoleucine, leucine, valine, nor-leucine or nor-valine, wherein the terminal carboxy group has been replaced by a CHO group, and more preferably, C is nor-leucine or nor-valine, wherein the terminal carboxy group has been replaced by a CHO group.

In a further preferred embodiment, R₁ is (C₁-C₁₀)alkanoyl or benzyloxycarbonyl; A and B are each isoleucine; C is nor-leucine or nor-valine, wherein the terminal carboxy group has been replaced by a CHO group; and N is 1.

Also included within the scope of the invention is a compound of formula (II):

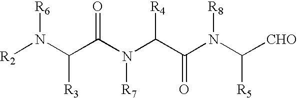

wherein

R₂ is an N-terminal amino acid blocking group;

R₃, R₄, and R₅ are each independently hydrogen, (C₁-C₁₀)alkyl, aryl or aryl(C₁-C₁₀)alkyl; and

R₆, R₇, and R₈ are each independently hydrogen, (C₁-C₁₀)alkyl, aryl or aryl(C₁-C₁₀)alkyl; or a pharmaceutically acceptable salt thereof. Preferably, R₂ is (C₁-C₁₀)alkanoyl, acetyl or benzyloxycarbonyl. Also preferably, R₃ is hydrogen or (C₁-C₁₀)alkyl, e.g., 2-methylpropyl. It is preferred that R₄ is hydrogen or (C₁-C₁₀)alkyl, e.g., 2-methylpropyl.

In another preferred embodiment, R₅ is hydrogen or (C₁-C₁₀)alkyl, for example, butyl or propyl.

In a further preferred embodiment, R₂ is acetyl or benzyloxycarbonyl; R₃ and R₄ are each 2-methylpropyl; R₅ is butyl or propyl; and R₆, R₇, and R₈ are each independently hydrogen.

Another preferred agent useful in the methods of the invention is a compound of formula (III):

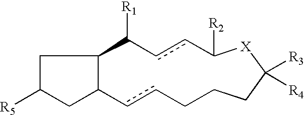

wherein

R₁ is H, halogen, (C₁-C₁₀)alkyl, (C₁-C₁₀)alkenyl, (C₁-C₁₀)alkynyl, (C₁-C₁₀)alkoxy, (C₁-C₁₀)alkanoyl, (═O), (═S), OH, SR, CN, NO₂, trifluoromethyl or (C₁-C₁₀)alkoxy, wherein any alkyl, alkenyl, alkynyl, alkoxy or alkanoyl may optionally be substituted with one or more halogen, OH, SH, CN, NO₂, trifluoromethyl, NRR or SR, wherein each R is independently H or (C₁-C₁₀)alkyl;

R₂ is (═O) or (═S);

R₃ is H, (C₁-C₁₀)alkyl, (C₁-C₁₀)alkenyl, (C₁-C₁₀)alkynyl, (C₁-C₁₀)alkoxy or (C₃-C₈)cycloalkyl, wherein any alkyl, alkenyl, alkynyl, alkoxy or cycloalkyl may optionally be substituted with one or more halogen, OH, CN, NO₂, trifluoromethyl, SR, or NRR, wherein each R is independently H or (C₁-C₁₀)alkyl;

R₄ is H, (C₁-C₁₀)alkyl, (C₁-C₁₀)alkenyl, (C₁-C₁₀)alkynyl, (C₁-C₁₀)alkoxy or (C₃-C₈)cycloalkyl, wherein any alkyl, alkenyl, alkynyl, alkoxy or cycloalkyl may optionally be substituted with one or more halogen, OH, CN, NO₂, trifluoromethyl, SR, or NRR, wherein each R is independently H or (C₁-C₁₀)alkyl;

R₅ is H, halogen, (C₁-C₁₀)alkyl, (C₁-C₁₀)alkenyl, (C₁-C₁₀)alkynyl, (C₁-C₁₀)alkoxy, (C₁-C₁₀)alkanoyl, (═O), (═S), OH, SR, CN, NO₂ or trifluoromethyl, wherein any alkyl, alkenyl, alkynyl, alkoxy or alkanoyl may optionally be substituted with one or more halogen, OH, SH, CN, NO₂, trifluoromethyl, NRR or SR, wherein each R is independently H or (C₁-C₁₀)alkyl; and

X is O, S or NR wherein R is H or (C₁-C₁₀)alkyl, or a pharmaceutically acceptable salt thereof.

The following definitions apply unless otherwise stated. Alkyl denotes a straight or a branched group, but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic.

Suitable N-amino acid blocking groups are known to those skilled in the art (See, for example, T. W. Greene, Protecting Groups In Organic Synthesis; Wiley: New York, 1981, and references cited therein). Preferred values for R₁ include (C₁-C₁₀)alkanoyl (e.g. acetyl) and benzyloxycarbonyl.

VIII. Dosages, Formulations and Routes of Administration of the Agents of the Invention

Administration of the agents identified in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. When the agents of the invention are employed for prophylactic purposes, agents of the invention are amenable to chronic use, preferably by systemic administration.

The agents of the invention, including a compound of formula (I), (II), (III), or (IV) including their salts, are preferably administered at dosages of about 0.01 μM to about 1 mM, more preferably about 0.1 μM to about 40 μM, and even more preferably, about 1 μM to 40 μM, although other dosages may provide a beneficial effect. For example, preferred dosages of LLnL include about 1 μM to 40 μM while preferred dosages of Z-LLL include 0.1 μM to about 4 μM.

One or more suitable unit dosage forms comprising the agents of the invention, which, as discussed below, may optionally be formulated for sustained release, can be administered by a variety of routes including oral, or parenteral, including by rectal, transdermal, subcutaneous, intravenous, intramuscular, intraperitoneal, intrathoracic, intrapulmonary and intranasal routes. For example, for administration to the liver, intravenous administration is preferred. For administration to the lung, airway administration is preferred. The formulations may, where appropriate, be conveniently presented in discrete unit dosage forms and may be prepared by any of the methods well known to pharmacy. Such methods may include the step of bringing into association the agent with liquid carriers, solid matrices, semi-solid carriers, finely divided solid carriers or combinations thereof, and then, if necessary, introducing or shaping the product into the desired delivery system.

When the agents of the invention are prepared for oral administration, they are preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Pharmaceutical formulations containing the agents of the invention can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The agents of the invention can also be formulated as elixirs or solutions for convenient oral administration or as solutions appropriate for parenteral administration, for instance by intramuscular, subcutaneous or intravenous routes.

The pharmaceutical formulations of the agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and a-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosage forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The agents of the invention can be delivered via patches for transdermal administration. See U.S. Pat. No. 5,560,922 for examples of patches suitable for transdermal delivery of an agent. Patches for transdermal delivery can comprise a backing layer and a polymer matrix which has dispersed or dissolved therein an agent, along with one or more skin permeation enhancers. The backing layer can be made of any suitable material which is impermeable to the agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogel), polyvinylidene chloride, poly(ethylene terephthalate), butyl rubber, epichlorohydrin rubbers, ethylenvinyl alcohol copolymers, ethylene-vinyloxyethanol copolymers; silicone copolymers, for example, polysiloxane-polycarbonate copolymers, polysiloxanepolyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Agents released from a transdermal delivery system must be capable of penetrating each layer of skin. In order to increase the rate of permeation of an agent, a transdermal drug delivery system must be able in particular to increase the permeability of the outermost layer of skin, the stratum corneum, which provides the most resistance to the penetration of molecules. The fabrication of patches for transdermal delivery of agents is well known to the art.

For administration to the upper (nasal) or lower respiratory tract by inhalation, the agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the agent may be administered via nose drops, a liquid spray, such as via a plastic bottle atomizer or metered-dose inhaler. Typical of atomizers are the Mistometer (Wintrop) and the Medihaler (Riker).

The local delivery of the agents of the invention can also be by a variety of techniques which administer the agent at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the agents may be formulated as is known in the art for direct application to a target area. Conventional forms for this purpose include wound dressings, coated bandages or other polymer coverings, ointments, creams, lotions, pastes, jellies, sprays, and aerosols. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of an agent of the invention present in a topical formulation will depend on various factors, but generally will be from 0.01% to 95% of the total weight of the formulation, and typically 0.1-25% by weight.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The formulations and compositions described herein may also contain other ingredients such as antimicrobial agents, or preservatives. Furthermore, the active ingredients may also be used in combination with other agents, for example, bronchodilators.

The agents of this invention may be administered to a mammal alone or in combination with pharmaceutically acceptable carriers. As noted above, the relative proportions of active ingredient and carrier are determined by the solubility and chemical nature of the compound, chosen route of administration and standard pharmaceutical practice.

The dosage of the present agents will vary with the form of administration, the particular compound chosen and the physiological characteristics of the particular patient under treatment. Generally, small dosages will be used initially and, if necessary, will be increased by small increments until the optimum effect under the circumstances is reached.

The invention will be further described by, but is not limited to, the following examples.

### EXAMPLE 1

### Enhancement of Muscle Gene Delivery with Pseudotyped AAV-5 Correlates with Myoblast Differentiation

To better understand the mechanisms responsible for increased transduction of rAAV-5 in muscle, muscle transduction of a pseudotyped virus was evaluated in which rAAV-2 genomes were packaged in AAV-5 capsids (rAAV-2cap5). This hybrid virus should retain the well-established molecular characteristics of the AAV-2 genome, hence allowing for direct determination of the influence of the capsid on the efficiency of rAAV gene delivery to muscle. As described below, an in vitro study in myoblasts and in vivo study in muscle demonstrated that gene delivery by pseudotyped rAAV-2cap5 virus was greatly enhanced over rAAV-2 vectors in differentiated myofibers but not in undifferentiated myoblasts. Interestingly, the enhancement in gene transfer with rAAV-2cap5 virus did not completely correlate with increased viral binding, suggesting that a post-entry processing event is likely affected by the different capsid structures of AAV-2 and AAV-5. These findings suggest that the intracellular processing of rAAV-2 might also represent a partial barrier to rAAV-2 transduction in muscle, as is seen in other tissues such as the airway.

Materials and Methods

Recombinant AAV Production. rAAV-2 virus expressing EGFP was generated using the pcisGFPori3 proviral plasmid (Duan et al., 1998). The proviral plasmid pcis RSV.Luciferase, having the RSV promoter driving the luciferase gene, was generated by two-step cloning. First, a 1 kb blunted SalI fragment from pREP4 (Invitrogen) was inserted into the blunted XbaI backbone of pSub20l to generate pDD5 (Samulski et al., 1987). Second, a 1.7 kb KpnI/XbaI fragment from pGL3Basic (Promega) was inserted into KpnI/NheI site in pDD5 to generate pcis RSV.Luciferase. Two helper plasmids (pAV5-Trans and pAV2-Rep) were used to package the AAV-2 genome into the AAV-5 capsid (Yan et al., 2001). Briefly, the AAV-5 coding regions (Cap and Rep) were amplified from AAV-5 viral DNA using PCR (Bantel-Schaal et al., 1999). pAV5-Trans was generated by replacing AAV-2 Cap and Rep genes in pAAV/Ad with a 4.3 kb fragment containing the AAV-5 Cap and Rep genes (Samulski et al., 1989). pAV2-Rep was generated by deleting the AAV-2 Cap gene in pAAV/Ad (Samulski et al., 1989).

rAAV-2 viral stocks were prepared according to a three plasmid transfection adenovirus-free protocol described in Xiao et al. (1998). Briefly, 60% confluent 293 cells were co-transfected with a proviral plasmid (pcisEGFPori3 or pcisRSV-luciferase), AAV-2 helper plasmid (pXX-2), and adenoviral helper plasmid (pXX6-80) in a ratio of 1:1:3 (Duan et al., 1987). The crude viral lysate was purified on a Poros heparin column (PerSeptive, Applied Biosystems) using a Beckman Biosys 2000 HPLC Workstation and a linear NaCl gradient. The dominant A₂₈₀ peak fractions (AAV fractions) were pooled and dialyzed against HEPES buffer (20 mM Hepes, 150 mM NaCl, pH 7.8), and stored in aliquots at −80° C. in 5% glycerol. Typical yields were approximately 5×10¹² DNA particles for a twenty 150 mm plate preparation. Contamination with wild-type AAV-2 was determined as described in Yan et al. (2000) and was less than one functional particle per 1×10¹⁰ rAAV particles.

Pseudotyped rAAV-2cap5 virus (rAAV-2 genomes packaged in AAV-5 capsids) were generated using a modified adenovirus-free system. Briefly, 60% confluent 293 cells were cotransfected with the proviral plasmid (pcisEGFPori3 or pcis RSV-luciferase), AAV-2 Rep plasmid (pAV2-Rep), AAV-5 helper plasmid (pAV5-Trans) and adenoviral helper plasmid (pXX6-80) in a ratio of 1:1:1:3. Crude viral lysate was purified through three rounds of CsCl equilibrium isopycnic centrifugation for rAAV-2 as described in Duan et al. (1997). Typical yields from this preparation were approximately 5×10² DNA particle for a twenty 150 mm plate preparation. The physical titer of the viral stock was determined by slot blot hybridization against plasmid standards as described in Duan et al. (1997). Wild type (wt) AAV-2/5 hybrid contamination was evaluated by DNA PCR for Rep and Cap genes. Briefly, the viral stock was digested with Proteinase K at 37° C. for 30 minutes. Nested PCR was then performed using AAV-5 Cap and Rep gene specific primer sets. Less than one particle of the wt hybrid virus was detected in 1×10¹⁰ pseudotyped viral particles (limits of sensitivity) as determined against plasmid Rep and Cap standards.

To confirm that encapsidation of rAAV-2 genome in AAV-5 capsid did not alter the molecular characteristics of the rAAV-2 genome, several control experiments were performed using AAV carrying the CMV-EGFP expression cassette. First, induction of Rfm (replication form monomer) and Rfd (replication form dimer) were equivalent for both rAAV-2 and rAAV-2cap5 virus in the presence of Ad.d1802 co-infection (data not shown). Ad.d1802 co-infection also induced EGFP expression from rAAV-2 and rAAV-2cap5 virus to a similar extent. Second, using a previously described bacterial rescue assay (Duan et al., 1998), circular monomers and multimers with similar molecular structures were identified in Hela cells infected with either rAAV-2 or rAAV-2cap5 virus (data not shown).

Recombinant AAV transduction in C2C12 cells. The C2C12 muscle cell line was obtained from ATCC (Catalog number CRL-1772). The cells were cultured in 10% FBS (fetal bovine serum), 100 U/ml penicillin G and 100 μg/ml streptomycin DMEM (Dulbecco's Modified Eagle Medium) and maintained in 37° C. incubator at 5% CO₂. Differentiation was induced by culturing the cells in 10% horse serum (Yatte et al., 1997). Infections were performed in serum-free DMEM for the indicated amount of time specified in each experiment. When required, 20% FBS DMEM was added 2 hours after infection to bring final serum level to 10%. In the case of heparin competition experiments, viruses were preincubated with 20 μg/ml of free heparin (Sigma) for 60 minutes on ice and infections were then carried out in serum-free medium containing 20 μg/ml free heparin (final concentration) (Walters et al., 2000). To study the effect of sialic acid on rAAV binding, C2C12 cells were first rinsed with serum-free DMEM and then incubated with Type III neuramimidase (sialidase) (Sigma-Aldrich, catalog number N7885) at a final enzyme concentration of 200 mU/ml in serum-free medium for 2 hours at 37° C. The C2C12 cells were then washed with serum-free DMEM before viral inoculation (Pikcles et al., 2000; and Walters et al., 2001).

To analyze the effect of the proteasome inhibitor on rAAV transduction, indicated amount of viral particles were applied to the C2C12 cells in the presence or absence of proteasome inhibitors in serum-free medium. Tripeptide proteasome inhibitors N-Acetyl-L-Leucyl-L-Leucyl-Norleucine (LLnL) and benzyloxycarbonyl-Leu-Leu-1-leucinal (Z-LLL) were purchased from Calbiochem-Novabiochem Corporation (La Jolla, Calif.). At one hour post-infection, the final serum concentration was increased to 10% by the additional FBS. Both virus and proteasome inhibitors were removed from cells at 4 hours post-infection. Transgene expression was quantified at 24 hours post-infection.

Analysis of rAAV transduction in C2C12 cells. The efficiency of rAAV transduction in C2C 12 cells was monitored by the level of EGFP or luciferase transgene expression. EGFP expression was monitored by fluorescence microscopy and luciferase expression was determined using a protocol described in Duan et al. (2000a) at a measuring sensitivity of 75%. To evaluate viral binding and persistence in C2C 12 cells, the low molecular weight Hirt DNA was harvested at the indicated times following viral infection. DNA samples were then resolved in a 0.8% agarose gel and blotted on to Hybond N+nylon membrane as described in Duan et al. (1999). Each lane represents the DNA from one 35 mm plate cell culture. The viral genomes were detected with a transgene specific probe at 106 cpm/ml and washed at a stringency of 0.1×SSC, 0.1% SDS at 60° C. for 20 minutes.

Detection of alpha-2,3 linked sialic acid expression in C2C12 cells. C2C12 cells were plated on sterile positively-charged glass slides at a concentration of 2×10⁵ cells/slide and differentiation was induced as described above. MAL II lectin binding assays were performed by first chilling the cells at 4° C. for 10 minutes in serum-free media. The cultures were then incubated with biotinylated MAL II (Vector Laboratories Inc. Catalog number B-1265) at 4° C. for 30 minutes. After three washes with serum-free DMEM, cells were fixed with 4% paraformaldehyde in phosphate buffed saline (PBS). Following fixation, the cells were rinsed with HEPES buffer and then incubated with fluorescence isothiocyanate (FITC) conjugated-avidin at room temperature for 15 minutes. Finally, cells were mounted with Citifluo antifadent and the amount of cell surface alpha-2,3 linked sialic acid was determined by indirect fluorescent microscopy.

Evaluating rAAV transduction in murine skeletal muscle. Snj/ScSn mice were purchased from Jackson Laboratory. Snj mice are a normal BL10 strain. ScSn mice (mdx) have a spontaneous mutation in exon 23 of the dystrophin gene and do not express murine dystrophin (Bulfield et al., 1984). Since the dystrophic phenotype is manifested only in adult mice, 6-month-old mice were employed. The delivery of rAAV to the anterior tibialis was performed according Duan et al. (1998). To decrease inter-mouse variability, the left anterior tibialis muscle of each mouse was infected with 2×10¹⁰ particles rAAV-2cap5 virus, and the right anterior tibialis muscle of the same mouse was infected with 2×10¹⁰ particles rAAV-2. EGFP expression was determined either in freshly isolated muscles or in 15 μm cryosections from paraformaldehyde fixed tissues. To visualize the pathologic changes in mdx mouse muscle, mice were infused with 400 μl of Evans blue dye (10 mg/ml) through tail vein at 5 hours prior to tissue harvest. To facilitate contraction induced muscle injury and dye diffusion, mice were exercised by swimming twice for 10 minutes at 30 minute intervals during the first hour following dye injection. Muscle luciferase levels, following infection with 2×10¹⁰ particles per muscle of luciferase expressing rAAV-2 or rAAV-2cap5, were analyzed as described Duan et al. (1998).

Results

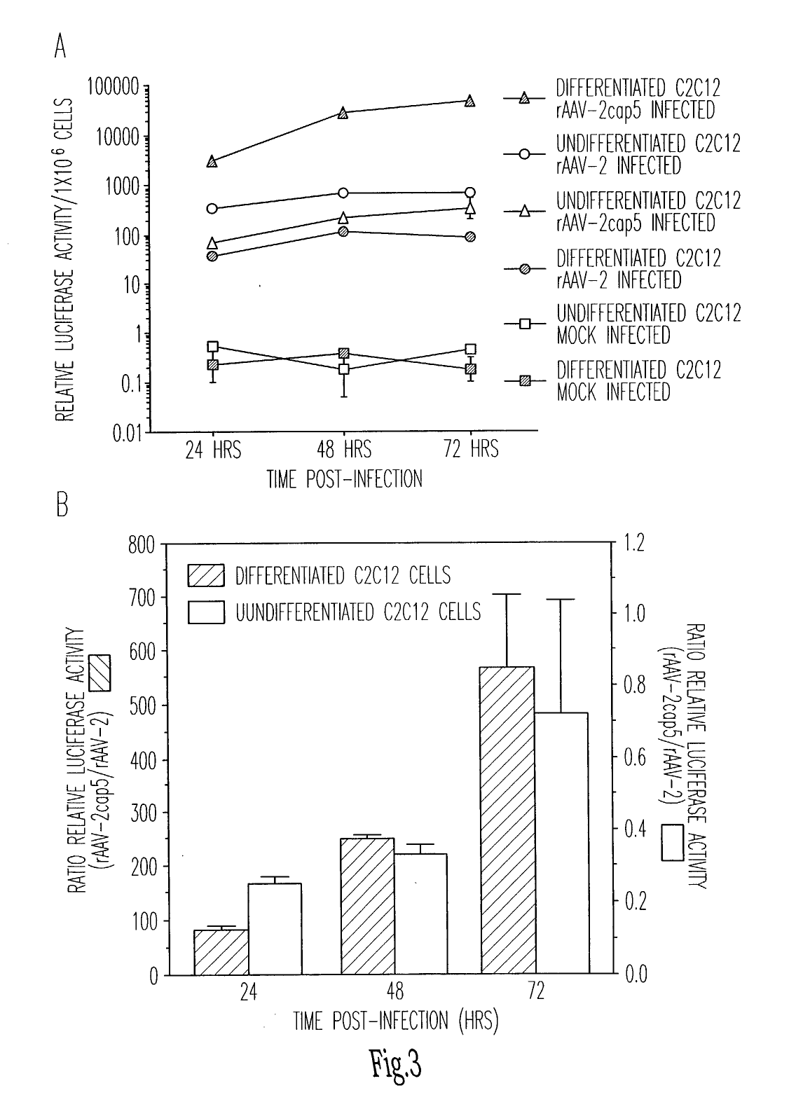

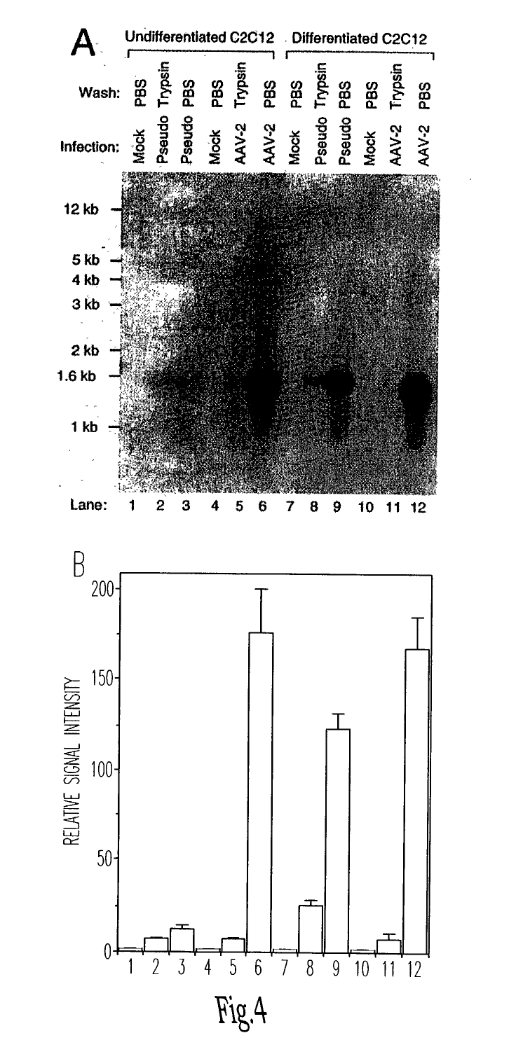

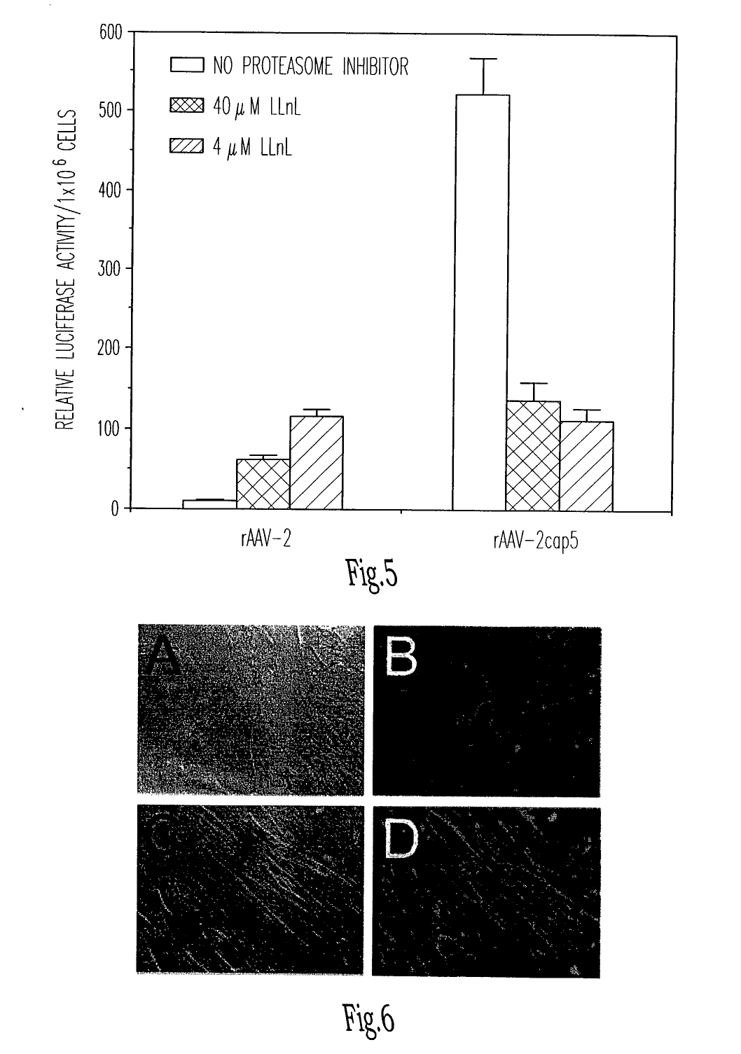

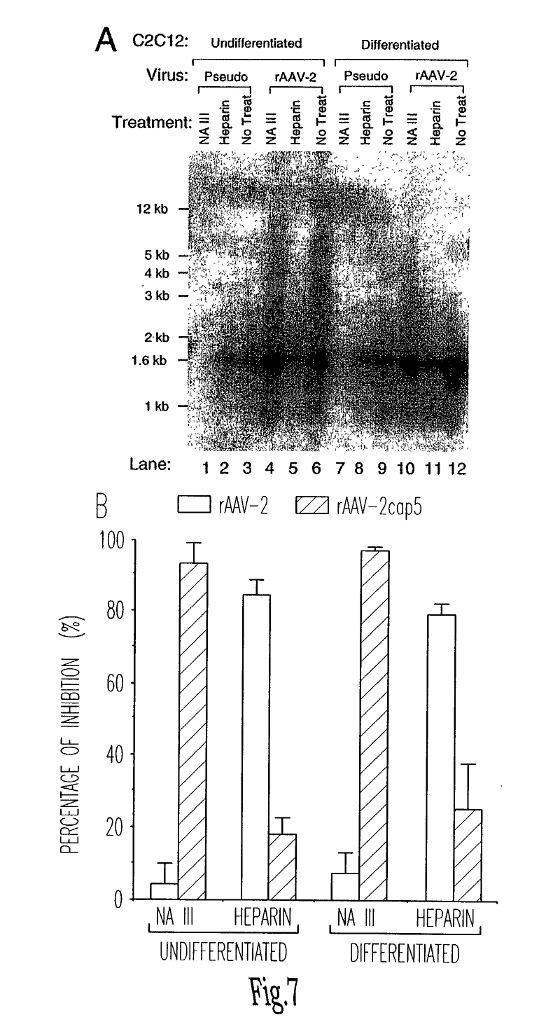

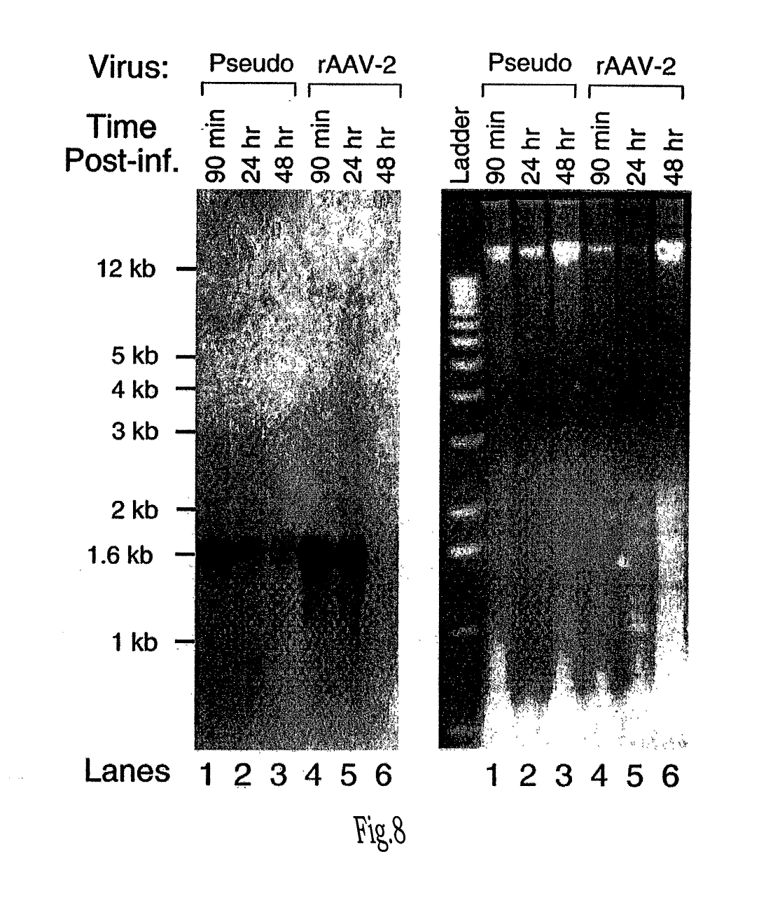

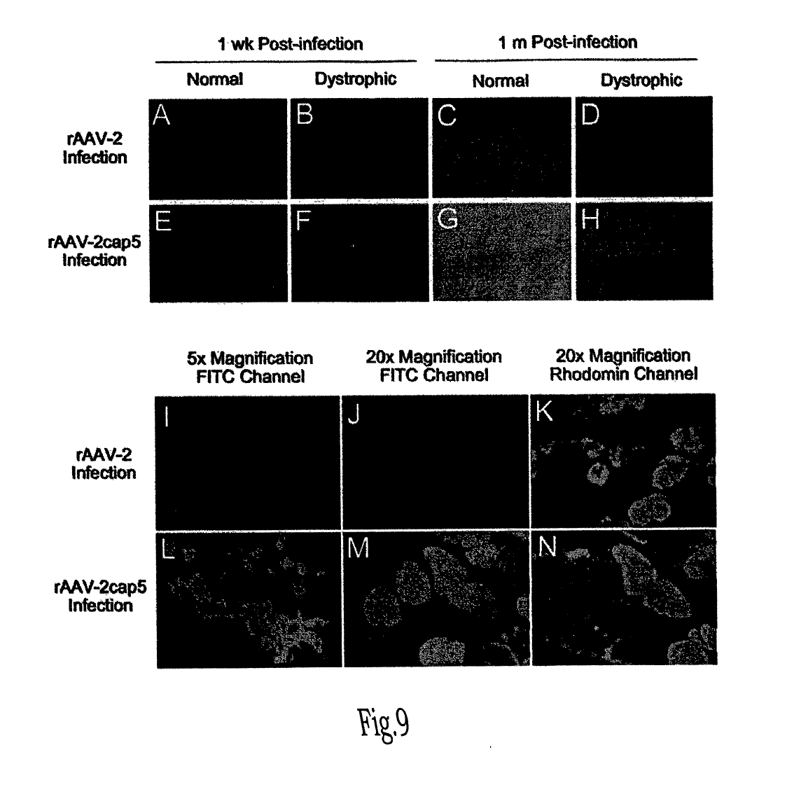

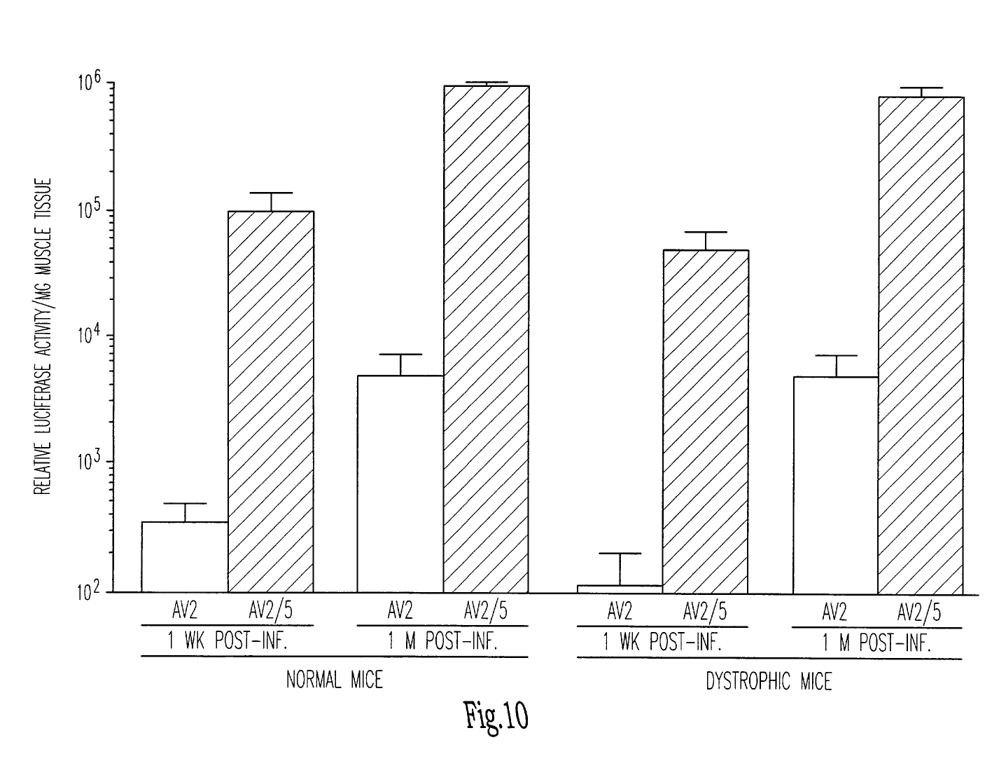

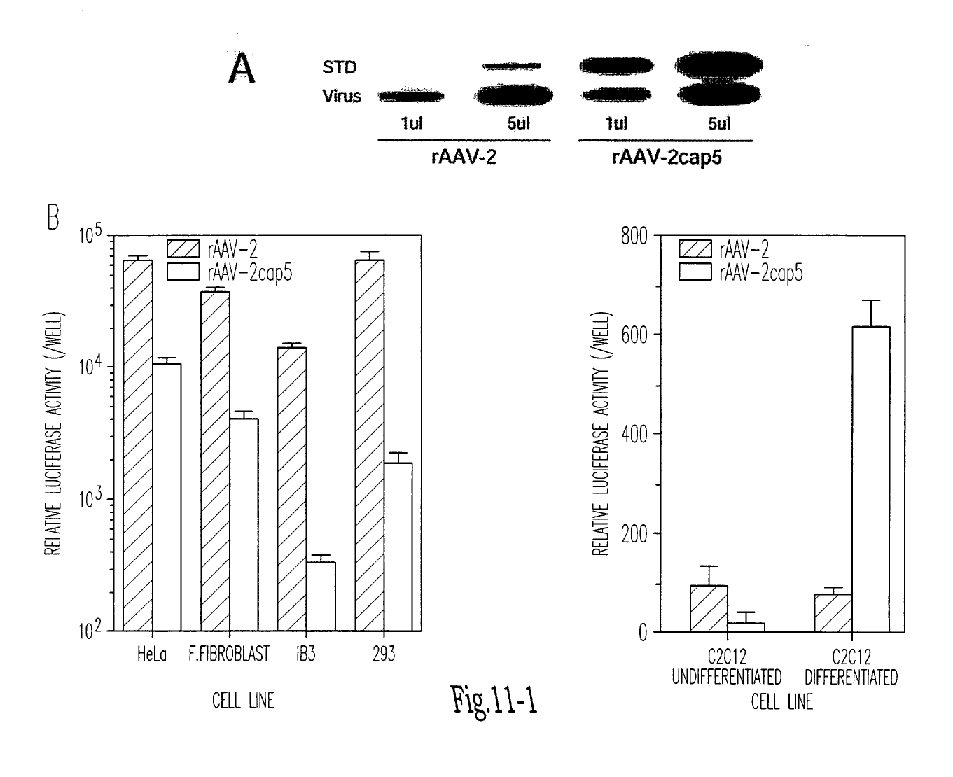

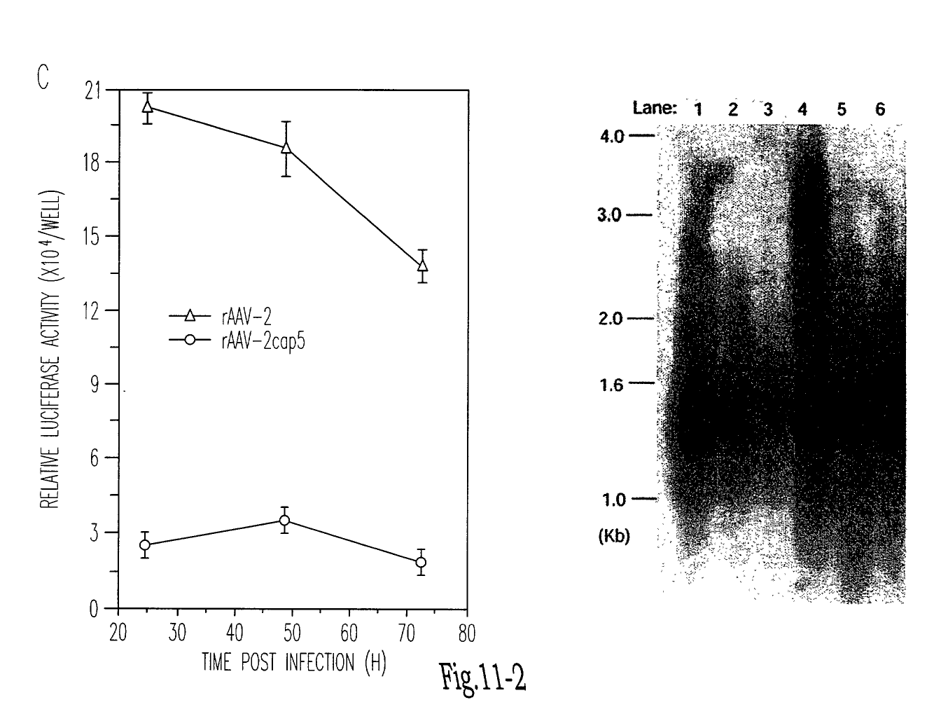

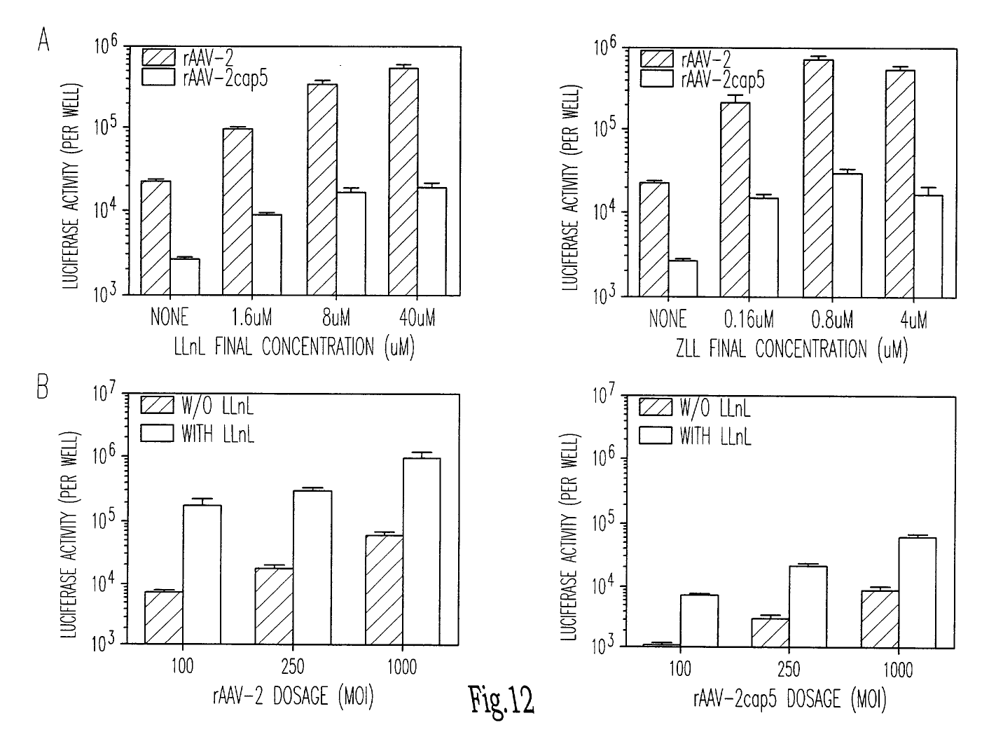

Encapsidation of rAAV-2 genome in AAV-5 capsid enhances transduction in differentiated, but not undifferentiated C2C12 cells. C2C12 cells are myoblast cells derived from the C3H strain of mice which can differentiate into contractile myotubes and produce muscle specific proteins. In undifferentiated C2C12 cells, no significant difference in transgene expression was observed with CMV driving EGFP vectors when the same numbers of DNA particles of rAAV2 or rAAV2cap5 were used for infection (FIG. 2). However, when differentiated C2C12 cells were infected under identical conditions, a dramatic increase in EGFP expression was observed in rAAV2cap5 infected cells but not in rAAV-2 infected cells (FIG. 2). Despite the apparent increase in transgene expression, quantifying the percentage of EGFP positive cells yielded little quantitative information on the average increase in transgene expression on a per cell basis.

To further characterize the time course of rAAV transduction and exclude promoter and/or transgene related artifacts, the study was repeated with vectors containing the RSV promoter driving luciferase. The use of the luciferase reporter gene also permitted a more sensitive and quantitative analysis. As shown in FIG. 3, low-level transduction was observed in undifferentiated myoblasts for both rAAV-2 and rAAV-2cap5 viruses. Consistent with findings using CMV-EGFP vectors, rAAV-2 mediated luciferase expression dropped an order of magnitude in differentiated C2C12 cells. In contrast, transgene expression from the rAAV-2cap5 virus was significantly enhanced in well-differentiated myotubes, with a greater than 500-fold increase in luciferase activity in comparison to undifferentiated cells at 72 hours post-infection (FIG. 3B). These findings suggested that pseudotyped rAAV-2cap5 virus might prove to be a more efficacious vector for gene delivery to post-mitotic myofibers in vivo.

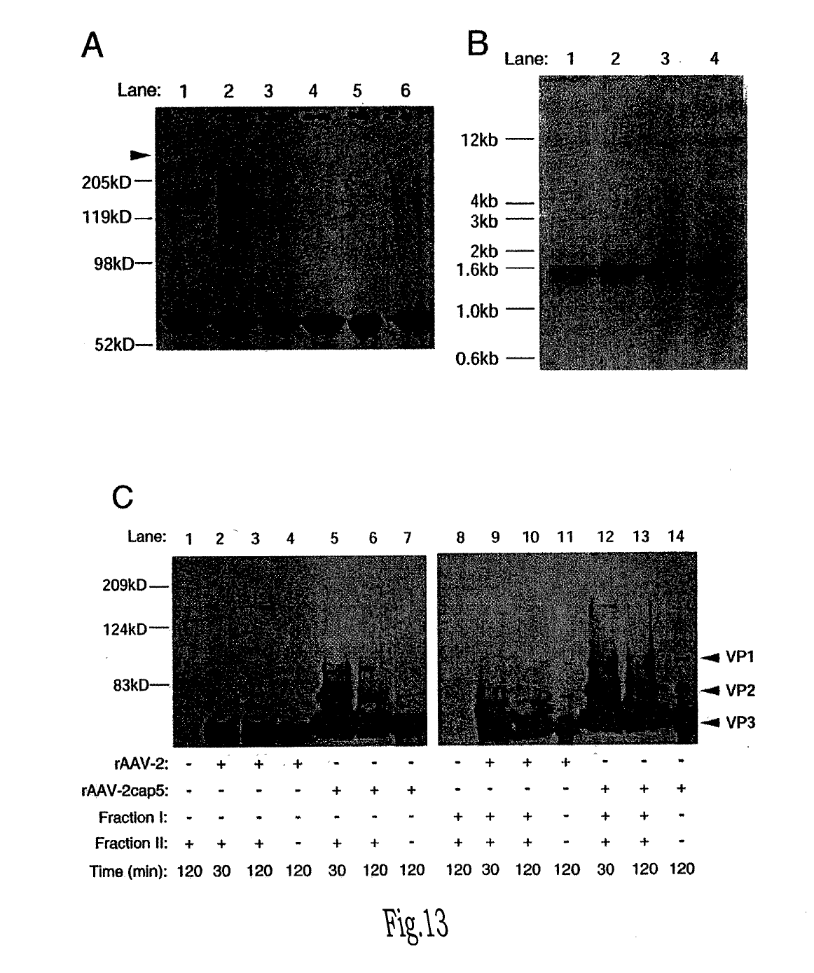

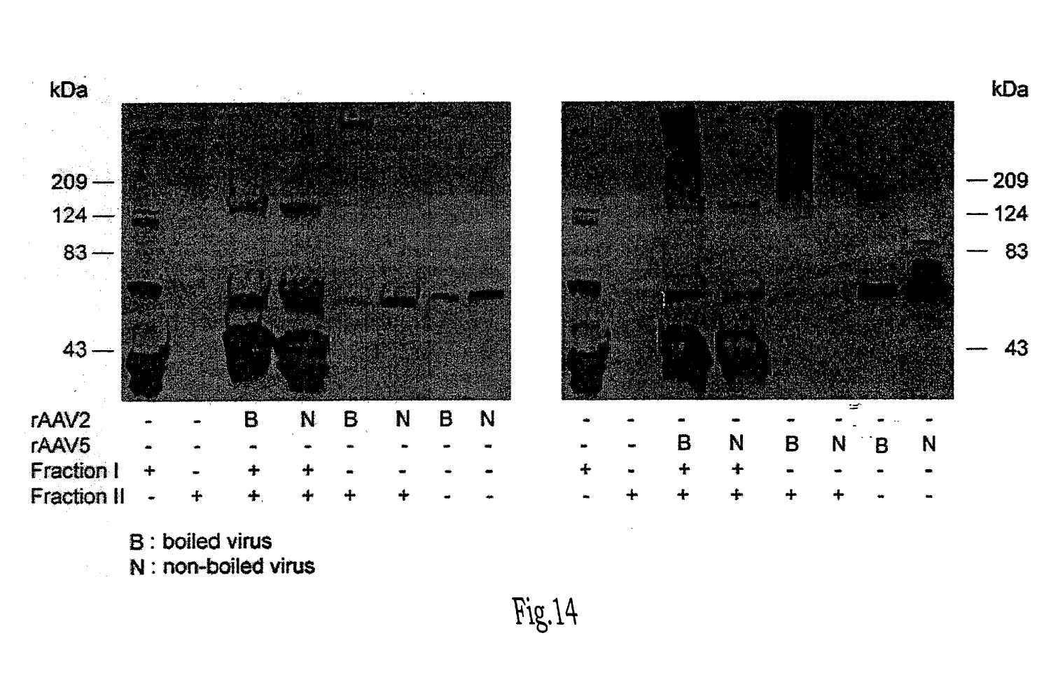

Differences in viral binding cannot explain the discordance in C2C 12 cell transduction with rAAV-2 and rAAV-2cap5 virus. Next, it was determined whether the different transduction profiles seen in differentiated C2C 12 cells were due to differences in viral binding, as might be anticipated by altered capsid structure. Previous studies have also suggested that factors affecting viral endocytosis also influence transgene expression from rAAV vectors (Duan et al., 1999; and Walters et al., 2000). To compare the viral binding efficiency, C2C12 cells (undifferentiated or differentiated) were incubated with rAAV-2 or rAAV-2cap5 virus at 4° C. for 60 minutes. Low molecular weight Hirt DNA was harvested from infected cells after PBS washing or trypsinization to remove extracellular bound virus. The overall viral binding to the cell surface was determined by Southern blotting of Hirt DNA (FIG. 4). Surprisingly, AAV-2 capsid, which provided poor transduction, mediated higher binding efficiency in both undifferentiated and differentiated C2C12 cells than the AAV-5 capsid (FIG. 4, lanes 6 and 12). Furthermore, surface bound rAAV-2 was easily removed by trypsin (FIG. 4, lanes 5 and 11). In striking contrast, irrespective of the cellular differentiation state, lower levels of the rAAV-2cap5 pseudotyped virus bound to the cell surface when compared to rAAV-2 under identical infection conditions. This data suggested that differences in endocytic mechanisms and/or intracellular processing, but not viral binding, must be responsible for the higher level of transduction seen with the pseudotyped virus.

To further dissect potential differences in intracellular processing between rAAV-2 and rAAV-2cap5, their transduction profile was compared following treatment with proteasome inhibitors. Tripeptide proteasome inhibitors have recently been shown to enhance persistent rAAV-2 transduction in polarized airway cells. This induction involves alterations in several aspects of viral endocytosis such as viral ubiquitination, endosomal processing and nuclear trafficking (Duan et al., 2000b). Therefore, response to proteasome inhibitors may indirectly reflect the molecular mechanisms by which AAV is processed through the endosomal compartment. Fully differentiated C2C 12 cells were infected with either rAAV-2 or rAAV-2cap5 at an moi of 600 particles/cell (FIG. 5). In the presence of either 40 μM LLnL or 4 μM Z-LLL, rAAV-2 transduction was increased 6 or 10-fold, respectively. Interestingly, application of LLnL or ZLLL resulted in a significant decrease in transgene expression in rAAV-2cap5 infected cells. This data strongly suggested that rAAV-2 and rAAV-2cap5 follow distinct intracellular pathways following endocytosis in differentiated C2C12 cells.

Southern blot analysis also revealed another interesting aspect of AAV-5 capsid binding. Trypsinization was initially used to confirm that the viral particles were not internalized during the 4° C. incubation (Duan et al., 1999; and Duan et al., 2000b). Two assumptions were made in this study. First, the plasma membrane is inert and lacks active endocytosis at 4° C. Second, stringent trypsinization (0.5% trypsin) should to remove all surface bound viral particles. This was indeed the case for rAAV-2 virus in many different cell types such as HeLa cells (Duan et al., 1999), primary cultured human airway epithelial cells (Duan et al., 2000b) and C2C12 cells (FIG. 4). Unexpectedly, a significant amount of trypsin-resistant viral DNA was detected in rAAV-2cap5 virus infected C2C12 cells. This data indicated that either a very efficient and/or fast internalization of AAV-5 capsid occurred, or that the interaction between the AAV-5 capsid and its receptor has a very high affinity and/or is relatively trypsin insensitive.

Increased transduction of rAAV-2cap5 pseudotyped virus in differentiated C2C12 cells correlates with increased viral binding. Information gained from viral binding studies at 4° C. also shed light on why differentiation of C2C12 cells leads to significant increases in transduction with rAAV-2cap5 virus. Consistent with increased transgene expression, an 8-fold increase in viral binding was observed for rAAV-2cap5 virus in differentiated cells as compared to undifferentiated cells (compare lanes 9 and 3 in FIG. 4). However, the magnitude of increased binding was approximately two orders of magnitude lower than the increase in transgene expression in differentiated cells (FIG. 3). These findings also suggested that enhanced viral binding of AAV-5 capsids cannot completely explain the increased transduction efficiency seen in differentiated myotubes.

Recently, 2,3-linked sialic acid was identified as a cellular receptor for rAAV-5 or is a necessary component of its receptor complex (Walters et al., 2000). Maackia amurensis lectin II (MAL II) preferentially binds to alpha-2,3-linked sialic acid and hence can be used to assess the abundance of this sialic acid form. To further characterize the enhanced binding of rAAV-2cap5 pseudotyped virus in differentiated C2C12 cells, the MAL II binding pattern in both undifferentiated and differentiated cells was examined. Consistent with the viral binding profile, cell surface expression of alpha-2, 3 linked sialic acid was significantly upregulated in differentiated cells as indicated by enhanced MAL II binding (FIG. 6).

To further analyze the interaction between sialic acid and the AAV-5 capsid protein, C2C12 cells were pre-treated with Type III NA sialidase. As was shown in FIG. 6, sialidase treatment completely abolished the AAV-5 capsid binding to C2C 12 cells (FIG. 7, lanes 1 and 7). However, identical treatment had only minimal effects on AAV-2 capsid binding in these cells (FIG. 7, lanes 4 and 10). As a control, the effect of free heparin on viral binding was also evaluated. Heparan sulfate proteoglycan (HSPG) has been reported as the primary attachment receptor for AAV-2 virus (Summerford et al., 1998). HSPG is also associated with the initial binding of many other viruses including herpes simplex virus and human immunodeficiency virus (Duan et al., 1999). Consistent with other reports, pre-incubation with free heparin dramatically decreased AAV-2 capsid binding in C2C12 cells.

Serotype specific capsid entry pathways effect the stability of viral genomes following infection. As discussed above, differences in the intracellular processing of virus following entry through distinct capsid receptors appears to be a determining factor which could explain the diverse transduction profile of rAAV-2 and rAAV-2cap5 pseudotyped virus in fully differentiated C2C12 cells. To further characterize this process, the kinetics of viral genome persistence with these two recombinant vectors was analyzed (FIG. 8). Important to this analysis is the fact that the two recombinant viruses differ by only their capsid structures and contain identical viral genomes. Differentiated C2C12 cells were infected at the same particle moi with either rAAV-2 and rAAV-2cap5 at 4° C. for 90 minutes. Hirt DNA was prepared either immediately following infection at 4° C. or at 24 and 48 hours following a shift to 37° C. Consistent with findings shown in FIG. 4 and FIG. 7, rAAV-2 virus attached to differentiated C2C12 cells more efficiently during the 90 minute incubation at 4° C. However, by 48 hours post-infection at 37° C., the intracellular level of single stranded viral genomes delivered by AAV-2 capsid dropped to almost undetectable level. Interestingly, the viral genomes introduced by AAV-5 capsid were significantly more stable. Since the only difference between pseudotype virus and the rAAV-2 was the viral capsid, it was hypothesized that different pathways for processing internalized AAV-2 and AAV-5 viral capsid encoded genomes affect viral genome persistence. However, it should also be stressed that the 1.6 kb single stranded viral genome is not directly responsible for transgene expression. Nonetheless, these genomes are precursors for genome conversion to a transgene expressible form and hence the stability of single stranded DNA viral genomes will likely affect the extent to which virus can ultimately express an encoded transgene.

AAV-5 capsids mediate increased transduction of normal and dystrophic muscle. To further expand the in vitro findings, the transduction efficiency of both pseudotyped rAAV-2cap5 and native rAAV-2 in mouse skeletal muscle was examined. Two sets of experiments were carried out with viruses harboring either a CMV-EGFP or an RSV-luciferase expression cassette. Transgene expression was evaluated at 1 week and 1 month after infection. Consistent with results in Duan et al. (1998), rAAV-2 mediated EGFP expression was barely detectable at 1 week post-infection in normal muscle (FIG. 9A). In sharp contrast, at 1 week post-infection, a significantly higher level of EGFP expression was detected in normal muscle infected with rAAV-2cap5 virus (FIG. 9E). Evaluation of the transgene expression 1 month after infection also demonstrated a much higher EGFP expression in normal muscle infected with rAAV-2cap5 as compared to rAAV-2 (FIGS. 9G and 9C).

A previous report has suggested that rAAV-2 transduction in dystrophic muscle may be significantly decreased due to the disease process (Cordier et al., 2001) and so pseudotyped rAAV-2cap5 virus might impart some level of increased transduction in diseased mdx skeletal muscle. As seen in normal muscle, rAAV-2cap5 infection afforded significantly higher levels of transduction in mdx muscles (FIGS. 9F and 9H) when compared to native rAAV-2 virus infection (FIGS. 9B and 9D). However, the level of rAAV mediated EGFP expression was significantly reduced in mdx mice infected with either rAAV-2cap5 or rAAV-2 virus as compared to normal control littermates (FIGS. 9A-H).

EGFP expression in dystrophic muscle was also examined at 6 months post-infection. Consistent with the 1 week and 1 month findings, prominent EGFP expression was found only in rAAV-2cap5 infected muscle samples (FIGS. 9I-N). Very few EGFP positive myofibers were detected in rAAV-2 infected muscles. Furthermore, the intensity of EGFP expression in each individual myofiber was also much lower in the rAAV-2 infection group. Of interest, Evans blue positive, damaged myofibers appeared to be transduced at an equal efficiency to non-damaged Evans blue negative myofibers by rAAV-2cap5 (FIGS. 9J, 9K, 9M and 9N).

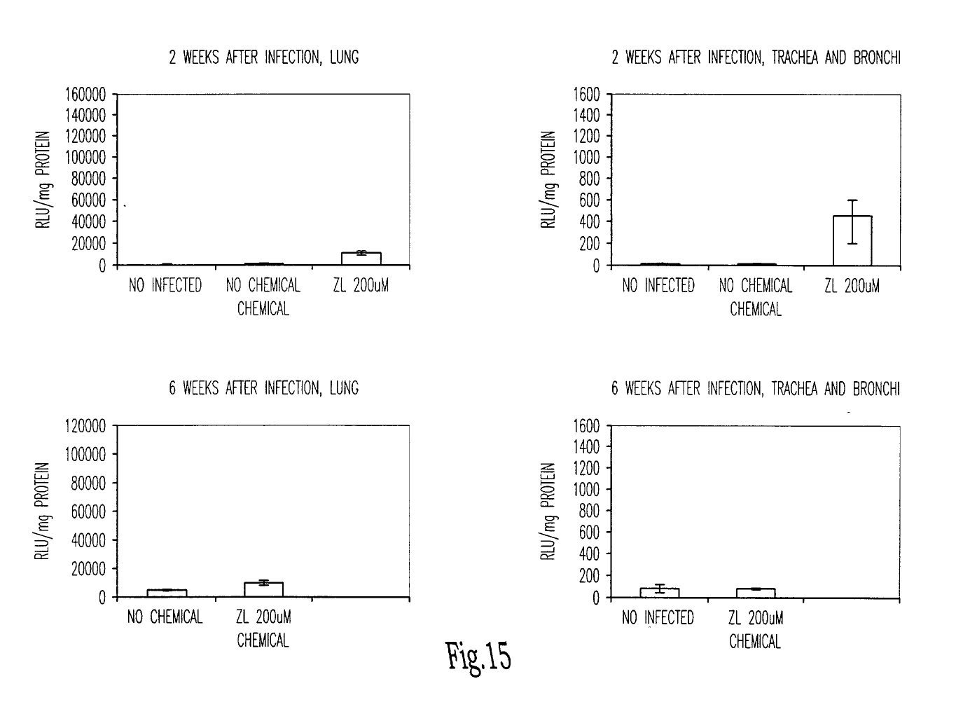

In an effort to obtain a more quantitative understanding of the transduction profiles in normal and dystrophic muscles, viruses carrying the more sensitive RSV-luciferase expression cassette were used. As demonstrated in FIG. 10, rAAV-2cap5 virus infection resulted in a greater than 200-fold enhancement in luciferase expression at 1 week and 1 month post-infection when compared to native rAAV-2 virus. Surprisingly, a similar profile of enhancement was achieved in both normal and dystrophic muscle. Several aspects of the reporter gene and/or the methods used for detection could have potentially influenced the discordance in dystrophic muscle expression of EGFP and/or luciferase reporters. These include the half-life, immunogenicity of the transgene products in the setting of diseased myofibers, and the sensitivity of the transgene expression assays (minimal threshold and maximal saturating levels for detection). Luciferase is very sensitive to protease degradation, and in transfected mammalian cells, its half-life is about 3 hours (Thompson et al., 1993). In contrast, GFP is extremely stable and has a longer half-life (Ward et al., 1982). Therefore, it was unlikely that disease induced alterations in the degradation of the reporter proteins can explain these observations. A previous study has suggested that immunoreactivity of a transgene encoded protein is a critical determinant for the stability of transgene expression in immuno-competent mice (Tripathy et al., 1996). Hence, it is plausible that in the setting of Duchenne's muscular dystrophy, EGFP may be more immunogenic than luciferase. Despite these potential issues with the immunogenicity of EGFP and luciferase, the data clearly demonstrated that rAAV-2cap5 pseudotyped virus was much more effective (>200-fold) in transducing both normal and mdx skeletal muscle. Given the identity of the viral genomes in both native rAAV-2 and pseudotyped rAAV-2cap5 virus, these findings implicate AAV type 5 capsid interactions with myofibers as the sole determinant for increased transduction.

Discussion

In this study, the transduction efficiency of identical rAAV-2 genomes delivered by two different viral capsids was examined. This capsid modification strategy has been extensively used by many researchers to either direct targeted expression or improve the transduction efficiency for certain cells which are less transducible with rAAV-2 (Girod et al., 1999; and Wu et al., 2000). The rational for this study was based on the recent findings that rAAV-5 can significantly enhance rAAV mediated gene transfer in certain cell types (Davidson et al., 2000; and Zabner et al., 2000). Since the homology for both viral ITR and capsid proteins is only about 60% between AAV-2 and AAV-5, it is conceivable that either the viral genome or the capsid structure could be responsible for the improved transduction efficiency with rAAV-5. To better understand the functional contribution of the viral capsid alone, a hybrid viral system was utilized in which rAAV-2 genomes were packaged in AAV-5 capsids. This pseudotyped virus should comparatively eliminate any contributions of the viral genome on transduction efficiency. Both in vitro studies in differentiated cells and in vivo data in mouse skeletal muscle indicated that pseudotyped virus was significantly more efficient in mediating transgene expression than native rAAV-2 virus.

One unexpected finding was that the transduction efficiency of rAAV was significantly affected by the cellular state of differentiation in C2C12 cells. Furthermore, the influence of differentiation had opposite effects for the two serotypes of rAAV analyzed. In the case of rAAV-2 infection, differentiation of C2C 12 cells decreased viral transduction by 10-fold. In contrast, differentiation increased transgene expression with pseudotyped rAAV-2cap5 virus by more than 500-fold. The differentiation of the myoblasts into contractile myotubes involves the coordinated expression of many cellular factors. When growth factors are deprived (as is the case for inducing differentiation of C2C12 cells), the proliferating myocytes enter a terminal differentiation stage and start to express various differentiation factors (such as myogenin, p21/WAF1) and contractile proteins (such as myosin and troponin) (Walsh et al., 1967). It is currently not clear what factors are directly linked to the enhanced transduction of differentiated cells by pseudotyped virus. However, the data described herein do suggest that the differentiation-associated changes in cell surface lectin expression contribute to the increased viral binding of AAV-5 capsids to myotubes following pseudotyped virus infection. Nevertheless, binding of rAAV to the cell surface appeared not to be the primary determinant of differences in the transduction efficiency between rAAV-2 and rAAV-2cap5 viruses. The overall attachment of rAAV-2cap5 to muscle cells was weaker than that for rAAV-2. Furthermore, unlike rAAV-2, transduction of differentiated myotubes with pseudotyped rAAV-2cap5 virus was negatively regulated by proteasome inhibitors. Thus, differentiation induced changes in the intracellular characteristics of myotubes might be a more important factor contributing the higher level of transduction with rAAV-2cap5. For example, muscle differentiation might enhance intracellular processing and/or uncoating of incoming pseudotyped virions. Alternatively, cellular differentiation could also adversely effect the intracellular movement of AAV-2 capsid packaged virions and lead to lower transduction. Furthermore, differentiation might alter the rate of internalization of AAV-5 but not AAV-2 receptors at the membrane.

The results from in vivo analyses comparing rAAV-2 to rAAV-2cap5 virus were also quite interesting. Several previous reports have suggested that rAAV-2 efficiently transduces dystrophic skeletal muscle and produces high levels of therapeutic proteins, including different sarcoglycans and micro-dystrophin (Cordier et al., 2000; Greelish et al., 1999; Li et al., 1999; and Wang et al., 2000). However, recent studies also suggest that rAAV-2 mediated transgene expression is significantly reduced in dystrophic muscle if the transgene is driven by a ubiquitous viral promoter (Cordier et al., 2001). This has been attributed to ectopic transgene expression in antigen presenting cells and subsequent immune clearance of the transgene expressing cells. The studies described herein evaluating rAAV mediated RSV-luciferase gene delivery demonstrated little difference in gene expression between normal and mdx muscles. However, results evaluating rAAV mediated EGFP expression in mdx mice were quite different. That is, despite a decreased EGFP expression in rAAV-2 infected mdx muscle, high-level transduction was observed following infection with rAAV-2cap5 pseudotyped virus (FIGS. 8I-N). Although rAAV-2cap5 mediated EGFP gene expression was lower in mdx than in normal muscles, compared with rAAV-2, there appeared to be a lower degree of disease associated effects on transgene expression with rAAV-2cap5 virus. Since different capsid structures determine the dissimilar cellular tropisms of AAV-2 and AAV-5 (Davidson et al., 2000 and Zabner et al., 2000), differences in disease associated effects on rAAV-2 and rAAV-2cap5 EGFP expression might be explained by a decreased susceptibility of dendritic cells to AAV-5 infection.

Recent studies have suggested that rAAV-2 is capable of circumventing the maturation-dependent barrier of muscle gene transfer by other viruses including adenovirus, retrovirus and herpes virus (Pruchnic et al., 2000). Since myofiber maturation and myoblast differentiation represent distinct biological processes, it remains to be determined whether AAV-5 capsid can provide additional benefits in overcoming this barrier. It has also been suggested that rAAV-2 preferentially transduces Type I slow myofiber, and this propensity might be associated with the overexpression of rAAV-2 receptor heparan sulfate proteoglycan. Further examination of potential myofiber subtype preferences for AAV-5 capsid infection may uncover further mechanistic insights into how AAV-5 pseudotyping increases transduction in differentiated muscle.

In summary, these studies shed light on biological differences between AAV-2 and AAV-5 capsids and their effect on cell-vector interactions in muscle cells. Differences in the biology of viral infectious processes between these two vectors significantly affect their efficiency to deliver transgenes into differentiated myofibers. Interestingly, skeletal muscle has been traditionally thought to lack many of the barriers to rAAV-2 infection seen in other tissues such as the airway. However, comparative studies between rAAV-2 and rAAV-2cap5 suggest that muscle may also have similar barriers to rAAV-2 infection involving endocytosis and/or intracellular processing that limit its full utility as a gene therapy vector. In this context, a principle lesson from these studies is that the efficiency of viral binding does not always directly correlate with transduction efficiency. This is not entirely surprising given the reported influences of co-receptor(s) in endocytosis of rAAV vectors. Studies evaluating phenotypic differences induced by myoblast differentiation may begin to shed more light on the cellular factors controlling the efficiency of AAV endocytosis and/or intracellular processing.

### EXAMPLE 2

### Both Adeno-Associated Virus Type 2 and 5 are Substrates for Ubiquitination Which Affects Transduction Efficiency in Several Cell Lines

The effect of proteosome inhibitors on AAV-2 and AAV-5 transduction was compared using transgene expression. The AAV-5 ITR is only 58% homologous with the AAV-2 ITR (Chlorinin et al., 1999) and it is possible that mechanisms for viral trafficking and DNA strand conversion could be different between these two types of recombinant AAV. To exclude possible effects of the AAV ITRs on transgene expression, an identical AAV-2 transgene construct was packaged into either the AAV-2 or AAV-5 capsids. Transgene expression assays for the native rAAV-2 virus and the AAV-5 pseudotyped virus facilitated direct evaluation and comparison of transduction efficiencies of these two different serotypes under the same infection conditions.

Materials and Methods

Cloning of the Helper Plasmids for Pseudotyping. Isolated wild type AAV-5 viral DNA was annealed by heating at 95° C. for 5 minutes, followed by overnight, slow cooling to 60° C. A PCR approach permitted cloning of the full length AAV-5 coding region by reassembling two PCR products with a unique restriction enzyme site. The primer set for AAV-5 Rep were: forward:

5′-gctctagaGATGTAATGCTTATTGTCACGCGA-3′ (SEQ ID NO:1); reverse:

5′-cccaagcttGATTGGGTTTTGGTTTCGGTGGGC-3′ (SEQ ID NO:2). For AAV-5 Cap, the primers were: forward:

5′tgcactgcagGCGAGTAGTCATGTCTTTTGTT GATCACCC-3′(SEQ ID NO:3) reverse: 5′-cccaagcttcgtctagaGACCACAAGAGGC AGTATTTTACTGAC-3′ (SEQ ID NO:4). Homologous sequences to AAV gene components are presented in upper case bases and lower case bases represent overhangs to cloning restriction sites (underlined).

The 2.1 kb AAV-5 Rep and 2.3 kb Cap coding regions were amplified separately and each fragment was subcloned into pBluescript SKII. With the unique BclI site in the overlapped region of each fragment, the two AAV-5 fragments were ligated to generate a 4.3 kb AAV-5 genome with no ITR structure at either end. The helper plasmid for AAV-5 packaging (pAV5-Trans) was generated by replacing the AAV-2 sequence in the AAV-2 packaging helper plasmid (pAAV-2/Ad) (Samulski et al., 1989) with the 4.3 kb full-length AAV-5 coding fragment. A second helper plasmid with only the AAV-2 Rep sequence (pAV2-Rep) was generated by deleting the 1.1 kb ApaI fragment in the AAV-2 Cap coding region of pAAV-2/Ad. To confirm that no AAV-2 capsids were generated, western blotting of Ad5.CMVlacZ infected 293 cell lysate transfected with pAv2Rep was performed.

Generation of rAAV Stocks. Stocks of the native rAAV-2 virus (rAAV-2RSVluc) and the rAAV-5 pseudotyped virus (rAAV-2-cap5RSVluc) were generated with plasmid pcisAV2RSVluc, described in Duan et al. (2001a). This rAAV-2 proviral plasmid encodes an RSV LTR promoter-driving the luciferase gene flanked with two AAV-2 ITRs from pSub2O1. A routine CaPO₄ co-transfection protocol was used to produce rAAV from Ad5.CMVlacZ coinfected 293 cells. To produce native rAAV-2 virus, the co-transfection protocol included the proviral plasmid pcisAV2RSVluc with pAAV-2/Ad at a ratio of 1:3. rAAV-2-cap5 pseudotyped virus was generated by transfecting the same rAAV-2 construct, pcisAV2RSVluc, into adenovirus infected 293 cells together with pAV2-Rep and pTrans-AV5 at a ratio of 1:1:3. Cells were harvested 40 hours after transfection and virus particles were released by freeze thawing, DNase I digestion and deoxycholate treatment. Both viral stocks were purified using the same CsCl₂ ultracentrifugation procedure.

Following 3 rounds of CsCl₂ banding, 1.36 to about 1.42 g/cm³ fractions were collected. To inactivate any possible remaining adenovirus contamination, the AAV fractions were heated at 60° C. for one hour. After dialysis against Hepes buffered saline at 4° C. for 2 days to remove the CsCl₂, the viral stocks were quantified by slot blot and transgene expression was tested in cultured cells. Contamination with wild type AAV-2 was determined and found to be less than one functional particle per 1×10¹⁰ rAAV-2 particles. Wild type AAV-2/5 hybrid contamination was evaluated by nested PCR for the Rep and Cap genes. Less than one particle of the wild type hybrid virus was detected in 1×10¹⁰ pseudotyped viral particles (see Example 1). Contamination with helper adenovirus, Ad5.CMVlacZ, was evaluated by histochemical staining for 13-galactosidase activity. Typically, helper virus contamination is less than 1 in 1010 DNA particles.

Transduction of Cells in vitro. HeLa, 293, and IB3 cells and primary fetal fibroblasts were cultured as monolayers in Dulbecco's Modified Eagle Medium (DMEM), supplemented with 10% fetal bovine serum and penicillin (100 U/ml)-streptomycin (100 μg/ml), and maintained in a 37° C. incubator at 5% CO₂. Undifferentiated C2C12 muscle cell line was similarly cultured in the condition, however differentiation was induced by feeding the cells with horse serum rather than FBS. Typically well differentiated cultures of C2C12 cells developed by 5-7 days following the addition of 10% horse serum at which time they were used for experiments (Example 1; Yaffe et al., 1977). All other cell lines were seeded in 6-well (1×10⁶/well) or 12-well (5×10⁵/well) plates and allowed to adhere for 18 hours. One hour prior to infection, cells were re-fed with fresh medium with or without proteosome inhibitors. The tripeptidyl aldehyde proteosome inhibitor N-acetyl-L-Lueucyl-L-Luceucyl-norluecine (LLnL, or MG110) was purchased from Boston Biochem (Boston, Mass.) and Carbobenzoxy-L-Leucyl-L-Luecyl-L-leucinal (ZLL, also referred to as Z-LLL or MG132) was from Calbiochem-Novabiochen (La Jolla, Calif.). These inhibitors were dissolved in DMSO as a 1000× stock solution with LLnL at 40 mM and ZLL at 4 mM and stored at −20° C. Virus infection was performed in serum-free DMEM and an equal amount of DMEM-20% FBS was added at 2 hours post-infection to bring the final serum level to 10%. In the case of infections with proteosome inhibitor, typical final concentrations were 40 μM LLnL and 4 μM ZLL. The chemicals were diluted in the culture medium and treatment was performed with a 1 hour pre-infection incubation and continued presence in the media during the 24 hour infection.

Transduction Analysis. For analysis of transgene expression, luciferase activity in infected cells was measured with an assay kit from Promega 24 hours after infection. Cells were lysed with 200 μl lysis buffer in each well of the 12-well plates. Cell membranes and debris were pelleted by micro-centrifugation at 10,000×g for 1 minute. The supernatant was reacted with the luciferase substrate according to the procedure described in the assay manual. A luminometer (TD-20/20, Turner Designs Instrument, Sunnyvale, Calif.) determined luciferase activity at a sensitivity of 70%. For the viral DNA assay, low molecular weight DNA was extracted according to the Hirt procedure with modifications as previously described in Yan et al. (2000). The Hirt DNA from 2×10⁶ infected cells was dissolved in 50 μl TE and one-half was resolved on a 1% agarose gel. Southern-blots of the viral DNA were hybridized with a luciferase fragment probe labeled with a-P³²-dCTP by random priming.

Immunoprecipitation of Ubiquitinated AAV Capsid. Detection of AAV ubiquitination in Hela cells treated with proteosome inhibitor was performed as described in Duan et al. (2000) with modifications. 2×10⁶ Hela cells were infected with 2×10⁹ DNA particles of rAAV-2RSVluc or rAAV-2cap5RSVluc, in serum-free DMEM. Infections were performed in parallel, with or without the presence of 40 μM LLnL. Four hours after infection, cells were lysed in 0.8 ml RIPA buffer. Cell lysates were pre-cleared with 10 μl ProteinG PLUS-Agarose (Santa Cruz Biotech) and were then incubated with 10 μl of mouse Anti-VP 1-3 monoclonal antibody (Clone B1, American Research Products) at 4° C. for 1 hour, followed by the addition of 30 μl Protein G PLUS-Agarose. After overnight incubation at 4° C., the beads were washed four times with 1 ml ice-cold RIPA buffer and resolved on 10% SDS-PAGE. After transfer to a nitrocellulose filter, the blot was probed with a 1:200 dilution of anti-Ubiquitin monoclonal antibody (Clone P4D1, Santa Cruz Biotech), followed by 1:2000 horseradish peroxidase conjugated second antibody. After the final washings, the ubiquitinated viral protein was visualized with the ECL system (Amersham Pharmacia).

In vitro Ubiquitination of AAV Particles. All the reagents used for the in vitro ubiquitination assay were purchased from Boston Biochem, Inc. (Boston, Mass.). The ubiquitin-protein conjugation kit (Cat# K960) consists of ATP containing energy buffer, ubiquitin substrate solution and the purified conjugation enzymes (E1, E2s and E3s) from HeLa cell cytoplasm extract Fraction II. Additionally, since not all potential E2s and E3s are present in this extract, Fraction I extract (Cat# F-375) distinguished from Fraction II extract by their anion exchange binding characteristics, can be supplemented to ubiquitination reaction. HeLa Cell Fraction I provides additional E2s and E3s, that are not represented in Fraction II extract (Hershko et al., 1983). Fraction II does not contain 20S and 26S proteosomes or other protein degradation activity, but contains ubiquitin C terminal hydrodases (UCHs). To improve the yield of the ubiquitinated protein product, ubiquitin aldehyde (Ub-H, Cat # U-201) was used for the inhibition of UCHs activity (Melandri et al., 1996). Fraction I extracts do contain proteasome activity which must be inhibited by LLnL (200 μM) during the reaction. The ubiquitin conjugation to purified AAV virions was performed according to standard protocols provided by the supplier with modification. In brief, 25 μg of Fraction II enzyme conjugation components, 60 μg ubiquitin, and 2 μg ubiquitin aldehyde, 5 μl 10×energy buffer were mixed and brought to a final 50 μl reaction volume with 50 mM Hepes buffer, pH 7.6. The mixture was incubated at 37° C. for 5 minutes to allow for inhibition of the UCHs. The conjugation was initiated by addition of 1 μl virus solution, which contained 3×10⁸ particles of rAAV-2 or rAAV-2cap5 virus. After a 1 hour incubation at 37° C., the reaction was quenched by addition of EDTA (10 mM final), and concentrated to about 15 μl via a Speed-Vac. The sample was mixed with SDS-loading buffer and resolved on a 10% SDS-PAGE. The AAV viral protein was analyzed via western blotting with anti-AAV capsid monoclonal antibody B1. Ubiquitination was visualized by an increased apparent molecular weight of immunoreactive capsid protein. To test whether the ubiquitination of AAV particles required additional E2s and E3s enzymes not found in Fraction II, the same reaction conditions described above supplemented with 12.5 μg HeLa cell extract Fraction I and conjugation similarly evaluated by Western blot analysis. To inhibit the proteasome activity introduced with Fraction 1,200 μM LLnL (final concentration) was also added only when Fraction I was used.

Results

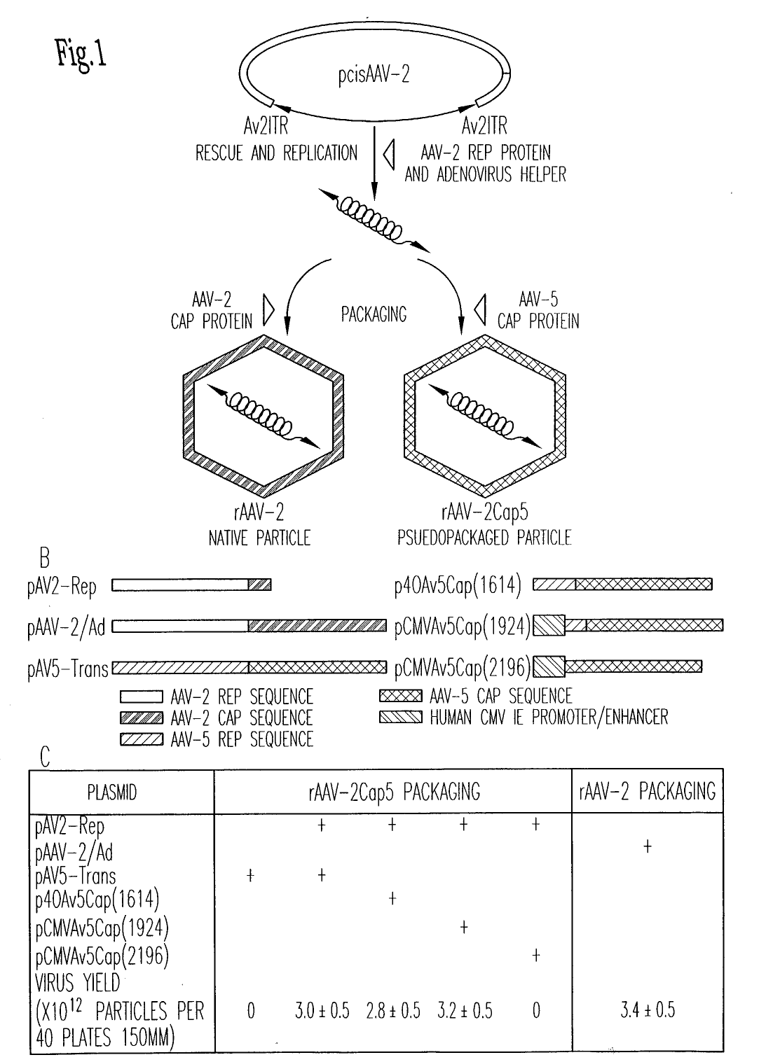

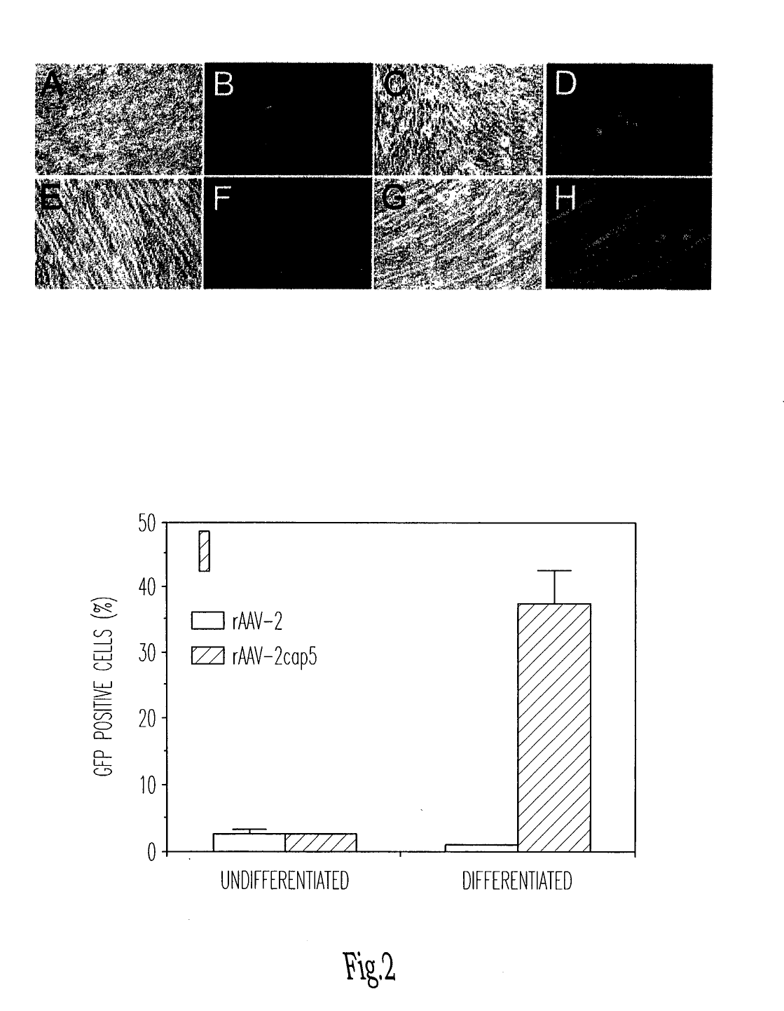

Pseudotyping the rAAV-2 genome with AAV-5 capsid proteins. Unlike other serotypes of AAV that have shown cross-complementation of ITRs and Rep genes, AAV-5 is more distinct. The 58% homology between the ITR of AAV-2 and AAV-5 and the low conservation of Rep protein binding and TRS recognition motifs suggests that the AAV-5 Rep and ITR will not complement with AAV-2. However, in the presence of the AAV-2 Rep proteins, rAAV-2 constructs can be pseudo-packaged by AAV-5 capsid to assemble infectious particles (Chlorini et al., 1999). As shown in FIG. 1A, the initial goal was to create a pseudo-AAV-5 virion packaged with a rAAV-2 genome encoding an RSV-driven luciferase reporter, in order to directly compare the efficiency of transduction with a native rAAV-2 virion. When the rAAV-2 proviral plasmid pcisAV2RSVluc was transfected together with a AAV-2 Rep protein expression plasmid (pAV2-Rep) in adenovirus infected 293 cells, the progeny viral DNA could be efficiently packaged into either an AAV-2 capsid or an AAV-5 capsid, depending on complementing capsid expression plasmid used. After purification of the viruses by isopycnic density gradient centrifugation, quantification by DNA slot bolt indicated that a similar packaging efficiency was obtained for rAAV-2 and rAAV-2cap5 viruses. The typical production yield was 3×1O12 particles/batch culture of forty 150 mm plates. No significant difference in efficiency was found for packaging pcisAV2RSVluc in rAAV-2RSVluc or rAAV-2cap5RSVluc (FIG. 1).

Since AAV-2 has been under development as gene transfer vector for a longer time, there is a greater understanding of the mechanisms for viral production than for AAV-5. For example, it is known that reduced AAV-2 Rep 68/78 protein expression results in a much higher yield of rAAV-2 virus (Li et al., 1997; and Xiao et al., 1998). To increase the pseudotyped virus packaging efficiency, the AAV-5 Rep gene coding region was deleted from the helper plasmid pAV5-trans. As shown in FIGS. 1B and 1C, disabling AAV-5 Rep protein expression resulted in no improvement in the yield of the pseudo-packaged rAAV-2cap5 virus. Similarly, substituting a strong, consistent heterologous promoter (the CMV immediate early promoter/enhancer) for the AAV-5 p40 sequence resulted in only a slight increase in yield. These results imply that the AAV-5 Rep proteins may act via an entirely different mechanism than the AAV-2 Rep proteins in transactivation of the p40 promoter driving AAV-5 Cap expression.

To confirm that the native and pseudotyped AAV-2 vectors were packaged as expected, the immunologic characteristics of the native rAAV-2RSVluc and the pseudotyped rAAV-2cap5RSVluc were evaluated. The mouse monoclonal antibody A20 (American Research Products), which only recognizes intact AAV-2 particle, did not demonstrate immunoreactivity to DNase-resistant particles of rAAV-2cap5 as assessed by either dot-blotting assay or immunoprecipitation assays followed by Southern blotting for viral DNA (data not shown). In contrast, a different monoclonal antibody termed B 1 reacted with both viruses with the same sensitivity on Western blots (data not shown). B1 is a commercially produced anti-AAV-2 antibody that recognizes VP1, VP2 and VP3. The B1 epitope is apparently located in a region with high homology between AAV-2 and AAV-5. When similar number of purified DNase resistant particles were evaluated by denaturing slot blot or Western blot, indistinguishable levels of immunoreactivity were seen against B 1 antibody. Furthermore, the ratio of VP 1,2, and 3 capsid proteins were also indistinguishable for both purified rAAV-2 and rAAV-2cap5.

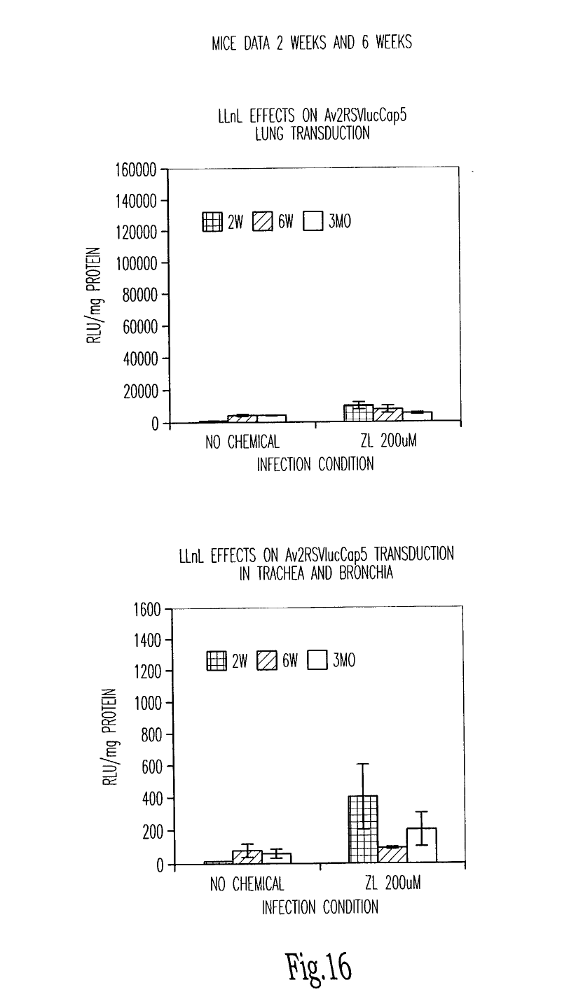

Encapsidation of rAAV-2 genomes in the AAV-5 capsid alters the efficiency of transgene expression in HeLa cells. Given the varying tropism for AAV-2 and AAV-5 in different cell lines, functional titering as a basis for comparison is problematic. To this end, equivalent titers of DNAse resistant physical particles were used as the basis for comparison as overall differences in the extent of baseline transduction were less of a concern. When equivalent numbers of physical particles of rAAV-2RSVluc or rAAV-2cap5RSVluc were used for infection (FIG. 11A), transgene expression was consistently 6-15 fold lower for rAAV-2cap5 virus in nearly all cell lines (HeLa cells, primary fetal fibroblasts, IB3 cells, 293 cells and undifferentiated C2C12 muscle cells) (FIG. 11B). This was with one exception where differentiated C2C12 cells gave an approximately 30-fold higher luciferase expression from rAAV-2cap5 in comparison to rAAV-2.

A possible explanation for the difference in transduction between the two viruses in these cell types might be the levels of their respective cell surface receptors. For AAV-2, heparin sulfate proteoglycan (HSP) is the primary receptor, and 2,3 linked sialic acid has been identified as the receptor for AAV-5. In support of this notion, induction of rAAV-2cap5 infection in differentiated C2C12 cells is in part due to increased 2,3 linked sialic acid at the membrane (see Example 1).

To further investigate whether receptor binding or endocytosis accounted for the observed differences in transduction efficiency, low molecular weight Hirt DNA was purified from each cell type infected in parallel with the two viruses and Southern blot analysis for intracellular viral DNA was performed. In fetal fibroblasts, IB3, and 293 cells, lower transduction for the pseudotyped virus appears to reflect lower uptake of the virus, since only a limited amount of viral DNA could be retrieved from these cell lines after rAAV-2cap5RSVluc infection (data not showed). However, in Hela cells, the amount of internalized viral DNA was similar for the two viruses, and thus the difference appears to be due to some aspect of intracellular processing. FIG. 11C demonstrates a kinetic analysis of the time course of transgene expression and uptake of viral DNA in Hela cells. The transgene expression level mediated by the native rAAV-2 was highest 24 hours after infection, and decreased progressively thereafter. Pseudotyped rAAV-2cap5 gave peak expression levels on the second day. Consistent with diminishing gene expression levels, the amount of internalized viral DNA following rAAV-2 infection, dropped gradually over the course of three days. However, viral DNA following rAAV-2cap5 infection was both more abundant and more stable despite the low level of gene expression. These findings suggest that difference in viral infection of HeLa cells with rAAV-2 and rAAV-2cap5 is not likely solely due to receptor internalization.

Tripeptyl aldehyde proteosome inhibitors enhance the transduction efficiency of both rAAV-2 and rAAV-2cap5. The 6-fold higher transgene expression of the native rAAV-2 as compared to rAAV-2cap5 virus in HeLa cells does not appear to correlate with increased viral genome internalization. Since the viral genome is identical, viral DNA stability, strand conversion, and the efficiency of gene transcription should also remain the same with both serotypes. Thus, differences in the intracellular processing, facilitated by AAV-2 and AAV-5 capsid entry pathways, might impart alternative fates which effect the efficiency of transduction with these two viruses.

The proteosome system is known to modulate the intracellular processing of many proteins and viruses such as HIV (Schwartz et al., 1998). Previously, that cell-permeable tripeptylaldehyde proteosome inhibitors, such as LLnL or ZLL, were found to substantially augment rAAV-2 mediated gene transfer to the apical surface of polarized cultures of human bronchial epithelial cells and mouse lung in vivo (Duan et al., 2000b). Hence, the proteosome pathway might also affect gene transfer with rAAV-2cap5 virus. To this end, transduction efficiencies of rAAV-2RSVluc and rAAV-2cap5RSVluc were compared in the presence or absence of tripeptyl proteosome inhibitors (40 μM LLnL or 4 μM ZLL). Results from these experiments in four different cell lines are summarized in Table 1. All four cell types tested demonstrated augmentation of both rAAV-2 or rAAV-2cap5 transduction in the presence of LLnL or ZLL. No significant differences in the effect of these inhibitors on the transduction of native and pseudotyped viruses were found for fetal fibroblasts and 293 cells. However, a significantly higher induction of transgene expression was seen following native rAAV-2 infection of HeLa and IB3 cells as compared to that achieved with rAAV-2cap5 virus. These findings suggest that both serotypes of AAV may be susceptible to proteasome barriers.

TABLE 1
Fold induction of luciferase transgene expression
with proteosome inhibitors*

| | 40 μM LLnL | | 4 μM ZLL | |
|---|---|---|---|---|
| | rAAV-2 | rAAV-2cap5 | rAAV-2 | rAAV-2cap5 |
| HeLa | 16 +/− 0.80 | 6.23 +/− 0.32 | 20.34 +/− 4.32 | 4.82 +/− 1.03 |
| Fetal | 30.09 +/− 2.91 | 24.70 +/− 3.33 | 12.05 +/− 1.07 | 10.48 +/− 0.85 |
| Fibroblasts | | | | |
| 293 | 10.38 +/− 2.92 | 7.20 +/− 1.40 | 5.43 +/− 1.29 | 6.25 +/− 0.25 |
| IB3 | 104.94 +/− 0.87 | 24.07 +/− 0.25 | 63.19 +/− 1.23 | 24.58+/− 0.18 |

*Results demonstrate the mean (+/− SEM, N = 4) fold induction of luciferase activity in the presence of proteasome inhibitor.

The effect of different viral MOIs and doses of both inhibitors were also evaluated in HeLa cells (FIG. 12). Cells were pre-incubated with increasing doses of LLnL (up to 100 μM) or ZLL (up to 10 μM) for 1 hour prior to infection with the native rAAV-2 or rAAV-2cap5 (each at 250 particles/cell). The highest doses of the inhibitors were toxic to cells and led to more than 20% cell attrition at 24 hours after infection and hence data is not presented for these conditions. However, concentrations as high as 40 μM LLnL or 4 μM ZLL showed no obvious toxicity to the cells. HeLa cells demonstrated a dose-dependent increase in transduction following LLnL treatment for both serotypes of virus. Subtler differences in the maximal effect of LLnL were seen between the two viruses with peak induction at 40 μM for rAAV-2 and 8 μM for rAAV-2cap5. However, the maximal effect of ZLL was similar for both serotypes and peaked at 0.8 μM (FIG. 12A). The level induction facilitated by 40 μM LLnL was independent of the MOI of infection for both rAAV-2 and rAAV-2cap5 (FIG. 12B). In these experiments, rAAV-2 transduction was approximately 3-fold higher than that seen following infection with pseudotyped rAAV-2cap5 (FIG. 12B).

Both AAV-2 and AAV-5 capsids proteins are substrates for ubiquitination. Proteosome-dependent degradation of ubiquitinated molecules represents a major pathway for disposal of both endogenous and foreign proteins (Pickart, 2001 and Schwartz et al., 1999). Recent studies have also demonstrated that the ubiquitin-proteosome system can regulate receptor-mediated endocytosis (Strous et al., 1999). Previously, AAV-2 capsid proteins were found to be ubiquitinated in human fibroblasts and that LLnL treatment augments rAAV transgene expression 10-fold in this cell type (Duan et al., 2000b).

To test whether AAV-5 capsids are ubiquitinated following infection, immunoprecipitation experiments were performed with anti-capsid antibody followed by western blots with anti-ubiquitin antibody. The B1 antibody recognizes both AAV-5 and AAV-2 capsid proteins with equivalent sensitivity (data not shown). These experiments were performed in fetal fibroblasts, IB3, 293, and HeLa cells. However, due to low level of infection of all cell types but HeLa cells with rAAV-2cap5, insufficient viral recovery prevented conclusive analysis in fetal fibroblasts, IB3, and 293 cells. For example, internalized viral genomes (as determined by Hirt DNA Southern blots) were significantly lower following rAAV-2cap5 infection as compared to rAAV-2 for all cell lines but HeLa cells (data not shown). Since viral uptake in HeLa cells was similar for both rAAV-2 and rAAV-2cap5 virus, comparative analyses in capsid ubiquitination were conducted in this cell line (FIG. 11C). Furthermore, since LLnL similar augmented both rAAV-2 and rAAV-2cap5 transduction (only 2.6-fold divergent), if AAV capsid ubiquitination was linked to responsiveness by proteasome inhibitor, it would be evident in both AAV-2 and AAV-5 capsids.

Results from immunoprecipitation experiments demonstrated that capsid proteins from both rAAV-2 and rAAV-2cap5 virus were ubiquitinated in Hela cells in the presence of LLnL (FIG. 13A, lanes 2 and 6). The presence of proteasome inhibitor was required to see an accumulation of ubiquitinated capsid, as might be expected in these molecules are quickly degraded by the proteasome. Interestingly, if indeed ubiquitinated capsids are targeted to the proteasome for degradation, one would expect that treatment with proteasome inhibitors might also increase the stability of viral genome stability in cells. However, as shown in FIG. 13B, this was not the case for either rAAV-2 or rAAV-2cap5 virus. No change in the abundance of intracellular viral DNA was detected at 24 hours following infection, with or without the presence of LLnL. This result is consistent with a previous report that the presence of LLnL did not substantially prevent enzymatic degradation of internalized AAV-2 viral DNA from the apical side of human airway epithelia cells, despite a significantly increased level of transduction (Duan et al., 2000b). The action of the proteosome inhibitor LLnL has been typically attributed to its selective and reversible inhibition of the proteosome system. However, the augmentation effect of proteosome inhibitors on rAAV-2 or AAV-5 vector transduction may be produced by altering endosomal processing or nuclear trafficking of virus, rather than by simply preventing degradation.

Conjugation of the ubiquitin side chain to the viral capsid proteins resulted in a significant molecular weight gain, which led to alterations in the migration patterns on SDS-PAGE. The high molecular weight smears seen for the AAV-2 or AAV-5 capsid proteins after ubiquitination were consistent with previous results with AAV-2 infected human fibroblasts (Duan et al., 2000b). However, in the current experiments, the high molecular weight smear of ubiquitinated AAV protein in HeLa cells had a lower and more heterogeneous molecular mass than found in the previous study. This could be related to a cell type specific difference. It also appears that the intensity of the high molecular smear from the native rAAV-2 infected cells was more dense than that of the rAAV-2cap5 pseudotyped virus. Although this difference is small, it is interesting that rAAV-2 transduction was 3-fold more responsive to proteasome inhibitors and intracellular rAAV-2 viral genomes were less stable than those derived from rAAV-2cap5 (FIGS. 11C and 12A). Given the fact that equivalent levels of viral DNA are taken up by HeLa cells following infection with rAAV-2 and rAAV-2cap5, the abundance of capsid target molecules is assumed to be similar for both serotypes.

To further substantiate finding of AAV-2 and AAV-5 capsid ubiquitination, in vitro reconstitution experiments were performed to directly determine whether purified intact virions are substrates for ubiquitination. Purified active components of the ubiquitin conjugation system isolated from HeLa cell extracts (Fraction I and Fraction II) were used with purified rAAV-2 or rAAV-2cap5 virus as substrates. Western blot analysis with B1 anti-capsid antibody was used to visualize a migratory increase in capsid caused by the addition of ubiquitin (7.6 kDa). As seen in FIG. 13C (lanes 5-7), rAAV-2cap5 virus was a preferential substrate for ubiquitination in the presence of Fraction II alone, giving rise to a larger molecular weight smear of anti-capsid immunoreactive bands. No appreciable increase in larger molecular weight capsid molecules was detected with rAAV-2 in the presence Fraction II alone (FIG. 13C, lanes 2-4). Interestingly, the addition of Fraction I and II to the conjugation reaction increased the intensity of apparent ubiquitination to both AAV-2 and AAV-5 capsids (lanes 9-14) which was most readily apparent for rAAV-2cap5 virus and significantly less ubiquitination was seen for rAAV-2 under all conditions. Pre-treatment of virus by heating in a boiling water bath resulted in denatured capsids that were ubiquitinated and increased conjugation efficiency (FIG. 14). The intensity of VP-1 and VP-2 were notably less intense following incubation of virus in conjugation buffer in the absence of Fraction I and II for unknown reasons. However, based on comparisons to purified virus in the absence of conjugation extracts Fraction I and II, it appears that VP-3 is the predominant target for ubiquitination under the conditions studied.

Discussion

The significant dissimilarity of the viral genomes, Rep and Cap proteins and ITRs of AAV-2 and AAV-5, forecasts differences in tissue tropism, cellular receptors, host range and possibly even replication mechanisms. Hence, one must consider a multiplicity of potential factors when looking for ways to increase the efficacy of gene transfer with these two types of AAV. Furthermore, differences in reported efficiency of recombinant AAV-2 and AAV-5 for gene transfer also provides an opportunity to learn about biology responsible for the unique functional aspects of these two viruses as vectors. Such differences in biology could provide the foundation for improving vector delivery with many serotypes of AAV. Possible differences in biology include cell membrane receptor binding and endocytosis, intracellular trafficking, uncoating, initiation of secondary strand synthesis and conversion of the ssDNA to its active expressible form, the stability and long-term persistence of the viral genome, and more. However, in the present study, such differences were minimized. For example, in these experiments a rAAV-2 genome was pseudotyped with the AAV-5 capsid to minimize potential differences in viral genomes that might otherwise effect comparisons of gene expression with native rAAV-2 vectors. Furthermore, although a number of cell lines were screened for responsiveness of rAAV infection to proteasome inhibitors, the majority of mechanistic studies were performed on HeLa cells which demonstrate equivalent levels of viral uptake despite for rAAV-2 and rAAV-2cap5 despite their divergent receptor entry pathway (Zabner et al., 2000). This consideration significantly simplified comparative aspects of transduction between rAAV-2 and rAAV-2cap5 virus.

In HeLa cells, rAAV-2 demonstrated a transduction efficiency six times higher than that for pseudotyped rAAV-2cap5 virus. These results support findings by Chlorini et al. (1999) comparing β-galactosidase expression in HeLa cells using native rAAV-2 and rAAV-5 vectors which demonstrated a seven fold higher level of transduction with rAAV-2. Interestingly, the studies described herein demonstrated using Southern blot analysis of low molecular weight Hirt DNA that levels of viral genomes taken up by cells within a 24 hour period were virtually identical for rAAV-2 and rAAV-2cap5. This demonstrates that the differences in AAV-2 and AAV-5 binding and internalization in HeLa cells may be minimal, even through they enter cells though different receptor-mediated mechanisms. Additional, viral DNA introduced into HeLa cells by pseudotyped virus tended to be more resistant to degradation. Together, these results suggest the potential for different endosomal processing and/or nuclear trafficking mechanisms for the two AAV vector serotypes.

A concrete understanding of endocytic and nuclear trafficking mechanisms associated with AAV transduction has remained elusive. Various signaling pathways might play a role in these processes. Previously, it was reported that the ubiquitin-proteosome pathway is involved in AAV-2 transduction. By inhibiting proteosome function, a substantial augmentation in rAAV-2 mediated transgene expression was observed. In the present study, it was demonstrated that proteosome inhibitors enhance not only rAAV-2, but also rAAV-2cap5 mediated gene transfer. The extent of this enhancement was significantly influences by the cell type analyzed. In HeLa and IB3 cells, a higher augmentation effects on AAV-2 transduction were observed relative with AAV-5. This also implied differences in the internalized virus processing between AAV-2 and AAV-5. Given the fact that proteasome inhibitors did not affect viral genome stability, it appears that these inhibitors do not augment transduction by decreasing the degradation of internalized virions.

The function of ubiquitin conjugation of the virus in cellular processing is currently undefined. From the present studies it is clear that both AAV-2 and AAV-5 capsids are ubiquitinated in HeLa cells. Furthermore, co-administration of the viruses with proteosome inhibitor in Hela cells exhibited a correlation of increased transgene expression with the amount of ubiquitinated AAV capsid protein. It is currently difficult to distinguish a causal relationship between viral ubiquitination and enhanced gene transfer in response to proteasome inhibitors.

Several possibilities may explain the functional involvement of ubiquitin/proteasome pathways in both rAAV-2 and rAAV-5 transduction. First, ubiquitination of capsid may be a signal for intracellular rerouting of virus to a “dead-end” endosomal compartment in the absence of complete protease digestion of the relatively resilient capsid. This hypothesis would invoke ubiquitination as a mechanisms of intracellular innate immunity to incoming virus as has been suggested for HIV (Schwartz et al., 1998). In this case, ubiquitination of viral capsids would be detrimental to rAAV's capacity to complete its latent life cycle. A second alternative hypothesis is that ubiquitination of AAV capsid proteins serve as a signal for viral processing such as endosomes escape, nuclear importing, or virus particle disassembly. Since treatment of cells with proteasome inhibitors augment the level of capsid ubiquitination, this alternative explanation might suggest that increased ubiquitination is a positive signal which benefits completion of the rAAV latent life cycle. Despite the lack of a clear mechanism for enhanced transduction in the presence of proteasome inhibitors, these studies suggest that ubiquitination of AAV capsids may be a common component of cellular interaction for both AAV-2 and AAV-5. Since AAV-2 and AAV-5 are the most divergent serotypes of AAV, these mechanisms may also likely apply to other serotypes as well. Further elucidating mechanisms of AAV ubiquitination may have significant therapeutic benefits in the applications of multiple rAAV serotypes for gene therapy.

### EXAMPLE 3

### Proteasome Involvement in rAAV-2 and rAAV-5 Transduction of Polarized Airway Epithelia In Vitro and In Vivo

Inhibition of the proteasome with small tripeptide inhibitors such as LLnL can significantly augment rAAV-2 transduction from the apical membrane of both polarized human airway epithelia in vitro and mouse lung in vivo (Duan et al., 2000). As AAV-5 has been reported to have higher tropism for, and alternate receptors on, the apical membrane of airway epithelia, increased transduction of airway epithelia from the apical membrane with rAAV-5 might be due to altered proteasome involvement. Co-administration of a proteasome inhibitor was found to augment transduction of both serotypes in a cell type dependent manner (see Table 1).

To better understand serotype-specific differences in airway transduction, the effect of proteasome inhibitors on rAAV-2 and rAAV-5 transduction in polarized human airway epithelial cultures and mouse lung was examined. A rAAV-2 proviral construct was packaged into both AAV-2 and AAV-5 capsid to generate AV2.RSVluc and AV2.RSVlucCap5 viruses which express the luciferase transgene. rAAV-2, but not rAVV-5, demonstrated a significant difference in transduction from the apical versus basolateral surface. Transduction with AV2.RSVluc was 36- and 103-fold greater from the basolateral membrane at 5 and 14 days post-infection, respectively. In contrast, AV2.RSVlucCap5 transduced epIthelia from the apical and basolateral membranes with similar efficiencies at both time points.

LLnL augments AV2.RSVluc transduction from the apical and basolateral surfaces. However, application of LLnL selectively increased AV2.RSVlucCap5 transduction 12-fold only when virus was applied to the apical surface. These results suggest an interesting difference in the involvement of the proteasome for various AAV capsid entry pathways that are effected by cell polarity.

The proteasome inhibitor Z-LLL was found to induce long-term (5 month) transduction with rAAV-2 in mouse lung. To determine in vivo transduction efficiency of AV2.RSVlucCap5, mice were infected with 6×10¹⁰ particles of AV2.RSVlucCap5 by nasal aspiration alone (control) or in combination with 200 μM Z-LLL (12 mice per group). Co-administration of Z-LLL induced whole lung luciferase expression 17.2- and 2.1-fold at 14 (2 weeks) and 42 (6 weeks) days post-infection, respectively (FIG. 15). Interestingly, luciferase expression was further reduced at 3 months post-infection (FIG. 16).

These observations suggest a striking difference in the kinetics and longevity of induction by Z-LLL between in vivo studies with rAAV-2 and rAAV-5. Since in vivo transduction is significantly more efficient with rAAV-5 compared to rAAV-2, altering proteasome activity may simply enhance the rate of transduction with rAAV-5. In the case of rAAV-2, this basal rate may be significantly reduced from the apical membrane in vivo rendering more sustained augmentation of transduction by proteasome inhibitors.

### REFERENCES

Bantel-Schaal et al., J. Virol., 73:939-47 (1999).

Bulfield et al., Proc. Natl. Acad. Sci. U.S.A., 81:1189-92 (1984).

Chao et al., Mol. Ther., 2:619-23 (2000).

Chlorini et al., J. Virol., 71:6823-33 (1997).

Chlorini et al., J. Virol., 73:1309-19 (1999).

Chlorini et al., J. Virol., 73:4293-8 (1999).

Cordier et al., Mol. Ther., 1:119-129 (2000).

Cordier et al., Hum. Gene Ther., 12:205-215 (2001).

Davidson et al., Proc. Natl. Acad. Sci. U.S.A., 97:3428-32 (2000).

Duan et al., Virus Res., 48:41-56 (1997).

Duan et al. J. Virol., 72:8568-8577 (1998).

Duan et al., Hum. Gene Ther., 10:1553-1557 (1999).

Duan et al., J. Virol., 73:10371-6 (1999).

Duan et al., J. Clin. Invest., 105:1573-1587 (2000b).

Duan et al., Nat. Med., 6:595-8 (2000a).

Flotte et al., Gene Ther., 2:357-62 (1995).

Ganoth et al., J. Biol. Chem, 263:12412-9 (1988).

Girod et al., Nat. Med., 5:1052-6 (1999).

Greelish et al., Nat. Med., 5:439-43 (1999).

Hagstrom et al., Blood. 95:2536-42 (2000).

Hansen et al., J. Virol., 74:992-6 (2000).

Hansen et al., J. Virol., 75:4080-90 (2001).

Hershko et al., J. Biol. Chem., 258:8206-14 (1983).

Hershko et al., J. Biol. Chem., 261:11992-9 (1986).

Hildinger et al., J. Virol., 75:6199-203 (2001).

Kay et al., Nat. Genet., 24:257-61 (2000).

Li et al., J. Virol., 71:5236-43 (1997).

Li et al., Gene Ther., 6:74-82 (1999).

Melandri et al., Biochemistry, 35:12893-900 (1996).

Muramatsu et al., Virology, 221:208-17 (1996).

Muzyczka, Curr. Top. Microbiol. Immunol., 158:97-129 (1992).

Nakai et al., Nat. Biotechnol., 18:527-32 (2000).

Pickart, Annu. Rev. Biochem., 70:503-533 (2001).

Pickles et al., J. Virol., 74:6050-7 (2000).

Pruchnic et al., Hum. Gene Ther., 11:521-36 (2000).

Qing et al., J. Virol., 72:1593-9 (1998).

Rutledge et al., J. Virol., 72:309-19 (1998).

Samulski et al., J. Virol., 61:3096-101 (1987).

Samulski et al., J. Virol., 63:3822-8 (1989).

Sanlioglu et al., Mol. Therapy, 3:S185, (2001).

Schwartz et al., J. Virol., 72:3845-50 (1998).

Schwartz et al., Annu. Rev. Med., 50:57-74 (1999).

Strous et al., J. Cell Sci., 112:1417-23 (1999).

Summerford et al., J. Virol., 72:1438-45 (1998).

Sun et al., Nat. Med., 6:599-602 (2000).

Thompson et al., In Vitro Cell Dev Biol., 29A:165-70 (1993).

Tripathy et al., Nat Med., 2:545-50 (1996).

Walsh et al., Curr. Opin. Genet. Dev., 7:597-602 (1997).

Walters et al. J. Virol., 74:535-40 (2000).

Walters et al., J. Biol. Chem., 21:21 (2001).

Wang et al., Proc. Natl. Acad. Sci. U.S.A., 97:13714-9 (2000).

Ward et al., Biochemistry, 21:4535-40 (1982).

Wu et al., J. Virol., 74:8635-47 (2000).

Xiao et al., J. Virol., 72:2224-32 1998).

Xiao et al., J. Virol., 73:3994-4003 (1999).

Yaffe et al., Differentiation, 7:159-66 (1977).

Yan et al., Proc. Natl. Acad. Sci. U.S.A., 97:6716-6721 (2000).

Zabner et al., J. Virol., 74:3852-8 (2000).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A method to alter rAAV transduction of a mammalian cell, comprising:
contacting the mammalian cell with at least one rAAV comprising AAV capsid protein and a first recombinant DNA molecule comprising linked:
i) a first DNA segment comprising a 5′-ITR of AAV;
ii) a second DNA segment which does not comprise AAV sequences; and
iii) a third DNA segment comprising a 3′-ITR of AAV, wherein at least one of the ITRs in the first recombinant DNA molecule is from a serotype of AAV that is different than the serotype of AAV for the AAV capsid protein,
and an agent in an amount effective to alter virus transduction.

2. A method to alter rAAV transduction of a mammalian cell, comprising:
contacting the mammalian cell with at least one rAAV comprising AAV-5 capsid protein and a first recombinant DNA molecule comprising linked:
i) a first DNA segment comprising a 5′-ITR of AAV;
ii) a second DNA segment which does not comprise AAV sequences; and
iii) a third DNA segment comprising a 3′-ITR of AAV,
and an agent in an amount effective to alter virus transduction.

3. The method of claim 1 or 2 further comprising contacting the cell with a further rAAV comprising AAV capsid protein and a second recombinant DNA molecule comprising linked:
i) a first DNA segment comprising a 5′-ITR of AAV;
ii) a second DNA segment which does not comprise AAV sequences but which comprises sequences that are different than the sequences in the second DNA segment of the first recombinant DNA molecule; and
iii) a third DNA segment comprising a 3′-ITR of AAV.

4. The method of claim 3 wherein the further rAAV is a pseudotyped rAAV.

5. The method of claim 3 wherein the second DNA segment of the first recombinant DNA molecule comprises a portion of an open reading frame operably linked to a promoter.

6. The method of claim 5 wherein the first recombinant DNA molecule comprises a splice donor site 3′ to the portion of the open reading frame.

7. The method of claim 6 wherein the second DNA segment of the second recombinant DNA molecule comprises a splice acceptor site 5′ to another portion of an open reading frame, which together with the second DNA segment of the first recombinant DNA molecule encodes a functional peptide or polypeptide.

8. The method of claim 3 wherein the second DNA segment of the second recombinant DNA molecule comprises a portion of an open reading frame operably linked to a promoter.

9. The method of claim 8 wherein the second recombinant DNA molecule comprises a splice donor site 3′ to the portion of the open reading frame.

10. The method of claim 9 wherein the second DNA segment of the first recombinant DNA molecule comprises a splice acceptor site 5′ to another portion of an open reading frame, which together with the second DNA segment of the second recombinant DNA molecule encodes a functional peptide or polypeptide.

11. The method of claim 3 wherein the second DNA segment of the first recombinant DNA molecule comprises an enhancer and the second DNA segment of the second recombinant DNA molecule comprises an open reading frame.

12. The method of claim 3 wherein the second DNA segment of the first recombinant DNA molecule comprises a promoter and the second DNA segment of the second recombinant DNA molecule comprises an open reading frame.

13. The method of claim 3 wherein the second DNA segment of the second recombinant DNA molecule comprises an enhancer and the second DNA segment of the first recombinant DNA molecule comprises an open reading frame.

14. The method of claim 3 wherein the second DNA segment of the second recombinant DNA molecule comprises a promoter and the second DNA segment of the first recombinant DNA molecule comprises an open reading frame.

15. The method of claim 3 wherein at least one of the rAAVs has a chimeric ITR or a chimeric genome.

16. The method of claim 1 or 2 wherein the rAAV has a chimeric ITR or a chimeric genome.

17. The method of claim 1 or 2 wherein the cell is a lung cell, an epithelial cell, a muscle cell, a liver cell, or a neuronal cell.

18. The method of claim 7 wherein the cell expresses the functional peptide or polypeptide.

19. The method of claim 18 wherein the functional peptide or polypeptide is a therapeutic peptide or polypeptide.

20. The method of claim 19 wherein the functional polypeptide is cystic fibrosis transmembrane conductance receptor, β-globin, γ-globin, tyrosine hydroxylase, glucocerebrosidase, aryl sulfatase A, factor VIII, dystrophin or erythropoietin.

21. The method of claim 10 wherein the cell expresses the functional peptide or polypeptide.

22. The method of claim 21 wherein the functional peptide or polypeptide is a therapeutic peptide or polypeptide.

23. The method of claim 22 wherein the functional polypeptide is cystic fibrosis transmembrane conductance receptor, β-globin, γ-globin, tyrosine hydroxylase, glucocerebrosidase, aryl sulfatase A, factor VIII, dystrophin or erythropoietin.

24. The method of claim 11 wherein the open reading frame encodes a functional peptide or polypeptide.

25. The method of claim 24 wherein the functional peptide or polypeptide is a therapeutic peptide or polypeptide.

26. The method of claim 25 wherein the functional polypeptide is cystic fibrosis transmembrane conductance receptor, β-globin, γ-globin, tyrosine hydroxylase, glucocerebrosidase, aryl sulfatase A, factor VIII, dystrophin or erythropoietin.

27. The method of claim 12 wherein the open reading frame encodes a functional peptide or polypeptide

28. The method of claim 27 wherein the functional peptide or polypeptide is a therapeutic peptide or polypeptide

29. The method of claim 28 wherein the functional polypeptide is cystic fibrosis transmembrane conductance receptor, β-globin, γ-globin, tyrosine hydroxylase, glucocerebrosidase, aryl sulfatase A, factor VIII, dystrophin or erythropoietin.

30. The method of claim 13 wherein the open reading frame encodes a functional peptide or polypeptide.

31. The method of claim 30 wherein the functional peptide or polypeptide is a therapeutic peptide or polypeptide.

32. The method of claim 31 wherein the functional polypeptide is cystic fibrosis transmembrane conductance receptor, β-globin, γ-globin, tyrosine hydroxylase, glucocerebrosidase, aryl sulfatase A, factor VIII, dystrophin or erythropoietin.

33. The method of claim 14 wherein the open reading frame encodes a functional peptide or polypeptide.

34. The method of claim 33 wherein the functional peptide or polypeptide is a therapeutic peptide or polypeptide.

35. The method of claim 34 wherein the functional polypeptide is cystic fibrosis transmembrane conductance receptor, β-globin, γ-globin, tyrosine hydroxylase, glucocerebrosidase, aryl sulfatase A, factor VIII, dystrophin or erythropoietin.

36. The method of claim 1 or 2 wherein the agent enhances viral transduction.

37. The method of claim 1 or 2 wherein the agent is a proteosome inhibitor.

38. The method of claim 1 or 2 wherein the agent is LLnL or Z-LLL.

39. The method of claim 1 or 2 wherein the agent inhibits the activation of ubiquitin, the transfer of ubiquitin to the ubiquitin carrier protein, ubiquitin ligase, or a combination thereof.

40. The method of claim 1 or 2 wherein the agent inhibits ubiquitin ligase.

41. The method of claim 1 or 2 wherein the agent is H-Leu-Ala-OH, H-His-Ala-OH, or a combination thereof.

42. The method of claim 1 or 2 further comprising administering a second agent that enhances the activity of the agent that alters transduction.

43. The method of claim 42 wherein the second agent is EGTA.

44. A method to express a functional peptide or polypeptide in a host cell, comprising: contacting the host cell with an agent that alters pseudotyped rAAV transduction and at least two rAAVs in an amount effective to express the functional peptide or polypeptide, wherein at least one rAAV is a pseudotyped rAAV, wherein one rAAV comprises AAV capsid protein and a first recombinant DNA molecule comprising linked:
i) a first DNA segment comprising a 5′-ITR of AAV;
ii) a second DNA segment which does not comprise AAV sequences, wherein the second DNA segment comprises an enhancer, a promoter, or at least a portion of an open reading frame which encodes at least a portion of the peptide or polypeptide, or a combination thereof; and
iii) a third DNA segment comprising a 3′-ITR of AAV, wherein at least one of the ITRs in the first recombinant DNA molecule is from a serotype of AAV that is different than the serotype of AAV for the AAV capsid protein;
wherein a second rAAV comprises AAV capsid protein and a second recombinant DNA molecule comprising linked:
i) a first DNA segment comprising a 5′-ITR of AAV;
ii) a second DNA segment which does not comprise AAV sequences but which sequences are different than the sequences in the second DNA segment of the first recombinant DNA molecule, wherein the second DNA segment of the second recombinant DNA molecule encodes the functional peptide or polypeptide if the second DNA segment of the first recombinant DNA molecule does not comprise a portion of the open reading frame and wherein if the second DNA segment of the first recombinant DNA molecule encodes a portion of the open reading, the second DNA segment of the second recombinant DNA molecule comprises a portion of the open reading frame which together with the second DNA segment of the first recombinant DNA molecule encodes the functional peptide or polypeptide; and
iii) a third DNA segment comprising a 3′-ITR of AAV.

45. A method to express a functional peptide or polypeptide in a host cell, comprising: contacting the host cell with an agent that alters pseudotyped rAAV transduction and at least two rAAVs in an amount effective express the functional peptide or polypeptide, wherein at least one rAAV is a pseudotyped rAAV, wherein one rAAV comprises AAV capsid protein and a first recombinant DNA molecule comprising linked:
i) a first DNA segment comprising a 5′-ITR of AAV;
ii) a second DNA segment which does not comprise AAV sequences, wherein the second DNA segmen comprises an enhancer, a promoter, or at least a portion of an open reading frame which encodes a portion of the peptide or polypeptide, or a combination thereof,
iii) a third DNA segment comprising a 3′-ITR of AAV; and
wherein a second rAAV comprises AAV capsid protein and a second recombinant DNA molecule comprising linked
i) a first DNA segment comprising a 5′-ITR of AAV;
ii) a second DNA segment which does not comprise AAV sequences but which sequences are different than the sequences in the second DNA segment of the first recombinant DNA molecule, wherein the second DNA segment of the second recombinant DNA molecule encodes the functional peptide or polypeptide if the second DNA segment of the first recombinant DNA molecule does not comprise a portion of the open reading frame and wherein if the second DNA segment of the first recombinant DNA molecule encodes a portion of the open reading, the second DNA segment of the second recombinant DNA molecule comprises a portion of the open reading frame which together with the second DNA segment of the first recombinant DNA molecule encodes the functional peptide or polypeptide; and
iii) a third DNA segment comprising a 3′-ITR of AAV, wherein at least one of the ITRs in the second recombinant DNA molecule is from a serotype of AAV that is different than the serotype of AAV for the AAV capsid protein.

46. The method of claim 44 or 45 wherein the second DNA segment of the first recombinant DNA molecule comprises a portion of an open reading frame operably linked to a promoter.

47. The method of claim 46 wherein the first recombinant DNA molecule comprises a splice donor site 3′ to the portion of the open reading frame.

48. The method of claim 47 wherein the second DNA segment of the second recombinant DNA molecule comprises a splice acceptor site 5′ to another portion of an open reading frame, which together with the second DNA segment of the first recombinant DNA molecule encodes a functional peptide or polypeptide.

49. The method of claim 44 or 45 wherein the second DNA segment of the first recombinant DNA molecule comprises an enhancer and the second DNA segment of the second recombinant DNA molecule comprises an open reading frame.

50. The method of claim 44 or 45 wherein the second DNA segment of the first recombinant DNA molecule comprises a promoter and the second DNA segment of the second recombinant DNA molecule comprises an open reading frame.

51. The method of claim 44 or 45 wherein at least one of the rAAVs has a chimeric ITR.

52. The method of claim 44 or 45 wherein at least one of the rAAVs has a chimeric genome.

53. The method of claim 44 or 45 wherein the cell is a lung cell, an epithelial cell, a muscle cell, a liver cell, or a neuronal cell.

54. The method of claim 44 or 45 wherein the functional peptide or polypeptide is a therapeutic peptide or polypeptide.

55. The method of claim 60 wherein the functional polypeptide is cystic fibrosis transmembrane receptor, β-globin, γ-globin, tyrosine hydroxylase, glucocerebrosidase, aryl sulfatase A, factor VIII, dystrophin or erythropoietin.

56. The method of claim 44 or 45 wherein the agent is a proteosome inhibitor.

57. The method of claim 44 or 45 wherein the agent is LLnL or Z-LLL.

58. The method of claim 44 or 45 wherein the agent inhibits the activation of ubiquitin, the transfer of ubiquitin to the ubiquitin carrier protein, ubiquitin ligase, or a combination thereof.

59. The method of claim 44 or 45 wherein the agent inhibits ubiquitin ligase.

60. The method of claim 44 or 45 wherein the agent is H-Leu-Ala-OH, H-His-Ala-OH, or a combination thereof.

61. The method of claim 44 or 45 further comprising administering a second agent that enhances the activity of the agent that alters transduction.

62. The method of claim 61 wherein the second agent is EGTA.

63. A method to inhibit or treat a condition associated with the absence of, or reduced or aberrant, expression of an endogenous gene product, comprising:
contacting a mammal at risk of or having said condition with an agent that alters pseudotype rAAV transduction and at least one rAAV comprising a transgene encoding at least a portion of a functional gene product for the corresponding endogenous gene product, in an amount effective to inhibit or treat the condition, wherein at least one rAAV is a pseudotyped rAAV, wherein one rAAV comprises AAV capsid protein and a first recombinant DNA molecule comprising linked:
i) a first DNA segment comprising a 5′-ITR of AAV;
ii) a second DNA segment which does not comprise AAV sequences, wherein the second DNA segment comprises an enhancer, a promoter, or at least a portion of an open reading frame which encodes at least a portion of the functional gene product, or a combination thereof; and
iii) a third DNA segment comprising a 3′-ITR of AAV, wherein at least one of the ITRs in the first recombinant DNA molecule is from a serotype of AAV that is different than the serotype of AAV for the AAV capsid protein;
wherein a second rAAV comprises AAV capsid protein and a second recombinant DNA molecule comprising linked:
i) a first DNA segment comprising a 5′-ITR of AAV;
ii) a second DNA segment which does not comprise AAV sequences but which comprises sequences that are different than the sequences in the second DNA segment of the first recombinant DNA molecule, wherein the second DNA segment of the second recombinant DNA molecule encodes the functional gene product if the second DNA segment of the first recombinant DNA molecule does not comprise a portion of the open reading frame and wherein if the second DNA segment of the first recombinant DNA molecule encodes a portion of the open reading, the second DNA segment of the second recombinant DNA molecule comprises a portion of the open reading frame which together with the second DNA segment of the first recombinant DNA molecule encodes the functional gene product; and
iii) a third DNA segment comprising a 3′-ITR of AAV.

64. A method to inhibit or treat a condition associated with the absence of, or reduced or aberrant, expression of an endogenous gene product, comprising:
contacting a mammal at risk of or having said condition with an agent that alters pseudotype rAAV transduction and at least one rAAV comprising a transgene encoding at least a portion of a functional gene product for the corresponding endogenous gene product, in an amount effective to inhibit or treat the condition, wherein at least one rAAV is a pseudotype rAAV, wherein one rAAV comprises AAV capsid protein and a first recombinant DNA molecule comprising linked:
i) a first DNA segment comprising a 5′-ITR of AAV;
ii) a second DNA segment which does not comprise AAV sequences but which comprises an enhancer, a promoter, or at least a portion of an open reading frame encoding a portion of the functional gene product, or a combination thereof;
iii) a third DNA segment comprising a 3′-ITR of AAV; and
wherein a second rAAV comprises AAV capsid protein and a second recombinant DNA molecule comprising linked
i) a first DNA segment comprising a 5′-ITR of AAV;
ii) a second DNA segment which does not comprise AAV sequences but which comprises sequences that are different than the sequences in the second DNA segment of the first recombinant DNA molecule, wherein the second DNA segment of the second recombinant DNA molecule encodes the functional gene product if the second DNA segment of the first recombinant DNA molecule does not comprise a portion of the open reading frame and wherein if the second DNA segment of the first recombinant DNA molecule encodes a portion of the open reading, the second DNA segment of the second recombinant DNA molecule comprises a portion of the open reading frame which together with the second DNA segment of the first recombinant DNA molecule encodes the functional gene product; and
iii) a third DNA segment comprising a 3′-ITR of AAV, wherein at least one of the ITRs in the second recombinant DNA molecule is from a serotype of AAV that is different than the serotype of AAV for the AAV capsid protein.

65. The method of claim 63 or 64 wherein the transgene encodes at least a portion of cystic fibrosis transmembrane conductance receptor, β-globin, γ-globin, tyrosine hydroxylase, glucocerebrosidase, aryl sulfatase A, factor VIII, dystrophin or erythropoietin.

66. A cell contacted with at least one rAAV comprising AAV capsid protein and a first recombinant DNA molecule comprising linked:
i) a first DNA segment comprising a 5′-ITR of AAV;
ii) a second DNA segment which does not comprise AAV sequences; and
iii) a third DNA segment comprising a 3′-ITR of AAV, wherein at least one of the ITRs in the first recombinant DNA molecule is from a serotype of AAV that is different than the serotype of AAV for the AAV capsid protein,
and an agent in an amount effective to alter virus transduction.

67. A cell contacted with at least one rAAV comprising AAV-5 capsid protein and a first recombinant DNA molecule comprising linked:
i) a first DNA segment comprising a 5′-ITR of AAV;
ii) a second DNA segment which does not comprise AAV sequences; and
iii) a third DNA segment comprising a 3′-ITR of AAV,
and an agent in an amount effective to alter virus transduction.
